SEQUENCE LISTING (59 sequences)

SEQ ID NO: 1 - protein, 88 aa, Artificial sequence
  N-terminal fragment
APIVTCRKLDGREKPFKVDVATAQAQARKAGLTTGKSGDPHRYFAGDHIRWGVNNCDKADAILWEYPIYWVGKNAEWAKDVKTSQQKG

SEQ ID NO: 2 - protein, 41 aa, Artificial sequence
  C-terminal fragment
GPTPIRVVYANSRGAVQYCGVMTHSKVDKNNQGKEFFEKCD

SEQ ID NO: 3 - protein, 12 aa, Artificial sequence
  N-terminal fragment
APIVTCRPKLDG

SEQ ID NO: 4 - protein, 118 aa, Artificial sequence
  C-terminal fragment
REKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 5 - protein, 37 aa, Artificial sequence
  N-terminal fragment
APIVTCRPKLDGREKPFKVDVATAQAQARKAGLTTGK

SEQ ID NO: 6 - protein, 93 aa, Artificial sequence
  C-terminal fragment
SGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 7 - protein, 61 aa, Artificial sequence
  N-terminal fragment
APIVTCRPKLDGREKPFKVDVATAQAQARKAGLTTGKSGDPHRYFAGDHIRWGVNNCDKAD

SEQ ID NO: 8 - protein, 69 aa, Artificial sequence
  C-terminal fragment
AILWEYPIYWVGKNAEWAKDVKTSQQKGGPTPIRVVYANSRGAVQYCGVMTHSKVDKNNQGKEFFEKCD

SEQ ID NO: 9 - protein, 73 aa, Artificial sequence
  N-terminal fragment
APIVTCRPKLDGREKPFKVDVATAQAQARKAGLTTGKSGDPHRYFAGDHIRWGVNNCDKADAILWEYPIYWVG

SEQ ID NO: 10 - protein, 57 aa, Artificial sequence
  C-terminal fragment
KNAEWAKDVKTSQQKGGPTPIRVVYANSRGAVQYCGVMTHSKVDKNNQGKEFFEKCD

SEQ ID NO: 11 - protein, 81 aa, Artificial sequence
  N-terminal fragment
APIVTCRPKLDGREKPFKVDVATAQAQARKAGLTTGKSGDPHRYFAGDHIRWGVNNCDKADAILWEYPIYWVGKNAEWAKD

SEQ ID NO: 12 - protein, 49 aa, Artificial sequence
  C-terminal fragment
VKTSQQKGGPTPIRVVYANSRGAVQYCGVMTHSKVDKNNQGKEFFEKCD

SEQ ID NO: 13 - protein, 86 aa, Artificial sequence
  N-terminal fragment
APIVTCRPKLDGREKPFKVDVATAQAQARKAGLTTGKSGDPHRYFAGDHIRWGVNNCDKADAILWEYPIYWVGKNAEWAKDVKTSQ

SEQ ID NO: 14 - protein, 44 aa, Artificial sequence
  C-terminal fragment
QKGGPTPIRVVYANSRGAVQYCGVMTHSKVDKNNQGKEFFEKCD

SEQ ID NO: 15 - protein, 103 aa, Artificial sequence
  N-terminal fragment
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRG

SEQ ID NO: 16 - protein, 27 aa, Artificial sequence
  C-terminal fragment
AVQYCGVMTHSKVDKNNQGKEFFEKCD

SEQ ID NO: 17 - protein, 119 aa, Artificial sequence
  N-terminal fragment
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKN

SEQ ID NO: 18 - protein, 11 aa, Artificial sequence
  C-terminal fragment
NQGKEFFEKCD

SEQ ID NO: 19 - protein, 9 aa, Artificial sequence
  oligonucleotide
GSGSGHHHH

SEQ ID NO: 20 - DNA, 34 bp, Artificial sequence
  polynucleotide sequence
aagccactgtgtcctgaagaaaagcaaagacatc

SEQ ID NO: 21 - protein, 22 aa, Artificial sequence
  peptide
SYSMEHFRWGKPVGGGSSGGGC

SEQ ID NO: 22 - protein, 22 aa, Artificial sequence
  peptide
  misc_feature (4)..(4): Xaa is Nle
  misc_feature (7)..(7): Phe can be D-phenylalanine
SYSXEHFRWGKPVGGGSSGGGC

SEQ ID NO: 23 - protein, 23 aa, Artificial sequence
  peptide
  misc_feature (17)..(17): Phe can be D-phenylalanine
CSGGGSSGGGSYSMEHFRWGKPV

SEQ ID NO: 24 - protein, 23 aa, Artificial sequence
  peptide
  misc_feature (14)..(14): Xaa is Nle
  misc_feature (17)..(17): Phe can be D-phenylalanine
CSGGGSSGGGSYSXEHFRWGKPV

SEQ ID NO: 25 - protein, 90 aa, Artificial sequence
  N-terminal protein fragment
APIVTCRPKLDGREKPFKVDVATAQAQARKAGLTTGKSGDPHRYFAGDHIRWGVNNCDKADAILWEYPIYWVGKNAEWAKDVKTSQQKGC

SEQ ID NO: 26 - protein, 42 aa, Artificial sequence
  C-terminal protein fragment
CGPTPIRVVYANSRGAVQYCGVMTHSKVDKNNQGKEFFEKCD

SEQ ID NO: 27 - protein, 4 aa, Artificial sequence
  Factor Xa protease cleavage site
IEGR

SEQ ID NO: 28 - DNA, 60 bp, Artificial sequence
  bottle haplomer nucleotide sequence
actcgagacgtctccttgtctttgcttttcttcaggacacagtggcgagacgtctcgagt

SEQ ID NO: 29 - DNA, 51 bp, Artificial sequence
  bottle haplomer nucleotide sequence
actcgagacgtctccttcctgcccctcctcctgctccgagacgtctcgagt

SEQ ID NO: 30 - DNA, 11 bp, Artificial sequence
  second haplomer nucleotide sequence
agctctcgagt

SEQ ID NO: 31 - DNA, 12 bp, Artificial sequence
  second haplomer nucleotide sequence
gacgtctcgagt

SEQ ID NO: 32 - DNA, 60 bp, Artificial sequence; identical to SEQ ID NO: 28
  polynucleotide of the bottle haplomer nucleotide sequence

SEQ ID NO: 33 - protein, 157 aa, Artificial sequence
  N-terminal fragment of the superfolder GFP
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQ

SEQ ID NO: 34 - protein, 81 aa, Artificial sequence
  C-terminal fragment of the superfolder GFP
KNGIKANFKIRHNVEDGSVQLADHYQQNTPIGDGPVLLPDNHYLSTQSVLSKDPNEKRDHMVLLEFVTAAGITHGMDELYK

SEQ ID NO: 35 - protein, 157 aa, Artificial sequence
  fragment of superfolder GFP (sfGFP)
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQ

SEQ ID NO: 36 - protein, 230 aa, Artificial sequence
  N-terminal fragment of the Renilla luciferase
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGY

SEQ ID NO: 37 - protein, 83 aa, Artificial sequence
  C-terminal fragment of the Renilla luciferase
KPDVVQIVRNYNAYLRASDDLPKMFIESDPGFFSNAIVEGAKKFPNTEFVKVKGLHFSQEDAPDEMGKYIKSFVERVLKNEQZ

SEQ ID NO: 38 - protein, 229 aa, Artificial sequence
  fragment of Renilla luciferase
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGG

SEQ ID NO: 39 - protein, 82 aa, Artificial sequence
  fragment of Renilla luciferase
KPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 40 - protein, 34 aa, Artificial sequence
  N-terminal fragment
APIVTCRPKLDGREKPFKVDVATAQAQARKAGLT

SEQ ID NO: 41 - protein, 96 aa, Artificial sequence
  C-terminal fragment
TGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 42 - protein, 93 aa, Artificial sequence
  C-terminal fragment of the Gaussia princeps luciferase
MKPTENNEDFNIVAVASNFATTDLDADRGKLPGKKLPLEVLKEMEANARKAGCTRGCLICLSHIKCTPKMKKFIPGRCHTYEGDKESAQGGIG

SEQ ID NO: 43 - protein, 76 aa, Artificial sequence
  C-terminal fragment of the Gaussia princeps luciferase
EAIVDIPEIPGFKDLEPMEQFIAQVDLCVDCTTGCLKGLANVQCSDLLKKWLPQRCATFASKIQGQVDKIKGAGGD

SEQ ID NO: 44 - protein, 5 aa, Artificial sequence
  enterokinase recognition signal
DDDDK

SEQ ID NO: 45 - DNA, 17 bp, Artificial sequence
  oligonucleotide
tttcttcaggacacagc

SEQ ID NO: 46 - DNA, 34 bp, Artificial sequence
  appended template tag
gatgtctttgcttttcttcaggacacagtggctt

SEQ ID NO: 47 - protein, 89 aa, Artificial sequence
  N-terminal
APIVTCRKLDGREKPFKVDVATAQAQARKAGLTTGKSGDPHRYFAGDHIRWGVNNCDKADAILWEYPIYWVGKNAEWAKDVKTSQQKGC

SEQ ID NO: 48 - RNA, 35 nt, Artificial sequence
  synthetic 35-mer corresponding to sarcin-ricin loop in 28S RNA
gguaauccugcucaguacgagaggaaccgcagguu

SEQ ID NO: 49 - DNA, 55 bp, Artificial sequence
  scrambled oligonucleotide
taactgtcaaaagccacaagcggaataatgacttcccagggatagatcaaaaagc

SEQ ID NO: 50 - protein, 130 aa, Artificial sequence
  Hirsutellin A
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 51 - protein, 34 aa, Artificial sequence; identical to SEQ ID NO: 40
  Hirsutellin A dithreonine site N-terminal fragment

SEQ ID NO: 52 - protein, 89 aa, Artificial sequence
  Hirsutellin A diglycine site N-terminal fragment
APIVTCRPKLDGREKPFKVDVATAQAQARKAGLTTGKSGDPHRYFAGDHIRWGVNNCDKADAILWEYPIYWVGKNAEWAKDVKTSQQKG

SEQ ID NO: 53 - protein, 167 aa, Artificial sequence
  sfGFP N-terminal fragment
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQGGSGHHHHHH

SEQ ID NO: 54 - protein, 92 aa, Artificial sequence
  sfGFP C-terminal fragment
MHHHHHHGGSGKNGIKANFKIRHNVEDGSVQLADHYQQNTPIGDGPVLLPDNHYLSTQSVLSKDPNEKRDHMVLLEFVTAAGITHGMDELYK

SEQ ID NO: 55 - protein, 34 aa, Artificial sequence
  S. cerevisiae ubiquitin amino acids 1-34
MQIFVKTLTGKTITLEVESSDTIDNVKSKIQDKE

SEQ ID NO: 56 - protein, 42 aa, Artificial sequence
  S. cerevisiae ubiquitin amino acids 35-76
GIPPDQQRLIFAGKQLEDGRTLSDYNIQKESTLHLVLRLRGG

SEQ ID NO: 57 - protein, 15 aa, Artificial sequence
  serine-glycine linker
SGGGGSGGGGSGGGG

SEQ ID NO: 58 - DNA, 15 bp, Artificial sequence
  HPV-B2-TrisAm
tttgacgtctcgagt

SEQ ID NO: 59 - DNA, 56 bp, Artificial sequence
  Lk-SP-haplomer
taactgtcaaaagccactgtgtcctgaagaaaagcaaagacatctggacaaaaagc

(19) United States
(12) Patent Application Publication
(10) Pub. No.: US 2023/0193244 A1
(43) Pub. Date: Jun. 22, 2023

(54) Methods For Split-Protein Template Assembly By Proximity-Enhanced Reactivity

(71) Applicant: TriBiotica LLC, Madison, WI (US)

(72) Inventors: Ian Dunn, Madison, WI (US); Matthew Lawler, Madison, WI (US)

(21) Appl. No.: 16/462,337

(22) PCT Filed: Nov. 16, 2017
(86) PCT No.: PCT/US2017/061907

§ 371 Date: May 20, 2019

Related U.S. Application Data
(60) Provisional application No. 62/424,689, filed on Nov. 21, 2016.

(51) Int. Cl. C12N 15/10; C12N 15/11

(52) U.S. Cl. CPC C12N 15/1055; C12N 15/1068; C12N 15/1086; C12N 15/11; C12N 2310/531; C12N 2310/3513

(57) ABSTRACT

Compounds, composition, and kits are provided for use in methods for the assisted folding of protein fragments of a of choice point larger protein by means of induced proximity, forced by specific nucleic acid hybridizations between a target nucleic acid molecule and complementary nucleic acid molecules appended to the protein fragments of interest.

### FIELD

The present disclosure is directed, in part, to compounds, composition, and kits for use in methods for the assisted folding of protein fragments of a larger protein by means of induced proximity, forced by specific nucleic acid hybridizations between a target nucleic acid molecule and complementary nucleic acid molecules appended to the protein fragments of interest.

### Background

A goal of drug development is delivering potent bio-therapeutic interventions to pathogenic cells, such as virus infected cells, neoplastic cells, cells producing an autoimmune response, and other dysregulated or dysfunctional cells. Examples of potent bio-therapeutic interventions capable of combating pathogenic cells include toxins, pro-apoptotic agents, and immunotherapy approaches that re-direct immune cells to eliminate pathogenic cells. Unfortunately, developing these agents is extremely difficult because of the high risk of toxicity to adjacent normal cells or the overall health of the patient.

A method that has emerged to allow delivery of potent interventions to pathogenic cells while mitigating toxicity to normal cells is targeting of therapeutics by directing them against molecular markers specific for pathogenic cells. Targeted therapeutics have shown extraordinary clinical results in restricted cases, but are currently limited in their applicability due to a lack of accessible markers for targeted therapy. It is extremely difficult, and often impossible, to discover protein markers for many pathogenic cell types.

More recently, therapies targeted to nucleic acid targets specific to pathogenic cells have been developed. Existing nucleic acid-targeted therapies, such as siRNA, are able to down-modulate expression of potentially dangerous genes, but do not deliver potent cytotoxic or cytostatic interventions and thus are not particularly efficient at eliminating the dangerous cells themselves.

Hence, there exists a need to combat the poor efficacy and/or severe side effects of existing bio-therapeutic interventions. Unlike other forms of protein complementation (such as the alpha-complementation of beta-galactosidase) where pre-folded subunits interact, the methods described herein incolve split-protein approaches characterized by the facilitation of mature folding pathways through enforced spatial proximity. Consequently, split-protein fragments in isolation cannot recapitulate the functional profile of their corresponding parental protein, and fragment background functional levels are accordingly extremely low.

### SUMMARY

Methods are generally described herein whereby split protein refolding can be directed through nucleic acid templates in multiple distinct architectures. In particular, cellular RNAs, or any accessible nucleic acid template sequence within a target cell, can be used for the assembly of specific polypeptide fragments of a protein of interest into functional folded forms. Assembly of, for example, potent ribotoxins in this manner can be used for targeted killing of cells expressing specific markers, including tumor cells or aberrant immune cells.

The present disclosure provides bottle haplomers comprising a polynucleotide that comprises: a) a first 3′ stem portion comprising from about 10 to about 20 nucleotide bases; b) an anti-target loop portion comprising from about 16 to about 40 nucleotide bases linked to the first 3′ stem portion, wherein the anti-target loop portion is substantially complementary to a target nucleic acid molecule; and c) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide comprises a —SH moiety; and wherein the Tₘ of the anti-target loop portion:target nucleic acid molecule is greater than the Tₘ of the first stem portion:second stem portion.

The present disclosure also provides bottle haplomers comprising a polynucleotide that comprises: a) a first 3′ stem portion comprising from about 10 to about 20 nucleotide bases; b) an anti-target loop portion comprising from about 16 to about 40 nucleotide bases linked to the first 3′ stem portion, wherein the anti-target loop portion is substantially complementary to a target nucleic acid molecule; and c) a second 5′ stem portion comprising from about 10 to about 20 nucleotide bases linked to the anti-target loop portion, wherein the first 3′ stem portion is substantially complementary to the second 5′ stem portion; wherein the Tₘ of the anti-target loop portion:target nucleic acid molecule is greater than the Tₘ of the first stem portion:second stem portion; and wherein the 5′ terminus or 3′ terminus of the polynucleotide is linked to the C-terminus of an N-terminal protein fragment or the N-terminus of a C-terminal protein fragment, wherein the terminus of the protein fragment lined to the polynucleotide comprises a cysteine or selenocysteine.

The present disclosure also provides haplomers comprising: a) a polynucleotide; and b) an N-terminal protein fragment or a C-terminal protein fragment, wherein the 3′ or 5′ terminus of the polynucleotide is linked to the N-terminus of the C-terminal protein fragment or the C-terminus of the N-terminal protein fragment; wherein: i) the N-terminal fragment comprises the amino acid sequence of APIVTCRKLDGREKPFKVDVATAQAQARKAGLTTGKSGDP HRYFAGDHIRWGVNNCDKADAILWEYPIYWVGKNAEWAKDVKTSQQKG (SEQ ID NO:1), and the C-terminal fragment comprises the amino acid sequence of GPTPIRVVYANS RGAVQYCGVMTHSKVDKNNQGKEFFEKCD (SEQ ID NO:2); ii) the N-terminal fragment comprises the amino acid sequence of APIVTCRPKLDG (SEQ ID NO:3), and the C-terminal fragment comprises the amino acid sequence of REKPFKVDVATAQAQARKAGLTTGKSGDP HRYFAGDHIRWGVNNCDKADAILWEYPIYWVGKNAEWAKDVKTSQQKGGPTPIRVVY ANSRGAVQYCGVMTHSKVDKNNQGKEFFEKCD (SEQ ID NO:4); iii) the N-terminal fragment comprises the amino acid sequence of APIVTCRPKLDGREKPFKVDVATAQAQAR KAGLTTGK (SEQ ID NO:5), and the C-terminal fragment comprises the amino acid sequence of SGDPHRYFAGDHIRWGVNNCDKADAILWEYPIYWVGKNAEWAKDVKTSQQKGGPT PIRVVYANSRGAVQYCGVMTHSKVDKNNQGKEFFEKCD (SEQ ID NO:6); iv) the N-terminal fragment comprises the amino acid sequence of APIVTCRPKLDGREKPFKVDVA TAQAQARKAGLTTGKSGDPHRYFAGDHIRWGVNNCDKAD (SEQ ID NO:7), and the C-terminal fragment comprises the amino acid sequence of AILWEYPIYWVGKNAEWAKD VKTSQQKGGPTPIRVVYANSRGAVQYCGVMTHSKVDKNNQGKEFFEKCD (SEQ ID NO:8); v) the N-terminal fragment comprises the amino acid sequence of APIVTCRPKLDGRE KPFKVDVATAQAQARKAGLTTGKSGDPHRYFAGDHIRWGVNNCDKADAILWEYPIYW VG (SEQ ID NO:9), and the C-terminal fragment comprises the amino acid sequence of KNAE WAKDVKTSQQKGGPTPIRVVYANSRGAVQYCGVMTHSKVDKNNQGKEFFEKCD (SEQ ID NO:10); vi) the N-terminal fragment comprises the amino acid sequence of APIVTCRPKLD GREKPFKVDVATAQAQARKAGLTTGKSGDPHRYFAGDHIRWGVNNCDKADAILWEYP IYWVGKNAEWAKD (SEQ ID NO: 11), and the C-terminal fragment comprises the amino acid sequence of VKTSQQKGGPTPIRVVYANSRGAVQYCGVMTHSKVDKNNQGKEFFEKCD (SEQ ID NO:12); vii) the N-terminal fragment comprises the amino acid sequence of APIVTCRPKLDGREKPFKVDVATAQAQARKAGLTTGKSGDPHRYFAGDHIRWGVNNC DKADAILWEYPIYWVGKNAEWAKDVKTSQ (SEQ ID NO:13), and the C-terminal fragment comprises the amino acid sequence of QKGGPTPIRVVYANSRGAVQYCGVMTHS KVDKNNQGKEFFEKCD (SEQ ID NO:14); viii) the N-terminal fragment comprises the amino acid sequence of APIVTCRPKLDGREKPFKVDVATAQAQARKAGLTTGKSGDPHRYFAG DHIRWGVNNCDKADAILWEYPIYWVGKNAEWAKDVKTSQQKGGPTPIRVVYANSRG (SEQ ID NO:15), and the C-terminal fragment comprises the amino acid sequence of AVQYCG VMTHSKVDKNNQGKEFFEKCD (SEQ ID NO:16); ix) the N-terminal fragment comprises the amino acid sequence of APIVTCRPKLDGREKPFKVDVATAQAQARKAGLTTGKSGDPHR YFAGDHIRWGVNNCDKADAILWEYPIYWVGKNAEWAKDVKTSQQKGGPTPIRVVYAN SRGAVQYCGVMTHSKVDKN (SEQ ID NO:17), and the C-terminal fragment comprises the amino acid sequence of NQGKEFFEKCD (SEQ ID NO:18); or the N-terminal fragment comprises the amino acid sequence of APIVTCRPKLDGREKPFKVDVATAQAQARKAGLT (SEQ ID NO:40), and the C-terminal fragment comprises the amino acid sequence of TGKSGD PHRYFAGDHIRWGVNNCDKADAILWEYPIYWVGKNAEWAKDVKTSQQKGGPTPIRVV YANSRGAVQYCGVMTHSKVDKNNQGKEFFEKCD (SEQ ID NO:41).

The present disclosure also provides surface target compounds comprising: a) a template polynucleotide; and b) a peptide; wherein the 5′ terminus of the polynucleotide is coupled to the N-terminus or C-terminus of the peptide, or the 3′ terminus of the polynucleotide is coupled to the N-terminus or C-terminus of the peptide; and wherein the peptide is a ligand for a cell-surface molecule.

The present disclosure also provides fusion proteins comprising: a) an N-terminal protein fragment, a fusion partner protein, and a purification domain, wherein the C-terminus of the N-terminal protein fragment is coupled to the N-terminus of the fusion partner protein, and the C-terminus of the fusion partner protein is coupled to the N-terminus of the purification domain; or b) an N-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the N-terminal protein fragment, wherein the N-terminal protein fragment comprises an N-terminal methionine and a C-terminal cysteine; or c) a C-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the C-terminal protein fragment, wherein the C-terminal protein fragment comprises an N-terminal cysteine.

The present disclosure also provides compounds having the formula

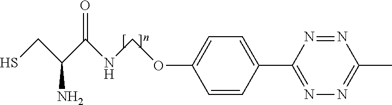

wherein n is from about 3 to about 6.

The present disclosure also provides compositions or kits comprising: a) a first haplomer, wherein the first haplomer comprises a polynucleotide linked to the C-terminus of an N-terminal protein fragment; and b) a second haplomer, wherein the second haplomer comprises a polynucleotide linked to the N-terminus of a C-terminal protein fragment; wherein the polynucleotide of one of the first or second haplomers is linked at its 5′ terminus to the protein fragment, and the other of the first and second haplomers is linked at its 3′ terminus to the protein fragment; wherein the N-terminal protein fragment and the C-terminal protein fragment are derived from a single protein; and wherein: i) the polynucleotide of the first haplomer is complementary to the polynucleotide of the second haplomer; or ii) the polynucleotide of the first haplomer is complementary to a target nucleic acid molecule, and the polynucleotide of the second haplomer is substantially complementary to the target nucleic acid molecule at a site in spatial proximity to the polynucleotide of the first haplomer; or iii) the polynucleotide of the first haplomer is substantially complementary to a portion of a target nucleic acid molecule 5′ adjacent to a stem-loop structure, and the polynucleotide of the second haplomer is substantially complementary to a portion of the target nucleic acid molecule 3′ adjacent to the stem-loop structure; or iv) the polynucleotide of the first haplomer is substantially complementary to a 5′ portion of a loop of a stem-loop structure of a target nucleic acid molecule, and the polynucleotide of the second haplomer is substantially complementary to a 3′ portion of the loop of the stem-loop structure of the target nucleic acid molecule.

The present disclosure also provides compositions or kits comprising: a) a bottle haplomer comprising a polynucleotide that comprises: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide comprises a —SH moiety; wherein the Tₘ of the anti-target loop portion:target nucleic acid molecule is greater than the Tₘ of the first stem portion:second stem portion; b) an N-terminal protein fragment, wherein the C-terminus of the N-terminal protein fragment comprises a cysteine-SH moiety; and c) a bis-maleimide reagent.

The present disclosure also provides compositions or kits comprising: a) a bottle haplomer comprising a polynucleotide that comprises: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and b) a second haplomer comprising a polynucleotide and a C-terminal protein fragment, wherein the 3′ terminus of the polynucleotide is linked to the N-terminus of the C-terminal protein fragment, wherein the N-terminus comprises a cysteine; wherein the polynucleotide of the second haplomer is substantially complementary to the second 5′ stem portion of the polynucleotide of the bottle haplomer; wherein the Tₘ of the anti-target loop portion:target nucleic acid molecule is greater than the Tₘ of the first stem portion:second stem portion; and wherein the N-terminal protein fragment and the C-terminal protein fragment are derived from a single protein.

The present disclosure also provides haplomers, bottle haplomers, fusion proteins, and kits or compositions, as set forth above and herein, wherein the N-terminal protein fragment and C-terminal protein fragment are both derived from a reporter protein, a transcription factor, a signal transduction pathway factor, a gene editing protein, a single-chain immunoglobulin variable region (scFv) protein, a toxic protein, or an enzyme.

The present disclosure also provides methods for the directed assembly of a protein in a cell comprising: a) contacting a cell with a first haplomer comprising a polynucleotide linked to the C-terminus of an N-terminal protein fragment; and b) contacting the cell with a second haplomer comprising a polynucleotide linked to the N-terminus of a C-terminal protein fragment; wherein the polynucleotide of one of the first or second haplomers is linked at its 5′ terminus to the protein fragment, and the other of the first and second haplomers is linked at its 3′ terminus to the protein fragment; wherein the N-terminal protein fragment and the C-terminal protein fragment are derived from a single protein; and wherein: i) the polynucleotide of the first haplomer is substantially complementary to the polynucleotide of the second haplomer; or ii) the polynucleotide of the first haplomer is substantially complementary to a target nucleic acid molecule, and the polynucleotide of the second haplomer is substantially complementary to the target nucleic acid molecule at a site in spatial proximity to the polynucleotide of the first haplomer; or iii) the polynucleotide of the first haplomer is substantially complementary to a portion of a target nucleic acid molecule 5′ adjacent to a stem-loop structure, and the polynucleotide of the second haplomer is substantially complementary to a portion of the target nucleic acid molecule 3′ adjacent to the stem-loop structure; or iv) the polynucleotide of the first haplomer is substantially complementary to a 5′ portion of a loop of a stem-loop structure of a target nucleic acid molecule, and the polynucleotide of the second haplomer is substantially complementary to a 3′ portion of the loop of the stem-loop structure of the target nucleic acid molecule; thereby resulting in the assembly of the protein from the N-terminal protein fragment and the C-terminal protein fragment.

The present disclosure also provides methods for the directed assembly of a protein comprising: a) contacting a target nucleic acid molecule with a bottle haplomer comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; c) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer; wherein the N-terminal protein fragment and the C-terminal protein fragment are derived from a single protein; wherein the Tₘ of the anti-target loop portion:target nucleic acid molecule is greater than the Tₘ of the first stem portion:second stem portion; and wherein the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

The present disclosure also provides methods for the directed assembly of a protein comprising: a) contacting a cell with a surface target compound comprising: i) a template polynucleotide; and ii) a peptide; wherein the 5′ terminus of the polynucleotide is coupled to the N-terminus or C-terminus of the peptide, or the 3′ terminus of the polynucleotide is coupled to the N-terminus or C-terminus of the peptide; wherein the peptide is a ligand for a cell-surface molecule; b) contacting the cell with a first haplomer comprising a polynucleotide linked to the C-terminus of an N-terminal protein fragment; and c) contacting the cell with a second haplomer comprising a polynucleotide linked to the N-terminus of a C-terminal protein fragment; wherein the polynucleotide of one of the first or second haplomers is linked at its 5′ terminus to the protein fragment, and the other of the first and second haplomers is linked at its 3′ terminus to the protein fragment; wherein the N-terminal protein fragment and the C-terminal protein fragment are derived from a single protein; and wherein the polynucleotide of the first haplomer is substantially complementary to the template polynucleotide of the surface target compound, and the polynucleotide of the second haplomer is substantially complementary to the template polynucleotide of the surface target compound at a site in spatial proximity to the polynucleotide of the first haplomer; thereby resulting in the assembly of the protein from the N-terminal protein fragment and the C-terminal protein fragment.

The present disclosure also provides methods for the directed assembly of a protein comprising: a) contacting a cell with a surface target compound comprising: i) a template polynucleotide; and ii) a peptide; wherein the 5′ terminus of the polynucleotide is coupled to the N-terminus or C-terminus of the peptide, or the 3′ terminus of the polynucleotide is coupled to the N-terminus or C-terminus of the peptide; wherein the peptide is a ligand for a cell-surface molecule; b) contacting a target nucleic acid molecule with a bottle haplomer comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to the template polynucleotide of the surface target compound; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; c) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer; wherein the N-terminal protein fragment and the C-terminal protein fragment are derived from a single protein; wherein the Tₘ of the anti-target loop portion:target nucleic acid molecule is greater than the Tₘ of the first stem portion:second stem portion; and wherein the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

The present disclosure also provides methods of cleaving an N-terminal protein fragment from an intein fusion partner in a fusion protein comprising: a) contacting the fusion protein with 2-mercaptoethane sulfonic acid; and b) contacting the fusion protein with a cysteine having a methyltetrazine group; thereby releasing the N-terminal protein fragment from the fusion protein.

### BRIEF DESCRIPTION OF THE DRAWINGS

FIG. 1 shows a schematic for a Protein Complementation Assay (PCA)/Split Protein Technology for protein fragments.

FIG. 2 shows a schematic for a Protein Complementation Assay (PCA)/Split Protein Technology for the expression of protein fragment fusions and co-folding.

FIG. 3 shows a representative schematic for Split-Protein Template Assembly by Proximity-Enhanced Reactivity (SP-TAPER) for a first orientation (panel A) and a second orientation (panel B) of polypeptide-nucleic acid conjugations.

FIG. 4 shows a representative first architecture for SP-TAPER, where appended nucleic acid tags are self-complementary.

FIG. 5 shows a representative second architecture for SP-TAPER, where appended nucleic acid tags are not self-complementary, but hybridize in juxtaposition on a linear target nucleic acid template.

FIG. 6 shows a representative third architecture for SP-TAPER, where the template-mediated polypeptide fragment juxtaposition is directed by a stem-loop structure.

FIG. 7 shows a representative fourth architecture for SP-TAPER, where the template-mediated polypeptide fragment juxtaposition is directed by via an “exo” configuration of hybridization sites within a loop structure.

FIG. 8 shows representative structures of locked TAPER oligonucleotides for SP-TAPER.

FIG. 9 shows a representative schematic of SP-TAPER in concert with the locked TAPER approach.

FIG. 10 shows Hirsutellin A structure and amino acid sequence (SEQ ID NO:50) and representative candidate fragment sequences (e.g., SEQ ID NO:51 and SEQ ID NO:41; and SEQ ID NO:52 and SEQ ID NO:2), showing two representative cleavage sites (e.g., dithreonine SP site and diglycine SP site) for split-protein assays.

FIG. 11 shows representative superfolder GFP (sfGFP) vs. Renilla N-terminal fragments.

FIG. 12 shows a representative sfGFP N-terminal fragment (about 17 kD) for SP-TAPER—intein fusion cleavage.

FIG. 13 shows representative sfGFP and Renilla C-terminal fragments in a maltose-binding protein system, enterokinase cleavage site (SEQ ID NO:44), and enterokinase fragment cleavage.

FIG. 14 shows representative sfGFP and Renilla N-terminal fragment in a maltose-binding protein system, and enterokinase cleavage site (SEQ ID NO:44).

FIG. 15 shows representative enterokinase cleavage of sfGFP N-terminal fragment in a maltose-binding protein system.

FIG. 16 shows an analysis of a representative oligonucleotide (SEQ ID NO:45) with a 5′-disulfide group, after treatment with tris(2-carboxyethyl)phosphine (TCEP), and subsequent reaction with 1,8-bis(maleimido) diethylene glycol (BMP2).

FIG. 17 shows a representative use of a derivative of alpha-melanocyte stimulating hormone (MSH) (SEQ ID NO:21) for the generation of surface template in target cells expressing MC1R with representative templating sequence (SEQ ID NO:20).

FIG. 18 shows representative structures of Hirsutellin A segments (SEQ ID NO:25 and SEQ ID NO:26) and enterokinase cleavage site (SEQ ID NO:44), fragmented at the 89-90 diglycine and expressed as fusion proteins in the MBP system.

FIG. 19 shows representative coupling of oligonucleotides for SP-TAPER by covalently modifying nucleic acid 5′ or 3′ termini with a chelating agent to enable oligonucleotide binding to hexahistidine split-protein fragment fusions.

FIG. 20 shows representative schematic of the removal of excess NTA::Ni oligonucleotides from reactions forming complexes with hexahistidine, by means of a biotinylated oligonucleotide with a tetrahistidine sequence (biotin-GSGSGHHHH; SEQ ID NO:19) by means of solid-phase streptavidin preparations.

FIG. 21 (panels A and B) shows a representative process for preparation of tris-tandem NTA-modified oligonucleotides, and demonstration of product formation on a Locked—TAPER oligonucleotide.

FIG. 22 shows a representative functional strategy for purifying His-binding Tris-tandem NTA Locked-TAPER oligos.

FIG. 23 shows expression of sfGFP fragments (SEQ ID NO:53 and SEQ ID NO:54) as hexahistidine fusions.

FIG. 24 shows SP-TAPER with sfGFP-hexahistidine Locked TAPER oligo-NTA-Ni conjugates.

FIG. 25 shows SP-TAPER with sfGFP fragments.

### DESCRIPTION OF EMBODIMENTS

Certain exemplary embodiments will now be described to provide an overall understanding of the principles of the structure, function, manufacture, and use of the compositions and methods disclosed herein. One or more examples of these embodiments are illustrated in the accompanying drawings. Those skilled in the art will understand that the compositions and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

As used herein, the singular forms “a,” “an,” and “the” include plural references unless the content clearly dictates otherwise. The terms used in this disclosure adhere to standard definitions generally accepted by those having ordinary skill in the art. In case any further explanation might be needed, some terms have been further elucidated below.

As used herein, the phrase “anti-target loop portion” refers to a portion of a bottle haplomer that facilitates sequence-specific binding to a target nucleic acid molecule.

As used herein, the term “base” refers to a molecule containing a purine or pyrimidine group, or an artificial analogue, that forms a binding pair with another corresponding base via Watson-Crick or Hoogsteen bonding interactions. Bases further contain groups that facilitate covalently joining multiple bases together in a polymer, such as an oligomer. Non-limiting examples include nucleotides, nucleosides, peptide nucleic acid residues, or morpholino residues.

As used herein, the terms “bind,” “binds,” “binding,” and “bound” refer to a stable interaction between two molecules that are close to one another. The terms include physical interactions, such as chemical bonds (either directly linked or through intermediate structures), as well as non-physical interactions and attractive forces, such as electrostatic attraction, hydrogen bonding, and van der Waals/dispersion forces.

As used herein, the phrase “bioconjugation chemistry” refers to the chemical synthesis strategies and reagents that ligate common functional groups together under mild conditions, facilitating the modular construction of multi-moiety compounds.

As used herein, the phrase “chemical linker” or “linker” refers to a molecule that binds one haplomer to another haplomer or one moiety to another moiety on different compounds. A linker may be comprised of branched or unbranched covalently bonded molecular chains.

As used herein, the phrase “dosage unit form” refers to physically discrete units suited as unitary dosages for the subjects to be treated.

As used herein, the term “haplomer” refers to nucleic acid molecules linked to a fragment of a protein that bind to a target nucleic acid molecule template in a sequence-specific manner and participate in protein formation during nucleic acid templated assembly. Also included herein are “derivatives” or “analogs” such as salts, hydrates, solvates thereof, or other molecules that have been subjected to chemical modification and maintain the same biological activity or lack of biological activity, and/or ability to act as a haplomer, or function in a manner consistent with a haplomer.

As used herein, the phrase “non-traceless bio-orthogonal chemistry” refers to a reaction involving selectively-reactive moieties in which part or all of the structure of the selectively-reactive moieties is retained in the product structure.

As used herein, the phrase “nucleic acid templated assembly” refers to the production of a protein on a target nucleic acid molecule, such that the protein formation can be facilitated by haplomers being assembled in proximity when bound to a target nucleic acid molecule.

As used herein, the terms “oligomer” and “oligo” refer to a molecule comprised of multiple units where some or all of the units are bases capable of forming Watson-Crick or Hoogsteen base-pairing interactions, allowing sequence-specific binding to nucleic acid molecules in a duplex or multiplex structure. Non-limiting examples include, but are not limited to, oligonucleotides, peptide nucleic acid oligomers, and morpholino oligomers.

As used herein, the phrase “pathogenic cell” can refer to a cell that is capable of causing or promoting a diseased or an abnormal condition, such as a cell infected with a virus, a tumor cell, and a cell infected with a microbe, or a cell that produces a molecule that induces or mediates diseases that include, but are not limited to allergy, anaphylaxis, inflammation and autoimmunity.

As used herein, the phrase “pharmaceutically acceptable” refers to a material that is not biologically or otherwise unacceptable, that can be incorporated into a composition and administered to a patient without causing unacceptable biological effects or interacting in an unacceptable manner with other components of the composition.

As used herein, the phrase “pharmaceutically acceptable salt” means a salt prepared from a base or an acid which is acceptable for administration to a patient, such as a mammal (e.g., salts having acceptable mammalian safety for a given dosage regime).

As used herein, the term “salt” can include salts derived from pharmaceutically acceptable inorganic acids and bases and salts derived from pharmaceutically acceptable organic acids and bases and their derivatives and variants thereof.

As used herein, the term “sample” refers to any system that haplomers can be administered into, where nucleic acid templated assembly may occur. Examples of samples include, but are not limited to, fixed or preserved cells, whole organisms, tissues, tumors, lysates, or in vitro assay systems.

As used herein, the phrases “set of corresponding reactants” or “corresponding haplomers” refer to haplomers that come together on a single target nucleic acid molecule to take part in a templated assembly reaction.

As used herein, the phrase “target compartment” refers to a cell, virus, tissue, tumor, lysate, other biological structure, spatial region, or sample that contains target nucleic acid molecule(s), or a different amount of target nucleic acid molecules than a non-target compartment.

As used herein, the phrases “target nucleic acid sequence” and “target nucleic acid molecule” are used interchangeably and refer to a sequence of units or nucleic acids which are intended to act as a template for nucleic acid templated assembly.

As used herein, the phrase “templated assembly product,” refers to the protein formed by two fragments of a particular protein associated with the haplomers.

As used herein, the phrase “traceless bio-orthogonal chemistry” refers to a reaction involving haplomers in which a naturally occurring bond, such as an amide, is formed by elimination of part or all of the bio-orthogonal moiety from the structure thus produced.

Nucleic acid molecules that are specific to designated cells of interest (whether these are represented by pathological tumor cells, abnormal immune cells, or any other cellular types) can be used as templates for the generation of novel structures (e.g., effector structures) by means of proximity-induced enhancement of molecular interactions (see, for example, PCT Publication No. WO 2014/197547). Such templated products can be designed to trigger cell death in various ways, or to modulate cellular activities. Cell-type specific nucleic acids can be sourced from specific transcribed mRNAs, or via nucleic acid aptamers which can serve to adapt non-nucleic acid targets for the provision of a defined template sequence.

In the original process of templated assembly for diagnostic or therapeutic purposes described above, reactive groups are brought into spatial proximity by virtue of their linkage with oligonucleotides of predetermined sequence, which themselves co-hybridize in proximity on a target nucleic acid molecule template. The template-directed modified oligonucleotides bearing mutually reactive groups are termed “haplomers.” Such enforced proximity of reactive groups greatly enhances product formation, and thus cell-type specific transcripts can direct the production of desired molecules in cells of interest. The general principle of TAPER can be altered to a two-level process, as described herein, by appending specific ligands to each haplomeric oligonucleotide instead of directly interactive functional groups. Thus, in the original configuration of TAPER (herein termed “conventional TAPER”), the process can be signified as occurring within a single reaction sequence, where the template can be considered functionally as a specific catalyst:

$$\begin{matrix}( Equation 1 ) & \\ H 1 - A + H 2 - B {\to }^{Template} H 1 - [ A : B ] - H 2 ⟶ H 1 - [ P ] - H 2 & ( 1 )\end{matrix}$$

where H1 and H2 represent haplomers bearing reactive groups A and B, respectively. Upon hybridization to specific template, a proximity-driven reaction intermediate between A and B is formed [A:B], leading rapidly to the formation of product [P].

In some embodiments, the variation of TAPER referred to as “locked TAPER” is readily applicable to SP-TAPER. For locked SP-TAPER, the first bottle haplomer and second haplomer interact as described herfein. By the nature of the locked TAPER process, the hybridization site for the second haplomer-protein fragment conjugate is not accessible except in the presence of specific target, where hybridization occurs with the anti-target loop portion of the first bottle haplomer. Subsequently, the hybridization site for the second haplomer-protein fragment conjugate is rendered accessible, and in turn proximity-promoted assembly.

The SP-TAPER processes and components thereof can be generally described by the following general representations.

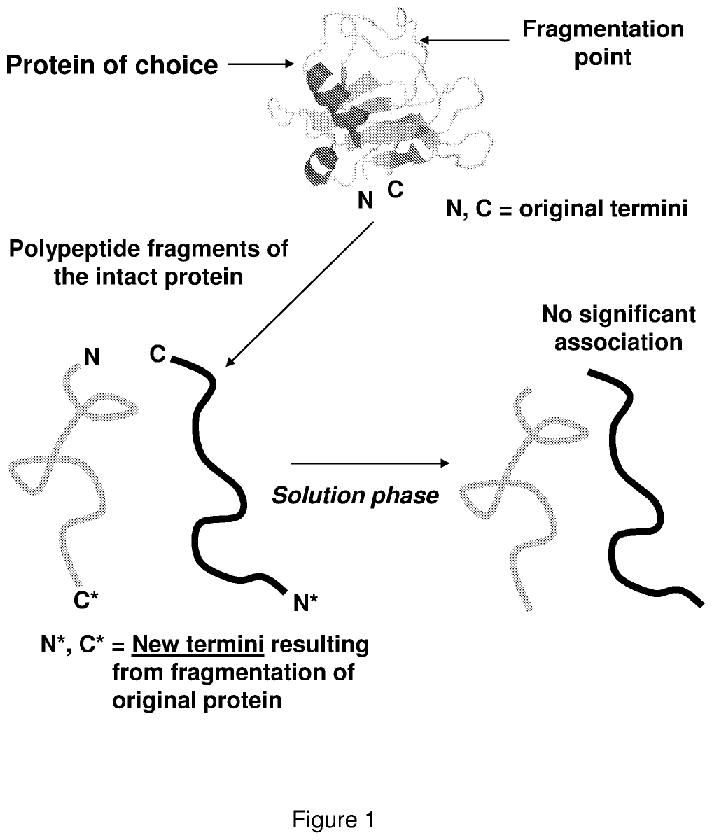

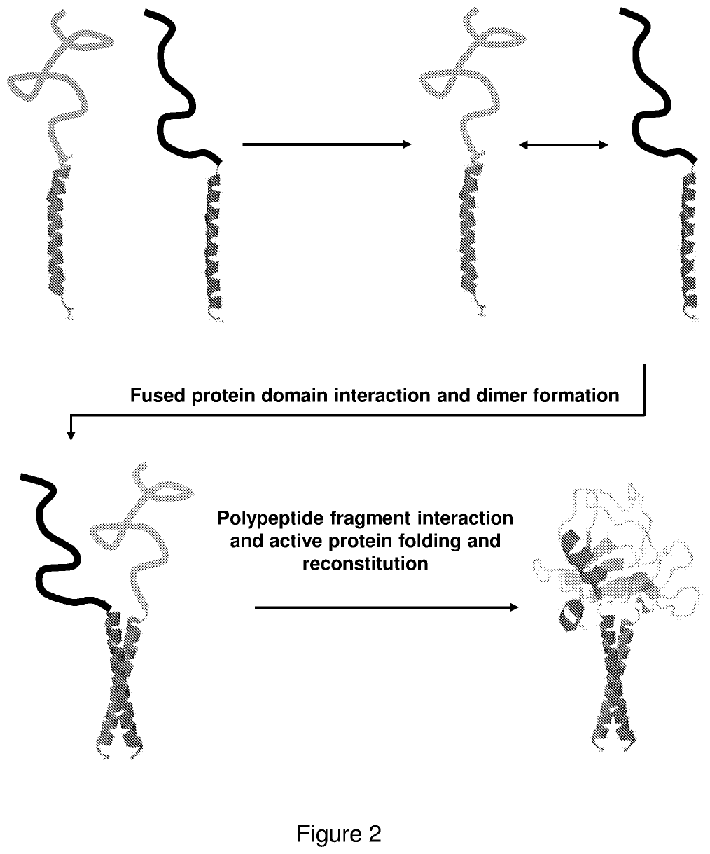

Numerous proteins can be divided into two separate polypeptide fragments that are disordered in isolation, but which can undergo accurate co-folding when held together in the correct orientation in spatial proximity. Such spatially enforced folding can result in the formation of the mature protein, including reconstitution of its original functional properties. One means for eliciting spatial proximity between such protein fragments has been to append each to independently folding and mutually interactive small protein domains, such as leucine zippers. This process has commonly been called the Protein Complementation Assay (PCA), or split-protein technology, and is depicted schematically in FIGS. 1 and 2. The specific choice of a site within a primary amino acid sequence for division of a protein of interest can be rationally guided when the protein three-dimensional structure is available. Loops or other structural features which can be modified without compromising general protein folding or function are accordingly favored for split-protein procedures. The spatial orientation of N- and C-termini of proteins of interest may also be significant. For example, where the N- and C-termini are packed in spatial proximity in the mature folded protein (see, FIG. 1), a parallel orientation of these termini in split-protein complementation may be more compatible with the required folding pathway than an anti-parallel orientation. Nevertheless, such potential constraints may be reduced or eliminated if each fragment is equipped with a flexible linker sequence of sufficient length to allow spatial positioning. Where no other information exists regarding the utility of a chosen fragmentation point for split-protein analyses, the system may be empirically tested by separately expressing fragments as appropriate fusions with self-folding and interactive protein domains, and testing reconstitution of functional activity upon mixing of fragments in vitro, or co-expressing the fusion products intracellularly. As an example of one such arrangement, a protein rendered as two fragments A and B is engineered to be expressed separately as A-(C-terminal)-Jun and Fos-(N-terminal)-B, where Jun and Fos are derived from c-Jun and c-Fos mutually interactive leucine zippers, and where long serine-glycine linkers separate the Jun/Fos segments from the desired polypeptide.

As described herein, it has been shown that nucleic acid hybridization can substitute for mutually interactive protein domains for the purposes of generating the spatial proximity between split-protein fragments for functional reconstitution. The protein fragments have been conjugated in suitable orientations with mutually complementary oligonucleotides, for diagnostic, imaging, and therapeutic purposes.

The use of nucleic acid templates to enforce molecular proximity and concomitant bimolecular reactions has been applied for the assembly of peptides or other small molecules in a therapeutic or diagnostic context (referred to as the TAPER process, where participating modified nucleic acids are termed Haplomers™ (see, PCT Publication WO 14/197547). Although previous descriptions of this process have focused on the assembly of small molecules, there is no inherent size restriction on the nature of the assembled molecular species. The present embodiments use TAPER and haplomer technology for the directed assembly of polypeptides by means of split-protein approaches. As such, such applications are classified as subsets of the TAPER process, and are collectively termed Split-Protein TAPER, or SP-TAPER. The use of TAPER as previously described is termed herein “conventional TAPER.” Oligonucleotides conjugated with protein fragments for SP-TAPER are herein referred as “SP-haplomers,” by extension from conventional TAPER.

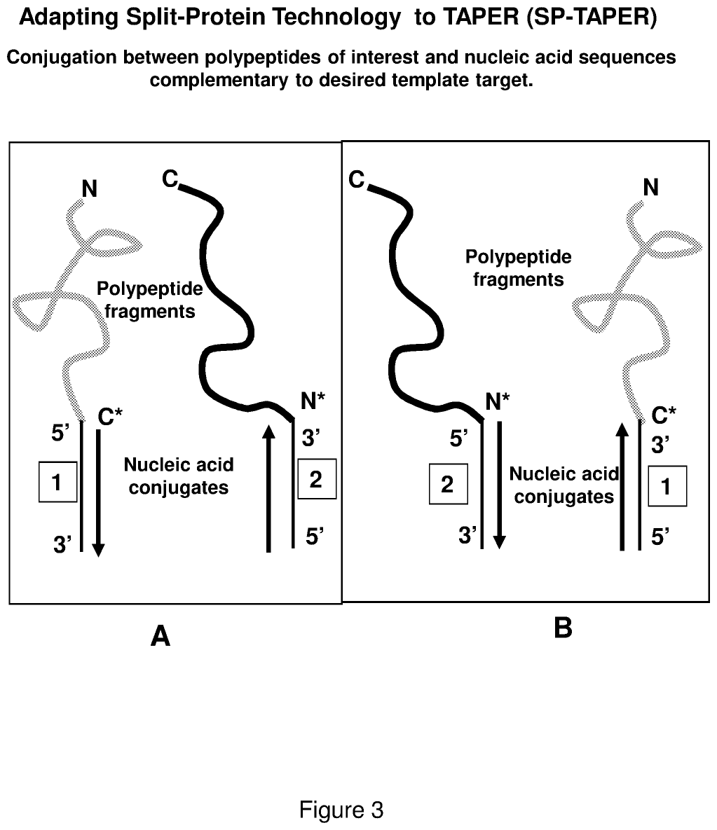

In order to adapt conventional TAPER to SP-TAPER, protein fragments are coupled with nucleic acid haplomers, whereby the haplomers enable hybridization-mediated molecular proximity between the two protein fragments. These haplomers are appended to the new N- or C-termini generated by expression of the protein of choice as two separate fragments (herein, these new termini are referred to as N*- and C*-respectively). The 5′ or 3′ ends of an oligonucleotide can be appended to either the N*- and C*-termini of split-protein fragments (see, FIG. 3) by various chemistries (panel A vs. panel B).

Prior to nucleic acid conjugations, protein fragments of interest are expressed in bacterial systems and purified. In some embodiments, expression systems include, but are not limited to, affinity fusions with maltose-binding protein, or hexahistidine tags. In some embodiments, intein fusions are expressed such that the desired protein fragments are cleaved off in vitro under appropriate conditions.

In some embodiments, the coupling between haplomers and protein fragments is mediated by bridging terminal —SH groups. For oligonuncleotides, 5′ or 3′ —SH groups are readily created by syntheses, where the sulfhydryl group is typically generated from a terminal precursor disulfide immediately prior to use, by treatment with reducing agents such as dithiothreitol (DTT) or TCEP. For polypeptides, N- or C-terminal —SH groups are most simply generated by placing a terminal cysteine residue at the appropriate site. Joining of —SH tagged oligonucleotides can be effected by means of bifunctional maleimide reagents including, but not limited to, 1,8-bis(maleimido)diethylene glycol and 1,11-bis(maleimido)triethylene glycol. The presence of internal cysteines within the polypeptide fragments of interest is a potential hurdle of this approach, but in practice it has been found that terminal cysteines are much more efficiently modified than those embedded within a longer sequence.

In some embodiments, the coupling between haplomers and protein fragments is mediated by alternative chemistries. For N-terminal polypeptide conjugations with haplomers, these approaches include, but are not limited to, ketene chemistry, thioazolidines, or isocyanato chelates. For C-terminal polypeptide conjugations with haplomers, these approaches include, but are not limited to, iodinylation of engineered C-terminal selenocysteines, and methods where labeling is coupled with intein cleavage. In the latter circumstances, cleavage of an N-terminal protein fragment of interest from a fused intein sequence can be effected by means of a hydrazino compound bearing an azido group (Kalia, et al., Chem. BioChem., 2006, 7, 1375-1383). Subsequent to this, an oligonucleotide carrying a 5′ or 3′ cyclooctyne group can be readily joined to the azide moiety through copper-free click chemistry. Alternately, an N-terminal protein fragment of interest can be cleaved from a fused intein sequence by conventional treatment with 2-mercaptoethane sulfonic acid, while co-reacted with a novel modified cysteine bearing a methyltetrazine group ((R)-2-amino-3-mercapto-N-(3-(4-(6-methyl-1,2,4,5-tetrazin-3-yl)phenoxy)propyl)propanamide):

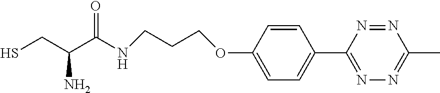 [SMILES: Cc1nnc(-c2ccc(OCCCNC(=O)[C@@H](N)CS)cc2)nn1]

This combines release of the desired N-terminal protein fragment with conjugation of the cysteine-methytetrazine. In turn, an oligonucleotide carrying a 5′ or 3′ trans-cyclooctene group reacts rapidly and specifically with the appended methytetrazine moiety.

In some embodiments, the coupling between haplomers and protein fragments is mediated through an extended genetic code. To implement this, a TAG stop codon (at the DNA level) is engineered at an N- or C-terminal position, and the bacterial strain used for expression purposes is co-transfected with plasmids encoding a bio-orthogonal aminoacyl tRNA synthase/tRNA pair, derived from an archaeal source with specific sequence modifications. In such circumstances, the aminoacyl tRNA synthase has been engineered and selected to bio-orthogonally charge its cognate tRNA with the desired unnatural amino acid, which is incorporated into proteins in a site-specific manner by virtue of the recognition of UAG codons by the tRNA anticodon triplet. In some instantiations of the extended genetic code approach, the unnatural amino bears a click group, including, but not limited to, trans-cyclooctene. When an unnatural amino acid residue with a side-chain bearing a specific click group is incorporated at or near the N- or C-terminus of a polypeptide of choice, an oligonucleotide bearing a reaction-complementary click group can be chemically ligated to the polypeptide via the particular click reaction itself. In the embodiment where the incorporated unnatural amino acid carries a trans-cyclooctene, bio-orthogonally reactive oligonucleotides are appended with a 5′ or 3′ methyltetrazine group.

Protein fragments conjugated with polynucleotides of haplomers may be purified from other proteins and unconjugated excess nucleic acids that are present. Purification methods include, but are not limited to, dialysis (where substantial molecular weight differences exist between the conjugate of interest and other components), gel filtration, non-denaturing gel electrophoresis and specific band excision, and HPLC.

In some embodiments, where the haplomers appended to polypeptides are composed of DNA, conjugates may be purified by hybridization with a biotinylated complementary RNA strand and subsequent immobilization on solid-phase streptavidin. Components of the initial mixture which lack DNA oligonucleotide haplomers are not bound to the solid-phase streptavidin, and are therefore removed by washing steps. Bound conjugates are then released by treatment with RNaseH, which specifically digests the RNA strand in RNA:DNA hybrids.

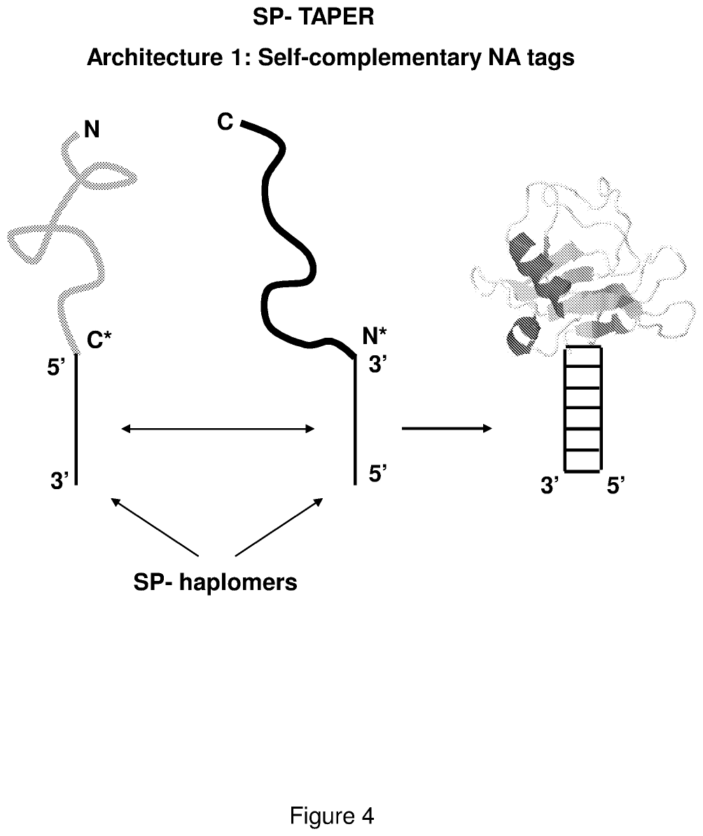

SP-TAPER may be instituted where the hybridization-mediated polypeptide juxtaposition (that enables folding and functional activity reconstitution) occurs by means of a number of distinct molecular architectures. In the simplest arrangement, the haplomers on each split-protein fragments are substantially complementary to each other. Direct hybridization between such haplomers promotes spatial proximity of the appended protein fragments, and in turn their co-interaction via the native folding pathway (see, FIG. 4). Herein, this configuration is referred to as “Architecture 1.”

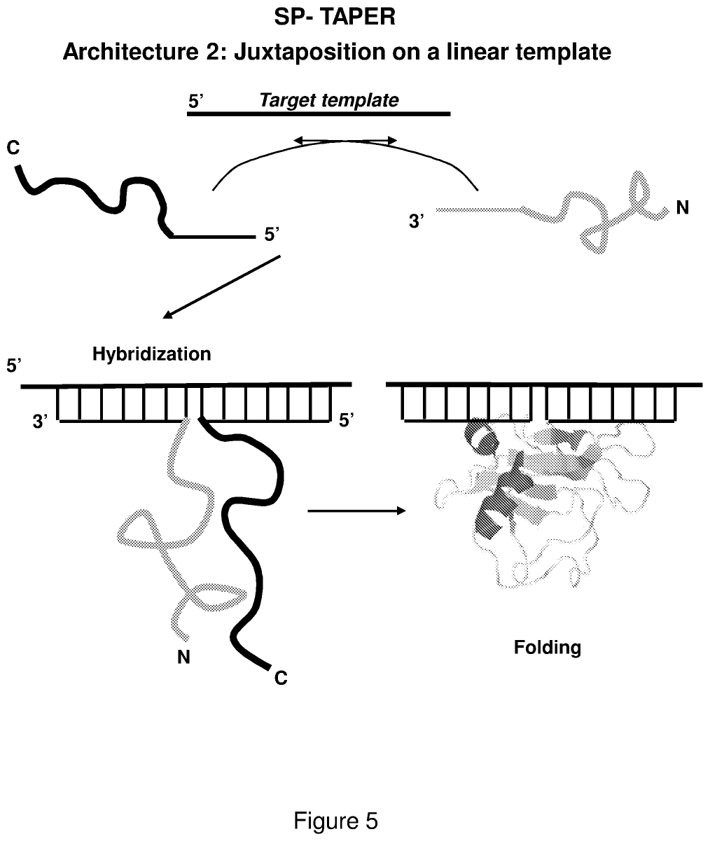

To closely parallel conventional TAPER, a pair of SP-haplomers can also co-hybridize in spatial proximity to a third-party linear target template, rather than being complementary to each other. By so doing, the appended polypeptide sequences are arranged in spatial juxtaposition in the desired orientation, such that the mature folded protein product can form (see, FIG. 5). Herein, this configuration is referred to as “Architecture 2.” Within this architecture, the gap between the two hybridizing SP-haplomers on a complementary template (i.e., target nucleic acid molecule) may be zero (that is, when the SP-haplomers are precisely juxtaposed) or withN>0, where N=the number of template nucleotides between the 5′ and 3′ ends of the SP-haplomer pair. (In practice, as N increases, the efficiency of interaction between haplomer polypeptides will tend to diminish).

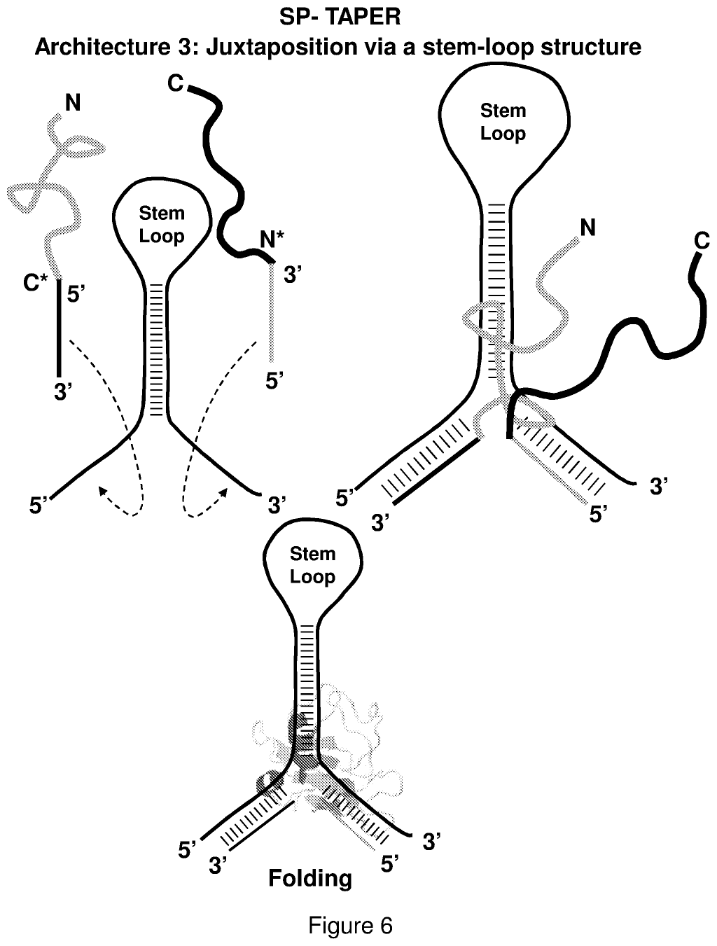

Additional architectures are possible for SP-TAPER, where the sites of hybridization of SP-haplomers are non-contiguous in terms of the primary sequence of the target template. Where discontinous recognition sites for the pair of SP-haplomers are brought into spatial proximity by a stem-loop structure (herein, termed “Architecture 3”), the appended polypeptide sequences can co-fold into the mature protein structure (see, FIG. 6).

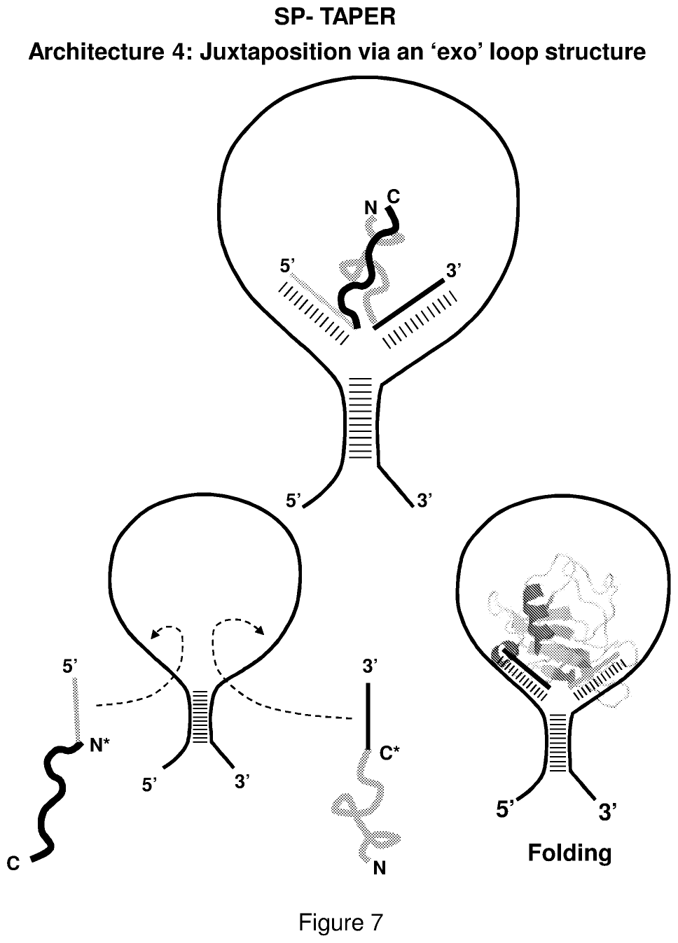

In the template-based Architectures 2 and 3, the 5′ and 3′ of the SP-haplomers are directed towards each other in terms of the coordinates of the template strand. This has previously been termed an “endo” configuration. Where the template strand can form a sizeable loop structure, opposite haplomer arrangement (“exo” configuration, with the 5′ and 3′ of the SP-haplomers directed away each other in terms of the coordinates of the template strand (see FIG. 7) can also result in spatial proximity of the appended polypeptide segments, along with functional co-folding. Herein, this configuration is referred to as “Architecture 4.”

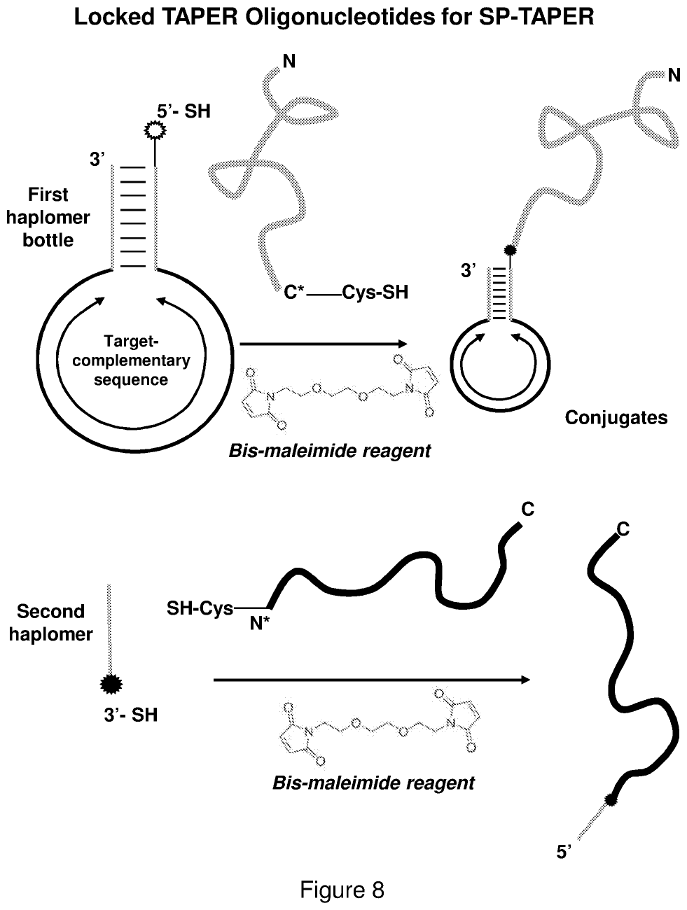

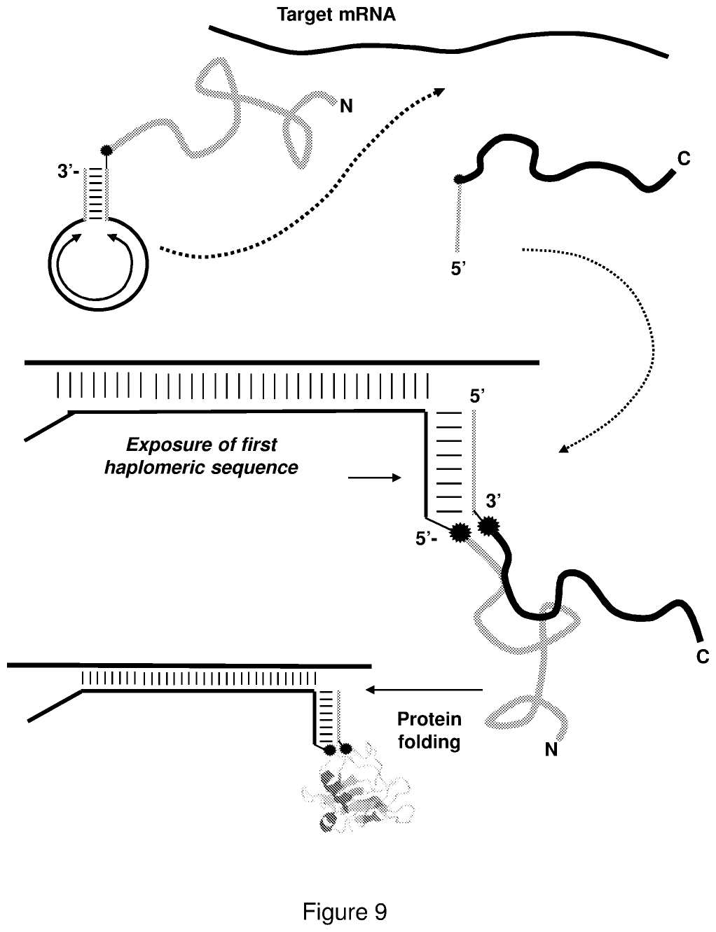

In some embodiments, the variation of TAPER previously referred to as “locked TAPER” (i.e., TAPER processes using a bottle haplomer) is readily applicable to SP-TAPER. The use of locked TAPER helps circumvent any template titration effects. For locked SP-TAPER, the first haplomer bottle and second haplomers are conjugated with predetermined and independently expressed polypeptide fragments of a protein of interest, in an analogous manner to other SP-TAPER architectures in the above embodiments. The conjugation process between the first haplomer bottle and second haplomers with the polypeptide fragments of interest can be achieved in various ways corresponding to the embodiments above, including, but not limited to, thiol conjugations by means of a bifunctional maleimide coupling reagent (see, FIG. 8). By the nature of the locked TAPER process, the hybridization site for the second haplomer-polypeptide conjugate is not accessible except in the presence of specific target nucleic acid molecule, where hybridization occurs with the anti-target loop portion of the first haplomer bottle. Subsequently, the the hybridization site for the second haplomer-polypeptide conjugate is rendered accessible, and in turn the proximity-promoted co-folding of the two polypeptide chains can ensue (see, FIG. 9).

Within a locked-TAPER system, when the two oligonucleotides bearing polypeptide conjugates are in hybridization-mediated spatial proximity (see, FIG. 9), the structure of the assembly pieces corresponds to Architecture 1 (see FIG. 9 and FIG. 4), since the two derivatized oligonucleotides are complementary to each other, rather than complementary to a target template as in Architectures 2-4. Nevertheless, since the loop section of a locked-TAPER first haplomer bottle hybridizes to a target nucleic acid molecule sequence in order to expose the recognition site for the second haplomer, the loop-target binding itself can occur via different architectures. Thus, the target hybridization of the locked TAPER oligonucleotide in FIG. 9 corresponds to Architecture 2 (see, FIG. 5), and target hybridization by means of Architectures 3 and 4 (see, FIGS. 6 and 7, respectively) are equally possible. Locked TAPER accordingly has the unique feature whereby the TAPER assembly is always constant with Architecture 1, but target hybridization can assume variable architectures. In other words, for conventional TAPER, the target hybridization and assembly-directing hybridizations coincide, but for locked TAPER they are distinct and separable.

Proteins that can be applied as split polypeptides towards templated reassembly directed by SP-TAPER include all those capable of delivering a reporter signal. A non-limiting set of reporter protein examples includes fluorescent proteins (such as GFP and derivatives, YFP, mCherry, dsRed, VENUS, and CFP), and luciferases (firefly luciferase, Renilla luciferase). Other classes of proteins encompassed by SP-TAPER applications include, but are not limited to, transcription factors, signal transduction pathway factors, and gene editing proteins.

In some embodiments, SP-TAPER is targeted towards the templated assembly of single-chain immunoglobulin variable region (scFv) proteins. These typically contain extended serine-glycine linker sequences which enable the association of the variable region heavy and light chain segments. This linker sequence is a convenient site for split protein generation, where the two immunoglobulin variable region segments are appended with nucleic acid tags according to the desired architecture (see, FIGS. 5-8), after which their assembly and resulting antigen-binding properties are mediated by the presence of specific template. This enables in situ generation of a desired antigen-binding specificity in a cell target of interest, as defined by a cell-specific nucleic acid sequence. Applications of scFv-targeted SP-TAPER include, but are not limited to, the use of fluorescence-activating proteins (FAPs, scFv molecules generating fluorescence in target ligands).

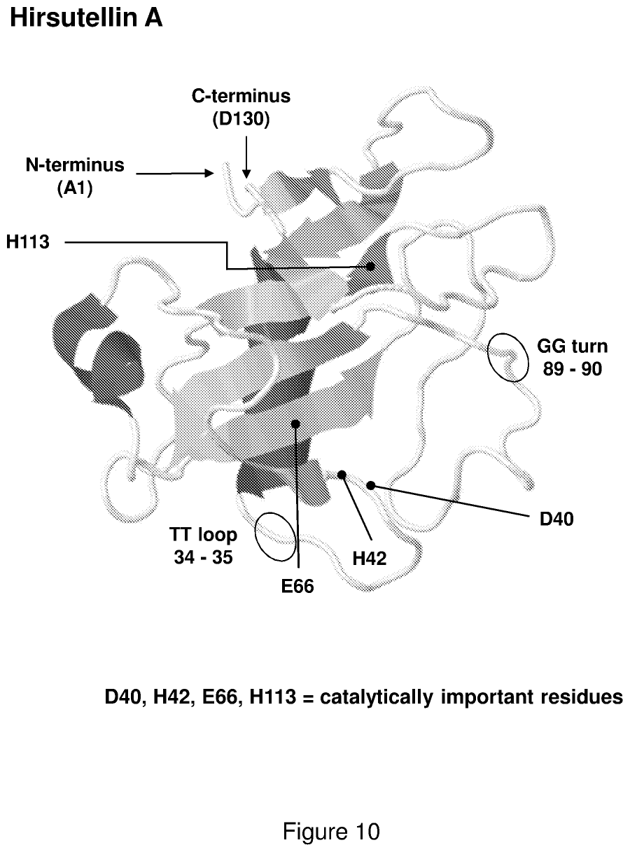

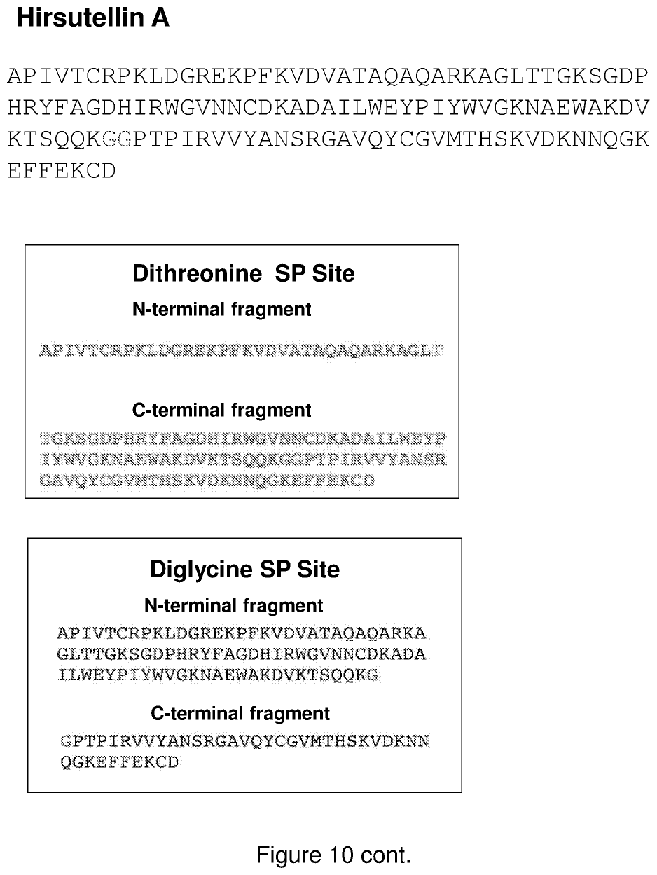

In some embodiments, SP-TAPER is applied towards the templated assembly of small highly toxic proteins, or ribotoxins, which function by enzymatically disabling eukaryotic ribosomes. Such proteins include, but are not limited to, ricin A chain, Aspf1, α-sarcin, mitogillin, and hirsutellin A. These proteins are attractive for SP-TAPER through their small sizes and high toxicities. Hirsutellin A, as a non-limiting example, has a number of potential split-protein fragmentation sites, including a diglycine turn (see, FIG. 10). While extreme toxicity can be a significant restriction on the deployment of ribotoxins as direct immunoconjugates (through unacceptable by-stander effects) this is effectively circumvented by SP-TAPER. Where ribotoxin split-protein fragments lack the toxic activity of their parental protein, their circulating fragments are innocuous. With SP-TAPER, such fragments only assemble into fully active proteins in the presence of specific templates associated with a pathological cell target.

By the same principles as noted for ribotoxins, SP-TAPER is also applicable to the template-directed assembly of other small and highly toxic proteins, including, but not limited to, diphtheria toxin and cholera toxin. Further examples are provided below.

Target nucleic acid molecules that serve as templates for SP-TAPER include any nucleic acid sequence which distinguishes a target of choice, whether the sequence corresponds to a cellular RNA molecule of any description, or derives from an aptamer-mediated adaptation process (see, U.S. Ser. No. 62/339,981), or from any other process whereby a suitable template sequence is afixed at a desired cellular locale.

Target nucleic acid molecules that serve as templates for SP-TAPER may be produced on cell surfaces, where the template-promoted assembly of SP-haplomers is also a surface effect. In some embodiments, the specific desired templates (and desired split-protein assembly sites) are internally situated within a target cell type, whether of tumor origin, arising through aberrant immune pathways, or originating by means of any other type of pathological process. In such cases, the SP-haplomers are dispatched to the intracellular environment by various delivery technologies including, but not limited to, gymnotic approaches, and a wide variety of nanoparticles. The latter category includes, but is not limited to, simple and multi-layered liposomes, dendrimers, extracellular vesicles, DNA or other nucleic acid origami cages, engineered bacterial vehicles, engineered mitochondria, virally-derived structures, ribonucleoprotein vaults, and protein or PEGylated protein self-assembling compartments. As with conventional TAPER, while target precision of delivery is useful, it is not essential, since in the absence of the pathologically-defined target sequence, no split-protein assembly will take place. In other words, delivery to a normal “off-target” cell does not have deleterious side-effects for the implementation of SP-TAPER.

In some embodiments, the folding pathway of the split polypeptide fragments in SP-TAPER may be assisted by the provision of protein chaperones (including, but not limited to, members of diverse heat-shock protein families), or low-molecular weight chemical chaperones. Small-molecule chaperones in the latter category with non-specific chaperoning function include, but are not limited to, 4-phenyl butyrate, deoxycholic acid, ursodeoxycholic acid, or taurourso-deoxycholic acid. In some embodiments, SP-TAPER may utilize small molecules that have beneficial folding enhancement towards specific target polypeptide fragments of interest, where such low-molecular weight compounds are defined as pharmacological chaperones, or pharmacoperones.

The SP-TAPER processes and components thereof can be generally described by the following more specific embodiments.

The present disclosure provides haplomers comprising: a) a polynucleotide that is substantially complementary to a target nucleic acid molecule; and b) an N-terminal protein fragment or a C-terminal protein fragment, wherein the 3′ or 5′ terminus of the polynucleotide is linked to the N-terminus of the C-terminal protein fragment or the C-terminus of the N-terminal protein fragment. In some embodiments, the polynucleotide of the haplomer comprises from about 6 to about 20 nucleotide bases. In some embodiments, the the polynucleotide of the haplomer comprises from about 8 to about 15 nucleotide bases.

In some embodiments, a pair of haplomers works in tandem. In some embodiments, the protein fragment of the first haplomer is linked to the 5′ terminus of the polynucleotide of the first haplomer, and the protein fragment of the second haplomer is linked to the 3′ terminus of the polynucleotide of the second haplomer.

In some embodiments, the polynucleotide of the first haplomer is substantially complementary to the polynucleotide of the second haplomer. In some embodiments, the polynucleotide of the first haplomer is substantially complementary to a target nucleic acid molecule, and the polynucleotide of the second haplomer is substantially complementary to the target nucleic acid molecule at a site in spatial proximity to the polynucleotide of the first haplomer.

In any of the embodiments described herein, the haplomers are in spatial proximity (when bound to a target nucleic acid molecule) such that the protein fragments can properly interact to induce the interaction of their respective fragments of the protein of interest. Thus, for any haplomer pairs, reactivity can occur where the gap N between the first and second haplomer binding to the target nucleic acid molecule is 0 (i.e., the haplomers are immediately juxtaposed), and progressively greater gaps (N>0) will progressively diminish activity. Thus, in some embodiments, there is 0 nucleotides between the binding of a first haplomer and second haplomer to the target nucleic acid molecule. In some embodiments, there is less than 6 nucleotides between the binding of a first haplomer and second haplomer to the target nucleic acid molecule. In some embodiments, there is less than 5 nucleotides between the binding of a first haplomer and second haplomer to the target nucleic acid molecule. In some embodiments, there is less than 4 nucleotides between the binding of a first haplomer and second haplomer to the target nucleic acid molecule. In some embodiments, there is less than 3 nucleotides between the binding of a first haplomer and second haplomer to the target nucleic acid molecule. In some embodiments, there is less than 2 nucleotides between the binding of a first haplomer and second haplomer to the target nucleic acid molecule.

In some embodiments, the protein fragment and polynucleotide of the first haplomer both comprises reactive bio-orthogonal moieties, and/or the protein fragment and polynucleotide of the second haplomer both comprises reactive bio-orthogonal moieties, wherein the reactive bio-orthogonal moiety of the first haplomer is reactable with the bio-orthogonal moiety of the second haplomer.

In some embodiments, the N-terminal fragment comprises the amino acid sequence of APIVTCRKLDGREKPFKVDVATAQAQARKAGLTITGKSGDPHRYFAGDHIRWGVNNCD KADAILWEYPIYWVGKNAEWAKDVKTSQQKG (SEQ ID NO:1), and the C-terminal fragment comprises the amino acid sequence of GPTPIRVVYANSRGAVQYCGVMTHSKVD KNNQGKEFFEKCD (SEQ ID NO:2).

In some embodiments, the N-terminal fragment comprises the amino acid sequence of APIVTCR PKLDG (SEQ ID NO:3), and the C-terminal fragment comprises the amino acid sequence of REKPFKVDVATAQAQARKAGLTTGKSGDPHRYFAGDHIRWGVNNCDKAD AILWEYPIYWVGKNAEWAKDVKTSQQKGGPTPIRVVYANSRGAVQYCGVMTHSKVD KNNQGKEFFEKCD (SEQ ID NO:4).

In some embodiments, the N-terminal fragment comprises the amino acid sequence of APIVTCRPKLDGREKPFKVDVATAQAQARKAGLTTGK (SEQ ID NO:5), and the C-terminal fragment comprises the amino acid sequence of SGDPHRYFAGDHIRWGVNNCD KADAILWEYPIYWVGKNAEWAKDVKTSQQKGGPTPIRVVYANSRGAVQYCGVMTHS KVDKNNQGKEFFEKCD (SEQ ID NO:6).

In some embodiments, the N-terminal fragment comprises the amino acid sequence of APIVTCRPKLDGREKPFKVDVATAQAQARKAGLTTGKSGDPHRYFAGDHIRWGVNNC DKAD (SEQ ID NO:7), and the C-terminal fragment comprises the amino acid sequence of AILWEYPIYWVGKNAEWAKDVKTSQQKGGPTPIRVVYANSRGAVQYCGVMTHSKVD KNNQGKEFFEKCD (SEQ ID NO:8).

In some embodiments, the N-terminal fragment comprises the amino acid sequence of APIVTCRPKLDGREKPFKVDVATAQAQARKAGLTTGKSGDPHRYFAGDHIRWGVNNC DKADAILWEYPIYWVG (SEQ ID NO:9), and the C-terminal fragment comprises the amino acid sequence of KNAEWAKDVKTSQQKGGPTPIRVVYANSRGAVQYCGVMTHSKVDK NNQGKEFFEKCD (SEQ ID NO:10).

In some embodiments, the N-terminal fragment comprises the amino acid sequence of APIVTCRPKLDGREKPFKVDVATAQAQARKAGLTTGKSGDPHRYFAGDHIRWGVNNC DKADAILWEYPIYWVGKNAEWAKD (SEQ ID NO: 11), and the C-terminal fragment comprises the amino acid sequence of VKTSQQKGGPTPIRVVYANSRGAVQYCGVMTHS KVDKNNQGKEFFEKCD (SEQ ID NO:12).

In some embodiments, the N-terminal fragment comprises the amino acid sequence of APIVTCRPKLDGREKPFKVDVATAQAQARKAGLTTGKSGDPHRYFAGDHIRWGVNNC DKADAILWEYPIYWVGKNAEWAKDVKTSQ (SEQ ID NO:13), and the C-terminal fragment comprises the amino acid sequence of QKGGPTPIRVVYANSRGAVQYCGVMTHSK VDKNNQGKEFFEKCD (SEQ ID NO:14).

In some embodiments, the N-terminal fragment comprises the amino acid sequence of APIVTCRPKLDGREKPFKVDVATAQAQARKAGLTTGKSGDPHRYFAGDHIRWGVNNC DKADAILWEYPIYWVGKNAEWAKDVKTSQQKGGPTPIRVVYANSRG (SEQ ID NO:15), and the C-terminal fragment comprises the amino acid sequence of AVQYCGVMTHSKVDKN NQGKEFFEKCD (SEQ ID NO:16).

In some embodiments, the N-terminal fragment comprises the amino acid sequence of APIVTCRPKLDGREKPFKVDVATAQAQARKAGLTTGKSGDPHRYFAGDHIRWGVNNC DKADAILWEYPIYWVGKNAEWAKDVKTSQQKGGPTPIRVVYANSRGAVQYCGVMTH SKVDKN (SEQ ID NO:17), and the C-terminal fragment comprises the amino acid sequence of NQGKEFFEKCD (SEQ ID NO:18).

In some embodiments, the N-terminal fragment comprises the amino acid sequence of APIVTCRPKLDGREKPFKVDVATAQAQARKAGLT; (SEQ ID NO:40), and the C-terminal fragment comprises the amino acid sequence of TGKSGDPHRYFAGDHIRWGVNNCDKAD AILWEYPIYWVGKNAEWAKDVKTSQQKGGPTPIRVVYANSRGAVQYCGVMTHSKVD KNNQGKEFFEKCD (SEQ ID NO:41).

The present disclosure also provides bottle haplomers comprising a polynucleotide, wherein the polynucleotide comprises: a) a first 3′ stem portion comprising from about 10 to about 20 nucleotide bases; b) an anti-target loop portion comprising from about 16 to about 40 nucleotide bases linked to the first 3′ stem portion, wherein the anti-target loop portion is substantially complementary to a target nucleic acid molecule; and c) a second 5′ stem portion comprising from about 10 to about 20 nucleotide bases linked to the anti-target loop portion, wherein the first 3′ stem portion is substantially complementary to the second 5′ stem portion; wherein: i) the 5′ terminus of the polynucleotide comprises a —SH moiety; and ii) the Tₘ of the anti-target loop portion:target nucleic acid molecule is greater than the Tₘ of the first stem portion:second stem portion.

The present disclosure also provides bottle haplomers comprising a polynucleotide, wherein the polynucleotide comprises: a) a first 3′ stem portion comprising from about 10 to about 20 nucleotide bases; b) an anti-target loop portion comprising from about 16 to about 40 nucleotide bases linked to the first 3′ stem portion, wherein the anti-target loop portion is substantially complementary to a target nucleic acid molecule; and c) a second 5′ stem portion comprising from about 10 to about 20 nucleotide bases linked to the anti-target loop portion, wherein the first 3′ stem portion is substantially complementary to the second 5′ stem portion; wherein: i) the Tₘ of the anti-target loop portion:target nucleic acid molecule is greater than the Tₘ of the first stem portion:second stem portion; and ii) the 5′ terminus or 3′ terminus of the polynucleotide is linked to the C-terminus of an N-terminal protein fragment or the N-terminus of a C-terminal protein fragment, wherein the terminus of the protein fragment lined to the polynucleotide comprises a cysteine or selenocysteine.

In some embodiments, the first stem portion comprises from about 12 to about 18 nucleotide bases. In some embodiments, the anti-target loop portion comprises from about 18 to about 35 nucleotide bases. In some embodiments, the second stem portion comprises from about 12 to about 18 nucleotide bases. The anti-target loop portion has a first end to which the first stem portion is linked. The anti-target loop portion is substantially complementary to a target nucleic acid molecule. The second stem portion is linked to a second end of the anti-target loop portion. The first stem portion is substantially complementary to the second stem portion.

In some embodiments, the anti-target loop portion can further comprise an internal hinge region, wherein the hinge region comprises one or more nucleotides that are not complementary to the target nucleic acid molecule. In some embodiments, the hinge region comprises from about 1 nucleotide to about 6 nucleotides, from about 1 nucleotide to about 5 nucleotides, from about 1 nucleotide to about 4 nucleotides, from about 1 nucleotide to about 3 nucleotides, or 1 or 2 nucleotides.

For the polynucleotides of the bottle haplomers described herein, the length of the particular polynucleotide or portion thereof is less important than the Tₘ of the duplex formed by the interaction of the polynucleotide, or portion thereof, with another nucleic acid molecule, or portion thereof. For example, the Tₘ of the duplex formed by the interaction of the anti-target loop portion with the target nucleic acid molecule (e.g., anti-target loop portion:target nucleic acid molecule) is greater than the Tₘ of the duplex formed by the interaction of the first stem portion with the second stem portion (e.g., first stem portion:second stem portion). In some embodiments, the Tₘ of the first stem portion:second stem portion subtracted from the Tₘ of the anti-target loop portion:target nucleic acid molecule is from about 10° C. to about 40° C. In some embodiments, the Tₘ of the first stem portion:second stem portion subtracted from the Tₘ of the anti-target loop portion:target nucleic acid molecule is from about 10° C. to about 20° C. In some embodiments, the Tₘ of the first stem portion:second stem portion is from about 40° C. to about 50° C. In some embodiments, the Tₘ of the anti-target loop portion:target nucleic acid molecule is from about 60° C. to about 80° C.

In addition, translating the Tₘ information above into specific lengths of the nucleic acid molecules described herein can also depend on the GC content of each nucleic acid molecule. For example, the length of a suitable HPV model target nucleic acid molecule is 30 bases (having a Tₘ of 70° C.), while that for the EBV model target nucleic acid molecule is only 21 bases (having a Tₘ of 69° C.), owing to its greater % GC.

In some embodiments, a bottle haplomer works in tandem with a second haplomer. In some embodiments, the bottle haplomer is any bottle haplomer described herein, and the second haplomer is any of the haplomers described herein. In some embodiments, the second haplomer comprises: a) a nucleotide portion comprising from about 6 to about 20 nucleotide bases that is substantially complementary to the stem portion of the bottle haplomer that is linked to the protein fragment of the bottle haplomer; and b) a protein fragment linked to the 5′ or 3′ terminus of the nucleotide portion of the second haplomer; wherein the Tₘ of the second haplomer:first or second stem portion linked to the protein fragment of the bottle haplomer is less than or equal to the Tₘ of the first stem portion:second stem portion of the bottle haplomer.

In some embodiments, the Tₘ of the duplex formed by the interaction of the second haplomer with either the first stem portion or the second stem portion, whichever stem portion is linked to the protein fragment (e.g., second haplomer:first or second stem portion linked to the protein fragment), is less than or equal to the Tₘ of the first stem portion:second stem portion. In some embodiments, the Tₘ of the duplex formed by the second haplomer and the first or second stem portion linked to the protein fragment subtracted from the Tₘ of the first stem portion:second stem portion is from about 0° C. to about 20° C. In some embodiments, the Tₘ of the duplex formed by the second haplomer and the first or second stem portion linked to the protein fragment subtracted from the Tₘ of the first stem portion:second stem portion is from about 5° C. to about 10° C. In some embodiments, the Tₘ of the duplex formed by the second haplomer and the first or second stem portion linked to the protein fragment is from about 30° C. to about 40° C.

This structural arrangement is designed such that in the absence of target nucleic acid molecule template, the locked first haplomer bottle does not significantly hybridize to its complementary second haplomer and, thus, template-directed product assembly is not promoted under such conditions. When the specific target nucleic acid molecule template is present, on the other hand, the bottle haplomer is “unlocked” by the formation of a more stable hybrid between the anti-target loop portion of the bottle haplomer and the target nucleic acid molecule itself. Once this occurs, the first stem portion of the bottle haplomer that is linked to the protein fragment is free to hybridize to the available second haplomer, with resulting proximity between the protein fragments on both.

The present disclosure also provides surface target compounds comprising: a) a template polynucleotide; and b) a peptide; wherein: i) the 5′ terminus of the polynucleotide is coupled to the N-terminus or C-terminus of the peptide, or the 3′ terminus of the polynucleotide is coupled to the N-terminus or C-terminus of the peptide; and ii) the peptide is a ligand for a cell-surface molecule.

In some embodiments, the ligand is a peptide hormone or a neuropeptide. Examples of peptide hormones include, but are not limited to, alpha-MSH, amylin, anti-Müllerian hormone, adiponectin, atriopeptide, human growth hormone, gonadotropin releasing hormone, inhibin, somatostatin, adrenocorticotropic hormone, vasopressin, vasoactive intestinal peptide, gastrin, secretin, gastric inhibitory polypeptide, motilin, hepcidin, renin, relaxin, ghrelin, leptin, lipotropin, angiotensin I, angiotensin II, bradykinin, calcitonin, insulin, glucagon, insulin-like growth factor I, insulin-like growth factor II, glucagon-like peptide I, pancreatic polypeptide, betatrophin, cholecystokinin, endothelin, erythropoietin, thrombopoietin, follicle-stimulating hormone, human chorionic gonadotropin, human placental lactogen, prolactin, prolactin releasing hormone, luteinizing hormone, thyroid-stimulating hormone, thyrotropin-releasing hormone, parathyroid hormone, and pituitary adenylate cyclase-activating peptide.

Examples of neuropeptides include, but are not limited to, neuropeptide Y, an endorphin, an encephalin, brain natriuretic peptide, tachykinin, cortistatin, galanin, orexin, and oxytocin.

In some embodiments, the polynucleotide comprises the nucleotide sequence AAGCC ACTGTGTCCTGAAGAAAAGCAAAGACATC (SEQ ID NO:20), and the peptide comprises the amino acid sequence SYSMEHFRWGKPVGGGSSGGGC (SEQ ID NO:21), SYSXEHFRW GKPVGGGSSGGGC (SEQ ID NO:22), CSGGGSSGGGSYSMEHFRWGKPV-NH₂ (SEQ ID NO:23), or CSGGGSSGGGSYSXEHFRWGKPV-NH₂ (SEQ ID NO:24), wherein X is norleucine and the F residue is D-phenylalanine.

The present disclosure also provides fusion proteins comprising: an N-terminal protein fragment, a fusion partner protein, and a purification domain, wherein the C-terminus of the N-terminal protein fragment is coupled to the N-terminus of the fusion partner protein, and the C-terminus of the fusion partner protein is coupled to the N-terminus of the purification domain; or an N-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the N-terminal protein fragment, wherein the N-terminal protein fragment comprises an N-terminal methionine and a C-terminal cysteine; or a C-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the C-terminal protein fragment, wherein the C-terminal protein fragment comprises an N-terminal cysteine.

In some embodiments, the fusion protein comprises an N-terminal protein fragment, intein, and a chitin-binding domain, wherein the C-terminus of the N-terminal protein fragment is coupled to the N-terminus of intein, and the C-terminus of intein is coupled to the N-terminus of the chitin-binding domain. In some embodiments, the fusion protein comprises an N-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the N-terminal protein fragment, wherein the N-terminal protein fragment comprises an N-terminal methionine and a C-terminal cysteine. In some embodiments, the fusion protein comprises a C-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the C-terminal protein fragment, wherein the C-terminal protein fragment comprises an N-terminal cysteine.

In some embodiments, the fusion protein comprises an N-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the N-terminal protein fragment, wherein the N-terminal protein fragment comprises the amino acid sequence

(SEQ ID NO: 25)
APIVTCRPKLDGREKPFKVDVATAQAQARKAGLTTGKSGDPHRYFAG
DHIRWGVNNCDKADAILWEYPIYWVGKNAEWAKDVKTSQQKGC.

In some embodiments, the fusion protein comprises a C-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the C-terminal protein fragment, wherein the C-terminal protein fragment comprises the amino acid sequence

(SEQ ID NO: 26)
CGPTPIRVVYANSRGAVQYCGVMTHSKVDKNNQGKEFFEKCD.

In some embodiments, the fusion partner protein is intein, a maltose-binding protein, glutathione-S-transferase, β-galactosidase, or Omp F.

In some embodiments, the cleavage site is an enterokinase cleavage site or a Factor Xa protease cleavage site. In some embodiments, the Factor Xa protease cleavage site is IEGR (SEQ ID NO:27).

In some embodiments, the purification domain is a chitin-binding domain or a hexahistidine tag.

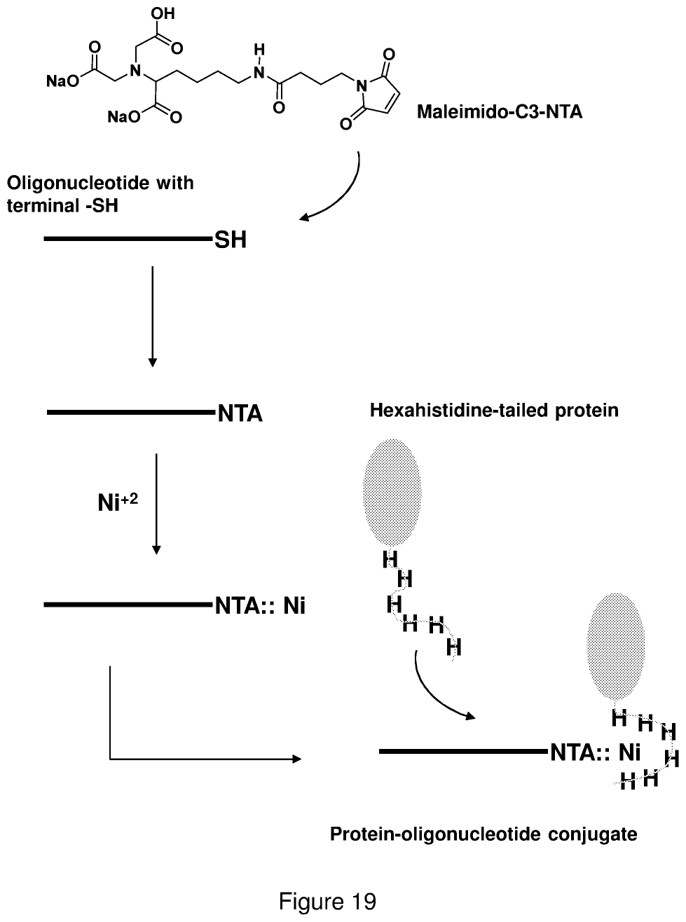

In some embodiments, coupling of oligonucleotides for SP-TAPER is effected by covalently modifying nucleic acid 5′ or 3′ termini with a chelating agent to enable oligonucleotide binding to hexahistidine split-protein fragment fusions. Oligonucleotides with 5′ or 3′ disulfide modifications are initially reduced with a molar excess of TCEP, and then run through desalting columns to purify the resulting thiol-terminal oligonucleotides from TCEP and low-molecular weight products. Following this, the free-thiol oligonucleotides are reacted with maleimido-C3-nitrilotriacetic acid (MNTA; Dojindo Molecular Technologies), such that the maleimide moiety of MNTA reacts with the available thiols to form a conjugate. This product is again purified from low-molecular species by desalting, and then is loaded with nickel ions by incubating with a molar excess of NiCl₂, and re-desalted to remove nickel excess. The resulting chelation conjugate can then be used to form a complex with split-protein fragments bearing either a C-terminal or N-terminal hexahistidine tag, produced by expression of appropriate coding sequences. The conjugation process is depicted in FIG. 19.

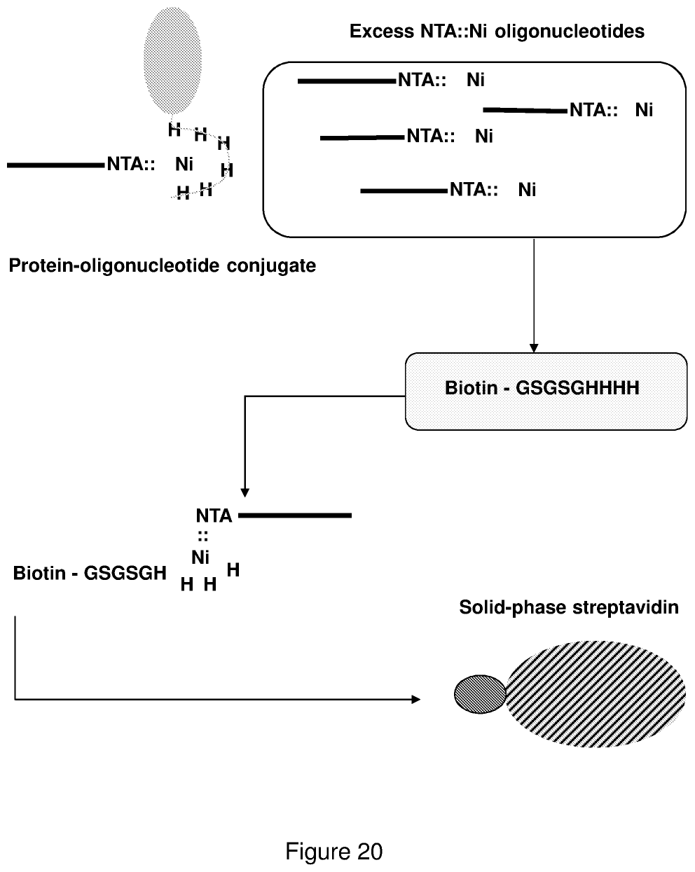

In some embodiments, excess NTA::Ni oligonucleotides can be removed from reactions forming complexes with hexahistidine, by means of a biotinylated oligonucleotide with a tetrahistidine sequence (biotin-GSGSGHHHH; SEQ ID NO:19). Since nickel chelates can still bind tetrahistidine but with reduced affinity relative to hexahistidine (Knecht et al., J. Molec. Recognition, 22: 270-279, 2009), excess tetrahistidine peptide can deplete unconjugated NTA::Ni oligonucleotides without competitively stripping oligonucleotides from the protein fragment histidine tag. The biotinylated peptide/oligonucleotide excess are then removed on solid-phase streptavidin preparations (see, FIG. 20). If necessary, the depletion step with the biotinylated tetrahistidine peptide can be repeated to remove residual unconjugated NTA::Ni oligonucleotide chelates.

Conjugates formed by complexing between NTA::Ni chelate and hexahistidine tags can be used in SP-TAPER in the same manner as for other chemical conjugation pathways, using any of the Architectures 1-4, and locked TAPER configurations (see, FIGS. 4-9).

The present disclosure also provides compounds having the formula

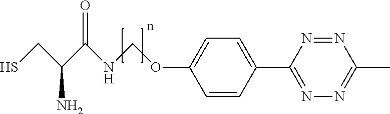

wherein n is from about 3 to about 6. In some embodiments, n is from about 4 to about 6 or from 5 to 6. In some embodiments, n is 3. In some embodiments, n is 4. In some embodiments, n is 5. In some embodiments, n is 6. In some embodiments, the compound is modified by replacing one or more hydrogens with various substituents including, for example, —OH, —C₁-C₆alkyl, —C₁-C₆alkenyl, and a halogen, and the like.

In any of the polynucleotides described herein, or any portion thereof, the nucleotide bases are selected from the group consisting of DNA nucleotides, RNA nucleotides, phosphorothioate-modified nucleotides, 2-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, DNA analogs with L-ribose (L-DNA), Xeno nucleic acid (XNA) analogues, or other nucleic acid analogues capable of base-pair formation, or artificial nucleic acid analogues with altered backbones, or any combination or mixture thereof.

For any of the any of the haplomer polynucleotides described herein, the complementarity with another nucleic acid molecule can be 100%. In some embodiments, one particular nucleic acid molecule can be substantially complementary to another nucleic acid molecule. As used herein, the phrase “substantially complementary” means from 1 to 10 mismatched base positions, from 1 to 9 mismatched base positions, from 1 to 8 mismatched base positions, from 1 to 7 mismatched base positions, from 1 to 6 mismatched base positions, from 1 to 5 mismatched base positions, from 1 to 4 mismatched base positions, from 1 to 3 mismatched base positions, and 1 or 2 mismatched base positions. In some embodiments, it is desirable to avoid reducing the Tₘ of the anti-target loop portion:target nucleic acid molecule by more than 10% via mismatched base positions. The bottle haplomer stem is designed with respect to a second haplomer, and its structure is deliberately arranged to be somewhat more stable than the formation of the second haplomer duplex.

In some embodiments, the portion of the bottle haplomer that is not linked to a protein fragment can have additional nucleotide bases that overhang and do not form a part of the stem structure. In some embodiments, the end of the second haplomer that is not linked to a protein fragment can have additional nucleotide bases that overhang and do not form a complementary part of the structure with the stem portion of the bottle haplomer. In addition, in some embodiments, the portion of the stem that is linked to the protein fragment can also have nucleotide bases that are not base paired with the first stem portion. Such an extension of the stem with a non-hybridized “arm” places the two protein fragments at a greater spatial distance, thus, tending to reduce their mutual reactivity. So, for a few nucleotide bases (less than 10 or less than 5), enforced reactivity is still likely to occur, but will tend to diminish as the non-base paired segment grows in length.

In some embodiments, added nucleotide bases can be of indefinite length, as long as they did not: 1) have significant homologies with any of the other regions of the locked TAPER oligonucleotides, and thus tend to cross-hybridize and interfere; or 2) interfere non-specifically with any other features of the system. For example, a long appended sequence might reduce transformation efficiencies of locked TAPER oligonucleotides used in a therapeutic context. Also, appended sequences should be designed to avoid spurious hybridizations with other cellular transcripts. Appended non-homologous sequences of 20-30 nucleotide bases are suitable. The appended nucleic acid sequences may contain primer sequences commonly used in the art. Such examples may include, but are not limited to, M13, T3, T7, SP6, VF2, VR, modified versions thereof, complementary sequences thereof, and reverse sequences thereof. In addition, custom primer sequences are also included. Such primer sequences can be used, for example, the possible application of chemically-ligated oligonucleotides spatially elicited (CLOSE) to the locked TAPER strategy, (see, PCT Publication WO 2016/89958; which is incorporated herein by reference in its entirety).

Any of the haplomers and bottle haplomers described herein, or any portion thereof, can further comprise a linker between any one or more of the first stem portion and the anti-target loop portion, between the anti-target loop portion and the second stem portion, between the second stem portion and the protein fragment, between the first stem portion and the ligand, or between the second haplomer and its protein fragment. In some embodiments, the linker is selected from the group consisting of an alkyl group, an alkenyl group, an amide, an ester, a thioester, a ketone, an ether, a thioether, a disulfide, an ethylene glycol, a cycloalkyl group, a benzyl group, a heterocyclic group, a maleimidyl group, a hydrazone, a urethane, azoles, an imine, a haloalkyl, nitrilotriacetic acid, nickel, cobalt, copper, and a carbamate, or any combination thereof.

In some embodiments, the bottle haplomer comprises the nucleotide sequence 5′-ACT CGAGACGTCTCCTTGTCTITGCTITITCAGGACACAGTGGCGAGACGTCTCGA GT-3′ (SEQ ID NO:28) or 5′-ACTCGAGACGTCTCCTTCCTGCCCCTCCTCCTGCTCCGA GACGTCTCGAGT-3′ (SEQ ID NO:29).

In some embodiments, the second haplomer comprises the nucleotide sequence 5′-AG CTCTCGAGT-3′ (SEQ ID NO:30), or 5′-GACGTCTCGAGT-3′ (SEQ ID NO:31).

In some embodiments, the polynucleotide of the bottle haplomer comprises the nucleotide sequence of 5′-ACTCGAGACGTCTCCT7GTCITrGCTTITCTTCAGGACAC AGTGGCGAGACGTCTCGAGT-3′ (SEQ ID NO:32), and the polynucleotide of the second haplomer comprises the nucleotide sequence of 5′-AGCTCTCGAGT-3′ (SEQ ID NO:30); or the polynucleotide of the bottle haplomer comprises the nucleotide sequence of 5′-ACTCGAGACG TCTCCTTCCTGCCCCTCCTCCTGCTCCGAGACGTCTCGAGT-3′ (SEQ ID NO:29), and the polynucleotide of the second haplomer comprises the nucleotide sequence 5′-GACGTCTCGA GT-3′ (SEQ ID NO:31).

The target nucleic acid molecules that serve as templates in the embodiments described herein can be comprised of any desired nucleic acid sequence capable of hybridizing with the polynucleotides of the haplomers or the anti-target loop portion of a bottle haplomer. Any single-stranded nucleic acid molecule with an accessible sequence is potentially targetable. These include, but are not limited to, cellular RNAs, mRNA, genomic or organellar DNA, episomal or plasmid DNA, viral DNA or RNA, miRNA, rRNA, snRNA, tRNA, short and long non-coding RNAs, and any artificial sequences used for templating purposes, or any other biological or artificial nucleic acid sequence. Artificial sequences include, but are not limited to, aptamers and macromolecular-nucleic acid conjugates. Aptamer templates are also included, where these are designed to convert a non-nucleic acid cellular product into a targetable sequence for any form of TAPER, including locked TAPER. In some embodiments, the target nucleic acid molecule hybridization site is kept as short as possible while: 1) maintaining specificity within a complex transcriptome or other complex targets; and 2) maintaining the locked TAPER design guidelines described herein.

Any cell, virus, tissues, spatial regions, lysate, or other subcomponent of a sample that contains a nucleic acid molecule can provide the target nucleic acid molecule. Target compartments that contain the target nucleic acid molecule can include, but are not limited to, pathogenic cells, cancer cells, viruses, host cells infected by a virus or other pathogen, or cells of the immune system that are contributing to autoimmunity such as cells of the adaptive or innate immune systems, transplant rejection, or an allergic response. In some embodiments, a target nucleic acid molecule can be present in a virus or cell infected by a virus, but absent in healthy cells. Examples of virus include, but are not limited to, DNA viruses, RNA viruses, or reverse transcribing viruses. In some embodiments, a target nucleic acid molecule can be present in a tumor or cancerous cell, but absent in healthy cells. Examples of cancers include, but are not limited to, those caused by oncoviruses, such as the human papilloma viruses, Epstein-Barr virus, hepatitis B virus, hepatitis C virus, human T-lymphotropic viruses, Merkel cell polyoma virus, and Kaposi's sarcoma-associated herpesvirus. In some embodiments, a target nucleic acid molecule can be present in an infectious agent or microbe, or a cell infected by an infectious agent or microbe but is absent in healthy cells. Examples of infectious agents or microbes include, but are not limited to, viruses, bacteria, fungi, protists, prions, or eukaryotic parasites.

The target nucleic acid molecule can also be a fragment, portion or part of a gene, such as an oncogene, a mutant gene, an oncoviral gene, a viral nucleic acid sequence, a microbial nucleic acid sequence, a differentially expressed gene, and a nucleic acid gene product thereof. In some embodiments, the target nucleic acid molecule is a cellular nucleic acid molecule, a tumor-specific nucleic acid molecule, an aberrant immune pathway nucleic acid molecule, or the polynucleotide of a surface target compound.

Examples of cancer-specific target nucleic acids include, but are not limited to, mutant oncogenes, such as mutated ras, HRAS, KRAS, NRAS, BRAF, EGFR, FLT1, FLT4, KDR, PDGFRA, PDGFRB, ABL1, PDGFB, MYC, CCND1, CDK2, CDK4, or SRC genes; mutant tumor suppressor genes, such as TP53, TP63, TP73, MDM1, MDM2, ATM, RB1, RBL1, RBL2, PTEN, APC, DCC, WT1, IRF1, CDK2AP1, CDKN1A, CDKN1B, CDKN2A, TRIM3, BRCA1, or BRCA2 genes; and genes expressed in cancer cells, where the gene may not be mutated or genetically altered, but is not expressed in healthy cells of a sample at the time of administration, such as carcinoembryonic antigen.

In some embodiments, the target nucleic acid molecule can be present in a differential amounts or concentrations in the target compartments as compared to the non-target compartments. Examples include, but are not limited to, genes expressed at a different level in cancer cells than in healthy cells, such as myc, telomerase, HER2, or cyclin-dependent kinases. In some embodiments, the target nucleic acid molecule can be a gene that is at least 1.5×-fold differentially expressed in the target versus the non-target compartments. Some examples of these include, but are not limited to, genes related to mediating Type I allergic responses, for which target RNA molecules contain immunoglobulin epsilon heavy chain sequences; genes expressed in T cell subsets, such as specific T cell receptors (TCRs) which recognize self-antigens in the context of particular major histocompatibility (MHC) proteins like proinsulin-derived peptide and clonally-specific mRNAs containing α or β variable-region sequences, derived from diabetogenic CD8+ T cells; and cytokines whose production may have adverse outcomes through exacerbation of inflammatory responses including, but not limited to, TNF-alpha, TNF-beta, IL-1, IL-2, IL-4, IL-6, IL-8, IL-10, IL-12, IL-15, IL-17, IL-18, IL-21, IL-22. IL-27. IL-31, IFN-gamma, OSM, and LIF.

In some embodiments, a target nucleic acid molecule is present in target compartments and an acceptable subgroup of non-target compartments, but not in a different or distinct subgroup of non-target compartments. Examples include, but are not limited to, genes expressed in cancer cells and limited to classes of healthy cells, such as cancer-testis antigens, survivin, prostate-specific antigen, carcinoembryonic antigen (CEA), alpha-fetoprotein and other onco-fetal proteins. Also, many tissues and organs are not essential to otherwise healthy life in the face of serious disease. For example, melanocyte antigens, such as Melan-A/MART-1 and gp100 are expressed on many malignant melanomas as well as normal melanocytes, and therapies that target these antigens can destroy both tumors and normal melanocytes, resulting in vitiligo, but major tumor reduction. Likewise, the reproductive organs may be surgically removed, such as testis, ovary and uterus, as well as associated organs such as breast and prostate may be targeted when tumors of these tissues arise, and destruction of normal tissues within these organs may be a tolerable consequence of therapy. Furthermore, some cells that produce hormones, such as thyroxine and insulin can be replaced with the relevant protein, allowing potential targeting of normal cells that may exist in the presence of tumors of these origins.

In some embodiments, the target nucleic acid molecule for a particular haplomer is the polynucleotide of the corresponding haplomer, such that Architecture 1 is produced.

Target nucleic acid molecules can also include novel sequences, not previously identified. In some embodiments, a sample or samples can be evaluated by sequence analysis, such as next-generation sequencing, whole-transcriptome (RNA-seq) or whole-genome sequencing, microarray profiling, serial analysis of gene expression (SAGE), to determine the genetic makeup of the sample. Target nucleic acid molecules can be identified as those present in target compartments, but not present in non-target compartments, or present in differential amounts or concentrations in target compartments as compared to non-target compartments. Sequences identified by these methods can then serve as target nucleic acid molecules.

In some embodiments, the polynucleotides of the haplomers and the protein fragments may further comprise a bio-orthogonal reactive moiety to assist their linkage. A bio-orthogonal moiety includes those groups that can undergo “click” reactions between azides and alkynes, traceless or non-traceless Staudinger reactions between azides and phosphines, and native chemical ligation reactions between thioesters and thiols. Additionally, the bio-orthogonal moiety can be any of an azide, an alkyne, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, a quadricyclane, and derivatives thereof. Bio-orthogonal moieties of members of a set of corresponding haplomers are selected such that they will react with each other.

Multiple bio-orthogonal moieties can be used with the methods and compositions disclosed herein, some non-limiting examples include:

### Azide-Alkyne “Click Chemistry”

Click chemistry is highly selective as neither azides nor alkynes react with common biomolecules under typical conditions. Azides of the form R—N₃ and terminal alkynes of the form R—C≡CH or internal alkynes of the form R—C≡C—R react readily with each other to produce Huisgen cycloaddition products in the form of 1,2,3-triazoles.

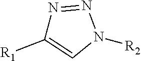

Azide-based haplomers have the substructure: R—N₃, where R is a chemical linker, nucleic acid recognition moiety (e.g. a portion of an oligonucleotide that is complementary to another portion of a nucleic acid molecule), or ligand. Azides and azide derivatives may be readily prepared from commercially available reagents.

Azides can also be introduced to a protein fragment or polynucleotide during synthesis of the protein fragment or polynucleotide. In some embodiments, an azide group is introduced into a protein fragment or polynucleotide by incorporation of a commercially available azide-derivatized standard amino acid or amino acid analogue during synthesis of the protein fragment or polynucleotide using standard peptide synthesis methods. Amino acids may be derivatized with an azide replacing the α-amino group, affording a structure of the form:

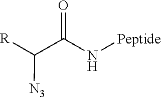

where R is a side chain of a standard amino acid or non-standard amino acid analogue.

Commercially available products can introduce azide functionality as amino acid side chains, resulting in a structure of the form:

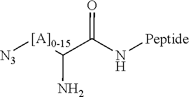

where A is any atom and its substituents in a side chain of a standard amino acid or non-standard amino acid analogue.

An azide may also be introduced into a protein fragment or polynucleotide after synthesis by conversion of an amine group on the protein fragment or polynucleotide to an azide by diazotransfer methods. Bioconjugate chemistry can also be used to join commercially available derivatized azides to chemical linkers, nucleic acid recognition moieties, or protein fragment or polynucleotide that contain suitable reactive groups.

Standard alkynes can be incorporated into a haplomer by methods similar to azide incorporation. Alkyne-functionalized nucleotide analogues are commercially available, allowing alkyne groups to be directly incorporated at the time of nucleic acid recognition moiety synthesis. Similarly, alkyne-deriviatized amino acid analogues may be incorporated into a protein fragment or polynucleotide by standard peptide synthesis methods. Additionally, diverse functionalized alkynes compatible with bioconjugate chemistry approaches may be used to facilitate the incorporation of alkynes to other moieties through suitable functional or side groups.

### Azide-Activated Alkyne “Click Chemistry”

Standard azide-alkyne chemistry reactions typically require a catalyst, such as copper(I). Since copper(I) at catalytic concentrations is toxic to many biological systems, standard azide-alkyne chemistry reactions have limited uses in living cells. Copper-free click chemistry systems based on activated alkynes circumvent toxic catalysts.

Activated alkynes often take the form of cyclooctynes, where incorporation into the cyclooctyl group introduces ring strain to the alkyne.

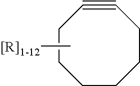

Heteroatoms or substituents may be introduced at various locations in the cyclooctyl ring, which may alter the reactivity of the alkyne or afford other alternative chemical properties in the compound. Various locations on the ring may also serve as attachment points for linking the cyclooctyne to a nucleic acid templated assembly moiety or linker. These locations on the ring or its substituents may optionally be further derivatized with accessory groups.

Multiple cyclooctynes are commercially available, including several derivatized versions suitable for use with standard bioconjugation chemistry protocols. Commercially available cyclooctyne derivatized nucleotides can aid in facilitating convenient incorporation of the protein fragment or polynucleotide during synthesis.

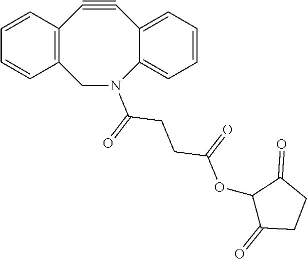 [SMILES: O=C(CCC(=O)N1Cc2ccccc2C#Cc2ccccc21)OC1C(=O)CCC1=O]

### Azide-Phosphine Staudinger Chemistry

The Staudinger reduction, based on the rapid reaction between an azide and a phosphine or phosphite with loss of N₂, also represents a bio-orthogonal reaction. The Staudinger ligation, in which covalent links are formed between the reactants in a Staudinger reaction, is suited for use in nucleic acid templated assembly. Both non-traceless and traceless forms of the Staudinger ligation allow for a diversity of options in the chemical structure of products formed in these reactions.

### Non-Traceless Staudinger Ligation

The standard Staudinger ligation is a non-traceless reaction between an azide and a phenyl-substituted phosphine such as triphenylphosphine, where an electrophilic trap substituent on the phosphine, such as a methyl ester, rearranges with the aza-ylide intermediate of the reaction to produce a ligation product linked by a phosphine oxide.

Phenyl-substituted phosphines carrying electrophilic traps can also be readily synthesized. Derivatized versions are available commercially and suitable for incorporation into haplomers:

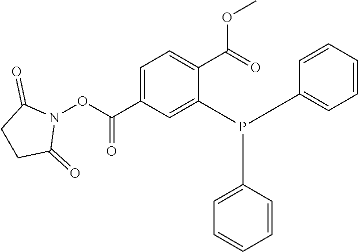 [SMILES: COC(=O)c1ccc(C(=O)ON2C(=O)CCC2=O)cc1P(c1ccccc1)c1ccccc1]

### Traceless Staudinger Ligation

In some embodiments, phosphines capable of traceless Staudinger ligations may be utilized as bio-orthogonal moieties for polynucleotides and protein fragments. In a traceless reaction, the phosphine serves as a leaving group during rearrangement of the aza-ylide intermediate, creating a ligation typically in the form of a native amide bond. Compounds capable of traceless Staudinger ligation generally take the form of a thioester derivatized phosphine or an ester derivatized phosphine:

An exemplary ester-derivatized phosphine for traceless Staudinger ligation is:

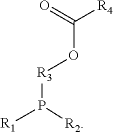

An exemplary thioester-derivatized phosphine for traceless Staudinger ligations is:

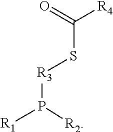

Chemical linkers or accessory groups may optionally be appended as substituents to the R groups in the above structures, providing attachment points for polynucleotides and protein fragments or for the introduction of additional functionality to the reactant.

### Traceless Phosphinophenol Staudinger Ligation

Compared to the non-traceless Staudinger phenylphosphine compounds, the orientation of the electrophilic trap ester on a traceless phosphinophenol is reversed relative to the phenyl group. This enables traceless Staudinger ligations to occur in reactions with azides, generating a native amide bond in the product without inclusion of the phosphine oxide.

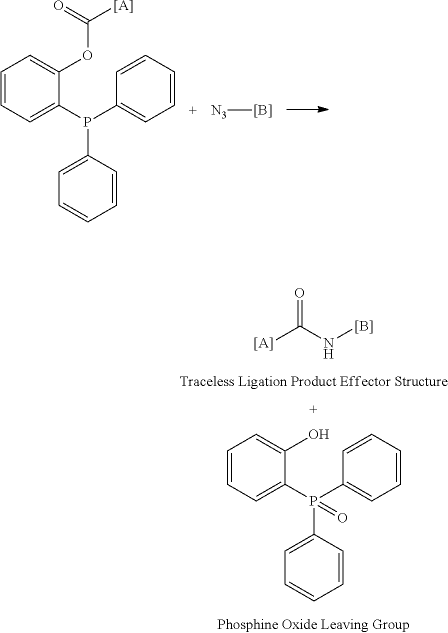

The traceless Staudinger ligation may be performed in aqueous media without organic co-solvents if suitable hydrophilic groups, such as tertiary amines, are appended to the phenylphosphine. Weisbrod and Marx describes preparation of water-soluble phosphinophenol, which may be loaded with a desired ligand containing a carboxylic acid (such as the C-terminus of a peptide) via the mild Steglich esterification using a carbodiimide such as dicyclohexylcarbodiimide (DCC) or N,N′-diisopropylcarbodiimide (DIC) and an ester-activating agent such as 1-hydroxybenzotriazole (HOBT). This approach facilitates synthesis of haplomers of the form:

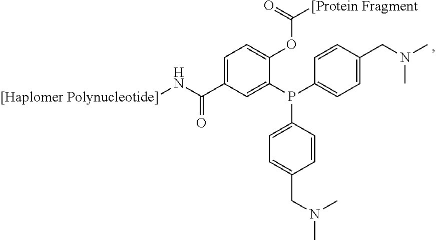

### (Synlett, 2010, 5, 787-789).

Water-soluble phosphinophenol-based traceless haplomer structure.

### Traceless Phosphinomethanethiol Staudinger Ligation

Phosphinomethanethiols represent an alternative to phosphinophenols for mediating traceless Staudinger ligation reactions. In general, phosphinomethanethiols possess favorable reaction kinetics compared with phosphinophenols in mediating traceless Staudinger reaction. U.S. Patent Application Publication 2010/0048866 and Tam et al., J. Am. Chem. Soc., 2007, 129, 11421-30 describe preparation of water-soluble phosphinomethanethiols of the form:

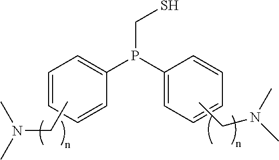

These compounds may be loaded with a peptide or other payload, in the form of an activated ester, to form a thioester suitable for use as a traceless bio-orthogonal reactive group.

### Native Chemical Ligation

Native chemical ligation is a bio-orthogonal approach based on the reaction between a thioester and a compound bearing a thiol and an amine. The classic native chemical ligation is between a peptide bearing a C-terminal thioester and another bearing an N-terminal cysteine, as seen below:

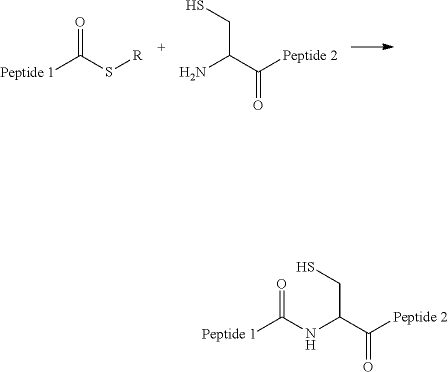

Native chemical ligation may be utilized to mediate traceless reactions producing a peptide or peptidomimetic containing an internal cysteine residue, or other thiol-containing residue if non-standard amino acids are utilized.

N-terminal cysteines may be incorporated by standard amino acid synthesis methods. Terminal thioesters may be generated by several methods known in the art, including condensation of activated esters with thiols using agents such as dicyclohexylcarbodiimide (DCC), or introduction during peptide synthesis via the use of “Safety-Catch” support resins.

### Other Selectively Reactive Moieties

Any suitable bio-orthogonal reaction chemistry may be utilized for synthesis of haplomer-protein fragment complexes, as long as it efficiently mediates a reaction in a highly selective manner in complex biologic environments. A recently developed non-limiting example of an alternative bio-orthogonal chemistry that may be suitable is reaction between tetrazine and various alkenes such as norbornene and trans-cyclooctene, which efficiently mediates bio-orthogonal reactions in aqueous media.

Chemical linkers or accessory groups may optionally be appended as substituents to the above structures, providing attachment points for polynucleotides or protein fragments, or for the introduction of additional functionality to the reactant.

The configurations involving the protein fragments depicted in the Examples and Figures could be reversed. In other words, the protein fragment could be linked to the 3′ end of the bottle haplomer, as long as the second haplomer accordingly had its protein fragment linked to its 5′ end. The Examples provided below have the bottle haplomer with a 5′-linked protein fragment and the second haplomer with a 3′-linked protein fragment. Likewise, in this system, the bio-orthogonal moieties can be switched around. For example, instead of using the bottle haplomer with a 5′-hexynyl and the second haplomer with a 3′-azide, the bottle haplomer could bear the azide, and the second haplomer could bear the hexynyl group.

In some embodiments, the bio-orthogonal moiety is chosen from an azide, an alkyne, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, or a quadricyclane, or any derivative thereof. In some embodiments, the bio-orthogonal moiety of the first haplomer is hexynyl and the bio-orthogonal moiety of the second haplomer is azide. In some embodiments, the bio-orthogonal moiety of the first haplomer is azide and the bio-orthogonal moiety of the second haplomer is hexynyl.

In some embodiments, the protein of interest produced by the templated assembly may trigger activity by acting within a target compartment (for example, within a cell), at the surface of a target compartment (for example, at the cell surface), in the vicinity of the target compartment (for example, when the effector structure is actively exported from the cell, leaks from the cell, or released upon cell death), or diffuse or be carried to a distant region of the sample to trigger a response. In some embodiments, the protein of interest can be targeted to their active sites by incorporation of targeting groups in the templated assembly product. Examples of targeting groups include, but are not limited to, endoplasmic reticulum transport signals, golgi apparatus transport signals, nuclear transport signals, mitochondrial transport signals, ubiquitination motifs, other proteosome targeting motifs, and glycosylphosphatidylinositol anchor motifs. Targeting groups may be introduced by their incorporation into a haplomer moiety, chemical linker, or accessory group during synthesis, or may be generated during the ligation reaction.

In some embodiments, the protein of interest can be presented on the surface of a target compartment. In some embodiments, the protein of interest can be presented on the surface of a cell as a ligand bound to a major histocompatibility complex molecule.

In some embodiments, the protein of interest can be an endogenous peptide, and their analogue, or a completely synthetic structure which is a target for agents such as antibodies. Because the availability of target nucleic acid molecules can limit production of active proteins of interest, it may be desirable to have proteins of interest that exert activity when present at low levels.

In some embodiments, the N-terminal protein fragment and C-terminal protein fragment are both derived from a reporter protein, a transcription factor, a signal transduction pathway factor, a gene editing protein, a single-chain immunoglobulin variable region (scFv) protein, a toxic protein, or an enzyme.

In some embodiments, the enzyme is a 8-lactamase, a choramphenicol acetyl transferase, an aminoglycoside-3′-phosphotransferase, 8-galactosidase, a dihydrofolate reductase, a restriction enzyme, a DNase, or an RNase.

In some embodiments, the reporter protein is a fluorescent protein, a luciferase, a choramphenicol acetyl transferase, a 8-galactosidase, or a 8-glucuronidase.

In some embodiments, the fluorescent protein is GFP, YFP, mCherry, dsRed, VENUS, or CFP, a blue fluorescent protein, or any analog thereof. In some embodiments, the fluorescent protein is superfolder GFP. In some embodiments, the N-terminal fragment of the superfolder GFP comprises the amino acid sequence of MSKGEELFTGVVPILVELDGDVNGHKFSVRGE GEGDATNGKLTLKFICTTGKLPVPWPTLVTTLTYGVQCFSRYPDHMKRHDFFKSAMPE GYVQERTISFKDDGTYKTRAEVKFEGDTLVNRIELKGIDFKEDGNILGHKLEYNFNSHN VYITADKQ (SEQ ID NO:33). In some embodiments, the C-terminal fragment of the superfolder GFP comprises the amino acid sequence of KNGIKANFKIRHNVEDGSVQLADH YQQNTPIGDGPVLLPDNHYLSTQSVLSKDPNEKRDHMVLLEFVTAAGITHGMDELYK (SEQ ID NO:34). In some embodiments, the fragment of superfolder GFP (sfGFP) comprises MRKGEELFTGVVPILVELDGDVNGHKFSVRGEGEGDATNGKLTLKFICTTGKLPVPWPT LVTTLTYGVQCFARYPDHMKQHDFFKSAMPEGYVQERTISFKDDGTYKTRAEVKFEGD TLVNRIELKGIDFKEDGNILGHKLEYNFNSHNVYITADKQ (SEQ ID NO:35) or KNGIKAN FKIRHNVEDGSVQLADHYQQNTPIGDGPVLLPDNHYLSTQSVLSKDPNEKRDHMVLLEF VTAAGITHGMDELYK (SEQ ID NO:34), wherein one fragment interacts with the other fragment.

In some embodiments, the luciferase is firefly luciferase, Renilla luciferase, or Gaussia princeps luciferase. In some embodiments, the luciferase is Renilla luciferase. In some embodiments, the N-terminal fragment of the Renilla luciferase comprises the amino acid sequence of MASKVYDPEQRKRMITGPQWWARCKQMNVLDSFINYYDSEKHAENAVIF LHGNAASSYLWRHVVPHIEPVARCIIPDLIGMGKSGKSGNGSYRLLDHYKYLTAWFELL NLPKKIIFVGHDWGACLAFHYSYEHQDKIKAIVHAESVVDVIESWDEWPDIEEDIALIKS EEGEKMVLENNFFVETMLPSKIMRKLEPEEFAAYLEPFKEKGEVRRPTLSWPREIPLVKG GY (SEQ ID NO:36). In some embodiments, the C-terminal fragment of the Renilla luciferase comprises the amino acid sequence of KPDVVQIVRNYNAYLRASDDLPKMFIESDPGFFSNA IVEGAKKFPNTEFVKVKGLHFSQEDAPDEMGKYIKSFVERVLKNEQZ (SEQ ID NO:37). In some embodiments, the fragment of Renilla luciferase comprises MASKVYDPEQRKRMITG PQWWARCKQMNVLDSFINYYDSEKHAENAVIFLHGNAASSYLWRHVVPHIEPVARCIIP DLIGMGKSGKSGNGSYRLLDHYKYLTAWFELLNLPKKIIFVGHDWGACLAFHYSYEHQ DKIKAIVHAESVVDVIESWDEWPDIEEDIALIKSEEGEKMVLENNFFVETMLPSKIMRKL EPEEFAAYLEPFKEKGEVRRPTLSWPREIPLVKGG (SEQ ID NO:38) or KPDVVQIVRNYN AYLRASDDLPKMFIESDPGFFSNAIVEGAKKFPNTEFVKVKGLHFSQEDAPDEMGKYIKS FVERVLKNEQ (SEQ ID NO:39), wherein one fragment interacts with the other fragment. In some embodiments, the luciferase is Gaussia princeps luciferase. In some embodiments, the N-terminal fragment of the Gaussia princeps luciferase comprises the amino acid sequence of MKPTENNEDFNIVAVASNFATTDLDADRGKLPGKKLPLEVLKEMEANARKAGCTRGCL ICLSHIKCTPKMKKFIPGRCHTYEGDKESAQGGIG (SEQ ID NO:42). In some embodiments, the C-terminal fragment of the Gaussia princeps luciferase comprises the amino acid sequence of EAIVDIPEIPGFKDLEPMEQFIAQVDLCVDCTTGCLKGLANVQCSDLLKKWLPQRCAT FASKIQGQVDKIKGAGGD (SEQ ID NO:43).

In some embodiments, killing or growth inhibition of target cells can be induced by direct interaction with cytotoxic, microbicidal, or virucidal effector structures. Numerous toxic molecules known in the art can be produced. In some embodiments, the protein of interest is a toxic peptide or toxic protein. Examples of toxic peptides include, but are not limited to, bee melittin, conotoxins, cathelicidins, defensins, protegrins, and NK-lysin. Examples of toxic proteins include, but are not limited to, ricin A chain, Aspf1, α-sarcin, mitogillin, hirsutellin A, diphtheria toxin, botulinum A toxin, and cholera toxin. In some embodiments, the toxic protein is a ribotoxin that cleaves the large 28S ribosomal RNA.

In some embodiments, killing or growth inhibition of target cells can be induced by pro-apoptotic proteins of interest. For example, proteins of interest include pro-apoptotic peptides, including but not limited to, prion protein fragment 106-126 (PrP 106-126), Bax-derived minimum poropeptides associated with the caspase cascade including Bax 106-134, and pro-apoptotic peptide (KLAKLAK)₂.

In some embodiments, the protein of interest can be thrombogenic, in that it induces activation of various components of the clotting cascade of proteins, or activation of proteins, or activation and/or aggregation of platelets, or endothelial damage that can lead to a biologically active process in which a region containing pathogenic cells can be selectively thrombosed to limit the blood supply to a tumor or other pathogenic cell. These types of proteins of interest can also induce clotting, or prevent clotting, or prevent platelet activation and aggregation in and around targeted pathogenic cells.

In some embodiments, proteins of interest can mediate killing or growth inhibition of target cells or viruses by activating molecules, pathways, or cells associated with the immune system. Proteins of interest may engage the innate immune system, the adaptive immune system, and/or both.

In some embodiments, proteins of interest can mediate killing or growth inhibition of cells or viruses by stimulation of the innate immune system. In some embodiments, proteins of interest include pathogen-associated molecular patterns (PAMPs), damage-associated molecular patterns (DAMPs), and synthetic analogues thereof.

In some embodiments, the innate immune system can be engaged by proteins of interest that activate the complement system. A non-limiting example of a complement activating effector structures can be the C3a fragment of complement protein C3.

In some embodiments, proteins of interest can be natural or synthetic ligands of Toll-Like Receptors (TLR). Examples of such proteins of interest include peptide fragments of heat shock proteins (hsp) known to be TLR agonists.

In some embodiments, traceless bio-orthogonal chemistry may be used to produce the muramyl dipeptide agonist of the NOD2 receptor to activate an inflammatory response.

In some embodiments, proteins of interest can mediate killing or growth inhibition of cells or viruses by activating molecules or cells of the adaptive immune system. Unique to the adaptive immune system, molecules or cells can be engineered to recognize an extraordinary variety of structures, thus removing the constraint that the proteins of interest must be intrinsically active or bind to an endogenous protein.

In some embodiments, proteins of interest can be a ligand for an antibody or antibody fragment (including but not limited to Fab, Fv, and scFv). Traceless bio-orthogonal approaches can be used to produce a peptide or other epitope that is bound by an existing antibody, or an antibody can be developed to recognize proteins of interest created.

In some embodiments, the protein of interest is a fragment of: a cytotoxic protein, a microbicidal protein, a virucidal protein, a pro-apoptotic protein, a thrombogenic protein, a complement activating protein, a Toll-Like Receptor protein, a NOD2 receptor agonist protein, or an antibody or fragment thereof, wherein the first fragment and the second fragment interact to produce a functional protein.

In some embodiments, the cytotoxic protein is a bee melittin, a conotoxin, a cathelicidin, a defensin, a protegrin, or NK-lysin. In some embodiments, the pro-apoptotic protein is prion protein, a Bax-derived minimum poropeptide associated with the caspase cascade, or a pro-apoptotic peptide (KLAKLAK)₂ (SEQ ID NO:40). In some embodiments, the innate immune system stimulation protein is a pathogen-associated molecular pattern (PAMP) or a damage-associated molecular pattern (DAMP). In some embodiments, the complement activating protein is a C3a fragment of complement protein C3. In some embodiments, the Toll-Like Receptor (TLR) protein is a heat shock protein (hsp). In some embodiments, the NOD2 receptor agonist protein is muramyl dipeptide agonist. In some embodiments, the antibody fragment is an Fab, Fv, or scFv.

In some embodiments, the protein of interest is a fragment of: murine dihydrofolate reductase (DHFR), S. cerevisiae ubiquitin, β-lactamase, or Herpes simplex virus type 1 thymidine kinase, wherein one fragment of the protein of interest dimerizes or folds together with the other fragment of the protein of interest.

In some embodiments, the fragment of murine dihydrofolate reductase (DHFR) comprises amino acids 1-105 or 106-186 thereof, wherein one fragment interacts with the other fragment.

In some embodiments, the fragment of S. cerevisiae ubiquitin comprises amino acids 1-34 (MQIFVKTLTGKTITLEVESSDTIDNVKSKIQDKE; SEQ ID NO:55) or 35-76 (GIPPD QQRLIFAGKQLEDGRTLSDYNIQKESTLHLVLRLRGG; SEQ ID NO:56) thereof, wherein one fragment interacts with the other fragment.

In some embodiments, the fragment of β-lactamase comprises amino acids 25-197 or 198-286 thereof, wherein one fragment interacts with the other fragment.

In some embodiments, the fragment of Herpes simplex virus type 1 thymidine kinase comprises amino acids 1-265 or 266-376 thereof, wherein one fragment interacts with the other fragment.

In some embodiments, there may be no pre-existing information regarding where a protein of interest may be divided for general split-protein analyses, including SP-TAPER. In such cases, inspection of the three-dimensional crystal structure of the protein may provide a number of candidate targets within surface loops and turns, away from regions directly concerned with the protein's function. Fragments arising from cleavage at a predicted target site may be screened by separate expression as fusion proteins with, for example, suitable mutually interactive leucine zippers, where protein activity is restored upon mixing of fusion proteins if the split protein targeting is successful. More rapid assays for empirically flagging suitable cleavage sites are available, including solubility assays (see, Chen et al., Protein Science, 2009, 18, 399-409), or the preferred circular permutation assay (see, Massoud et al., Nature Medicine, 2010, 16, 921-926). These assays are applicable even in the absence of structural information, but can be guided and made more efficient by structural knowledge where available. For the circular permutation assay, a tandem in-frame continuous dimer of the coding sequence of interest is initially generated, with a serine-glycine linker (such as [SGGGG]₃; SEQ ID NO:57) positioned between the two copies. Circularly permuted coding sequence blocks for expression are then generated from the dimer by amplification using suitable primers.

The present disclosure also provides compositions or kits comprising any one or more of the haplomers, bottle haplomers, and surface target compounds described herein.

In some embodiments, the compositions or kits comprise: a) a first haplomer, wherein the first haplomer comprises a polynucleotide linked to the C-terminus of an N-terminal protein fragment; and b) a second haplomer, wherein the second haplomer comprises a polynucleotide linked to the N-terminus of a C-terminal protein fragment; wherein: i) the polynucleotide of one of the first or second haplomers is linked at its 5′ terminus to the protein fragment, and the other of the first and second haplomers is linked at its 3′ terminus to the protein fragment; and ii) the N-terminal protein fragment and the C-terminal protein fragment are derived from a single protein; and wherein: i) the polynucleotide of the first haplomer is complementary to the polynucleotide of the second haplomer; or ii) the polynucleotide of the first haplomer is complementary to a target nucleic acid molecule, and the polynucleotide of the second haplomer is substantially complementary to the target nucleic acid molecule at a site in spatial proximity to the polynucleotide of the first haplomer; or iii) the polynucleotide of the first haplomer is substantially complementary to a portion of a target nucleic acid molecule 5′ adjacent to a stem-loop structure, and the polynucleotide of the second haplomer is substantially complementary to a portion of the target nucleic acid molecule 3′ adjacent to the stem-loop structure; or iv) the polynucleotide of the first haplomer is substantially complementary to a 5′ portion of a loop of a stem-loop structure of a target nucleic acid molecule, and the polynucleotide of the second haplomer is substantially complementary to a 3′ portion of the loop of the stem-loop structure of the target nucleic acid molecule.

In some embodiments, the compositions or kits comprise: a) a bottle haplomer comprising a polynucleotide comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein: i) the 5′ terminus of the polynucleotide comprises a —SH moiety; and ii) the Tₘ of the anti-target loop portion:target nucleic acid molecule is greater than the Tₘ of the first stem portion:second stem portion; b) an N-terminal protein fragment, wherein the C-terminus of the N-terminal protein fragment comprises a cysteine-SH moiety; and c) a bis-maleimide reagent.

In some embodiments, the compositions or kits comprise: a) a bottle haplomer comprising a polynucleotide comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and b) a second haplomer comprising a polynucleotide and a C-terminal protein fragment, wherein the 3′ terminus of the polynucleotide is linked to the N-terminus of the C-terminal protein fragment, wherein the N-terminus comprises a cysteine; wherein: i) the polynucleotide of the second haplomer is substantially complementary to the second 5′ stem portion of the polynucleotide of the bottle haplomer; ii) the Tₘ of the anti-target loop portion:target nucleic acid molecule is greater than the Tₘ of the first stem portion:second stem portion; and iii) the N-terminal protein fragment and the C-terminal protein fragment are derived from a single protein.

In some embodiments, the Tₘ of the first stem portion:second stem portion subtracted from the Tₘ of the anti-target loop portion:target nucleic acid molecule is from about 10° C. to about 40° C. In some embodiments, the Tₘ of the first stem portion:second stem portion subtracted from the Tₘ of the anti-target loop portion:target nucleic acid molecule is from about 10° C. to about 20° C. In some embodiments, the Tₘ of the first stem portion:second stem portion is from about 40° C. to about 50° C. In some embodiments, the Tₘ of the anti-target loop portion:target nucleic acid molecule is from about 60° C. to about 80° C. In some embodiments, the Tₘ of the duplex formed by the second haplomer and the first or second stem portion of the bottle haplomer subtracted from the Tₘ of the first stem portion:second stem portion is from about 0° C. to about 20° C. In some embodiments, the Tₘ of the duplex formed by the second haplomer and the first or second stem portion of the bottle haplomer subtracted from the Tₘ of the first stem portion:second stem portion is from about 5° C. to about 10° C. In some embodiments, the Tₘ of the duplex formed by the second haplomer and the first or second stem portion of the bottle haplomer is from about 30° C. to about 40° C.

In some embodiments, the first stem portion comprises from about 12 to about 18 nucleotide bases. In some embodiments, the anti-target loop portion comprises from about 18 to about 35 nucleotide bases. In some embodiments, the second stem portion comprises from about 12 to about 18 nucleotide bases.

In some embodiments, the compositions or kits further comprise a protein chaperone, a small-molecule chaperone, or a pharmacoperone. In some embodiments, the protein chaperone is a heat-shock protein. In some embodiments, the small-molecule chaperone is 4-phenyl butyrate, deoxycholic acid, ursodeoxycholic acid, taurourso-deoxycholic acid, lysophosphatidic acid, trehalose, mannitol, trimethylamine oxide, betaine, or dimethylsulfoxide.

The present disclosure also provides methods of cleaving an N-terminal protein fragment from an intein fusion partner in a fusion protein comprising: a) contacting the fusion protein with 2-mercaptoethane sulfonic acid; and b) contacting the fusion protein with a cysteine having a methyltetrazine group; thereby releasing the N-terminal protein fragment from the fusion protein. In some embodiments, the cysteine having a methyltetrazine group is

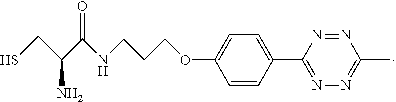 [SMILES: Cc1nnc(-c2ccc(OCCCNC(=O)[C@@H](N)CS)cc2)nn1]

In some embodiments, the method further comprises reacting the N-terminal protein fragment with a polynucleotide having a 5′ or 3′ trans-cyclooctene group.

The present disclosure also provides methods of using the any of the haplomers described herein for the directed assembly of a protein.

In some embodiments, the method comprises: a) contacting a cell with a first haplomer comprising a polynucleotide linked to the C-terminus of an N-terminal protein fragment; and

b) contacting the cell with a second haplomer comprising a polynucleotide linked to the N-terminus of a C-terminal protein fragment; wherein: i) the polynucleotide of one of the first or second haplomers is linked at its 5′ terminus to the protein fragment, and the other of the first and second haplomers is linked at its 3′ terminus to the protein fragment; ii) the N-terminal protein fragment and the C-terminal protein fragment are derived from a single protein; and iii) wherein: the polynucleotide of the first haplomer is substantially complementary to the polynucleotide of the second haplomer; or the polynucleotide of the first haplomer is substantially complementary to a target nucleic acid molecule, and the polynucleotide of the second haplomer is substantially complementary to the target nucleic acid molecule at a site in spatial proximity to the polynucleotide of the first haplomer; or the polynucleotide of the first haplomer is substantially complementary to a portion of a target nucleic acid molecule 5′ adjacent to a stem-loop structure, and the polynucleotide of the second haplomer is substantially complementary to a portion of the target nucleic acid molecule 3′ adjacent to the stem-loop structure; or the polynucleotide of the first haplomer is substantially complementary to a 5′ portion of a loop of a stem-loop structure of a target nucleic acid molecule, and the polynucleotide of the second haplomer is substantially complementary to a 3′ portion of the loop of the stem-loop structure of the target nucleic acid molecule; thereby resulting in the assembly of the protein from the N-terminal protein fragment and the C-terminal protein fragment.

In some embodiments, the polynucleotide of the first haplomer is substantially complementary to the polynucleotide of the second haplomer. In some embodiments, the polynucleotide of the first haplomer binds to the target nucleic acid molecule in spatial proximity to the binding of the polynucleotide of the second haplomer to the target nucleic acid molecule.

In some embodiments, the protein fragment of the first haplomer is linked to the 5′ terminus of the polynucleotide of the first haplomer, and the polynucleotide of the first haplomer is substantially complementary to a portion of the nucleic acid target 5′ adjacent to a stem-loop structure; and the protein fragment of the second haplomer is linked to the 3′ terminus of the polynucleotide of the second haplomer, and the polynucleotide of the second haplomer is substantially complementary to a portion of the nucleic acid target 3′ adjacent to the stem-loop structure.

In some embodiments, the protein fragment of the first haplomer is linked to the 3′ terminus of the polynucleotide of the first haplomer, and the polynucleotide of the first haplomer is substantially complementary to a 5′ portion of a loop structure of a stem-loop structure of the target nucleic acid molecule, wherein the 5′ portion of the loop structure is adjacent to the stem region of the stem-loop structure; and the protein fragment of the second haplomer is linked to the 5′ terminus of the polynucleotide of the second haplomer, and the polynucleotide of the second haplomer is substantially complementary to a 3′ portion of the loop structure of the stem-loop structure of the target nucleic acid molecule, wherein the 3′ portion of the loop structure is adjacent to the stem region of the stem-loop structure.

In some embodiments, the method comprises: a) contacting a target nucleic acid molecule with a bottle haplomer comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and b) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer; wherein: i) the N-terminal protein fragment and the C-terminal protein fragment are derived from a single protein; ii) the Tₘ of the anti-target loop portion:target nucleic acid molecule is greater than the Tₘ of the first stem portion:second stem portion; and iii) the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

In some embodiments, the method comprises: a) contacting a cell with a surface target compound comprising: i) a template polynucleotide; and ii) a peptide; wherein: i) the 5′ terminus of the polynucleotide is coupled to the N-terminus or C-terminus of the peptide, or the 3′ terminus of the polynucleotide is coupled to the N-terminus or C-terminus of the peptide: and ii) the peptide is a ligand for a cell-surface molecule; b) contacting the cell with a first haplomer comprising a polynucleotide linked to the C-terminus of an N-terminal protein fragment; and c) contacting the cell with a second haplomer comprising a polynucleotide linked to the N-terminus of a C-terminal protein fragment; wherein: i) the polynucleotide of one of the first or second haplomers is linked at its 5′ terminus to the protein fragment, and the other of the first and second haplomers is linked at its 3′ terminus to the protein fragment; ii) the N-terminal protein fragment and the C-terminal protein fragment are derived from a single protein; and iii) the polynucleotide of the first haplomer is substantially complementary to the template polynucleotide of the surface target compound, and the polynucleotide of the second haplomer is substantially complementary to the template polynucleotide of the surface target compound at a site in spatial proximity to the polynucleotide of the first haplomer; thereby resulting in the assembly of the protein from the N-terminal protein fragment and the C-terminal protein fragment.

In some embodiments, the method comprises: a) contacting a cell with a surface target compound comprising: i) a template polynucleotide; and ii) a peptide; wherein: i) the 5′ terminus of the polynucleotide is coupled to the N-terminus or C-terminus of the peptide, or the 3′ terminus of the polynucleotide is coupled to the N-terminus or C-terminus of the peptide; and ii) the peptide is a ligand for a cell-surface molecule; b) contacting a target nucleic acid molecule with a bottle haplomer comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to the template polynucleotide of the surface target compound; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and c) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer; wherein: i) the N-terminal protein fragment and the C-terminal protein fragment are derived from a single protein; ii) the Tₘ of the anti-target loop portion:target nucleic acid molecule is greater than the Tₘ of the first stem portion:second stem portion; and iii) the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

The present disclosure also provides methods of using any of the haplomers described herein to modulate a cell or cell target molecule. Administration of sets of corresponding haplomers to a mammal, or to a human, may vary according to the nature of the disease, disorder or condition sought to be treated. In some embodiments, the haplomers and bottle haplomers can be dispensed into a sample within a suitable vessel or chamber. In some embodiments, the sample may be dispensed into a vessel already containing the haplomers or bottle haplomers. In some embodiments, the haplomers and bottle haplomers can be used in vitro or in situ. In some embodiment, the human will be in need of such treatment.

In some embodiments, the haplomers and bottle haplomers can be administered for templated assembly in vivo. To facilitate such treatment, prepared haplomers and bottle haplomers may be administered in any suitable buffer or formulation, optionally incorporating a suitable delivery agent, and contacted with the mammal or human, or sample thereof for ex vivo methods. Concentrated forms of haplomers and bottle haplomers may be handled separate from its counterpart haplomers and bottle haplomers, as product-generating reactions may occur in the absence of target nucleic acid molecule template at high concentrations. Table 1 provides guidelines for maximum acceptable concentrations of gymnotic (no delivery agent) haplomers and bottle haplomers. If the haplomers and bottle haplomers are contacted at concentrations above these thresholds, non-templated background reactions may occur.

TABLE 1
Maximum concentrations for contact of haplomers, above
which non-templated reaction levels may occur

| | Maximum |
|---|---|
| Bioorthogonal Reactive Chemistry | Concentration |
| Azide-Alkyne | <50 μM |
| Azide-Phosphine | <50 μM |
| Native Chemical Ligation | <1 mM |

Threshold concentrations of other haplomers and bottle haplomers may be determined empirically utilizing the templated assembly diagnostic evaluation assay disclosed.

In some embodiments, the likelihood of non-templated reactions may be reduced by administering a set of corresponding haplomers and bottle haplomers such that one haplomer is administered first, then a time delay is observed before the corresponding haplomer is administered. This time delay may range from one minute to days, depending on the persistence of the haplomer in the system.

Certain delivery agents, such as transfection reagents such as cationic lipids, polyethyleneimine, dextran-based transfectants, or others known in the art, may cause condensation of the haplomers. Under these circumstances, haplomers may be prepared separate from the corresponding reactive haplomers and administered to the sample separately. Haplomers may also be administered gymnotically, dissolved in an appropriate buffer without addition of any additional delivery agent.

The haplomers and bottle haplomers may also be administered after formulation with a suitable delivery agent. A suitable delivery agent may enhance the stability, bioavailability, biodistribution, cell permeability, or other desirable pharmacologic property of the haplomers and bottle haplomers, or a combination of these properties. Delivery agents known in the art include, but are not limited to, polycationic transfection reagents, polyethyleneimine and its derivatives, DEAE-Dextran, other transfection reagents, salts, ions, buffers, solubilization agents, various viral vectors, liposomes, targeted liposomes, nanoparticles, carrier polymers, endosome disruptors, permeabilization agents, lipids, steroids, surfactants, dispersants, stabilizers, or any combination thereof.

Delivery of haplomers and bottle haplomers to target compartments may also be enhanced by covalent attachment of accessory groups to haplomers and bottle haplomers. Accessory groups that may enhance delivery may include compounds known to enhance the stability and biodistribution of compounds, such as polyethylene glycol (PEG); and enhance cell permeability of haplomers, including, but not limited to, cholesterol derivatives known in the art, endosome-disrupting agents known in the art, and cell-penetrating peptides, such as poly-cations such as poly-arginine or polylysine, peptides derived from the HIV tat protein, transportan, and peptides derived from the antennapedia protein (penetratin).

Administration of effector protein product-triggered agents, such as an antibody or other effector protein product-detecting molecule, or effector protein product-detecting cell, may also be included. The administration can be part of the templated assembly procedure. It may be administered before, during, or after administration of the haplomers and bottle haplomers, and by any method appropriate to the agent. In some embodiments, the effector protein product-triggered agent is administered prior to administration of the haplomers and bottle haplomers to facilitate triggering of activity by effector proteins as soon as they are formed and available for agent binding.

In some embodiments, multiple sets of corresponding haplomers and bottle haplomers may be administered in parallel. These sets of reactants may bind to multiple hybridization sites on a single target nucleic acid molecule, or bind to different target nucleic acid molecules, or a combination thereof. The different sets of haplomers and bottle haplomers may produce the same protein structure, thus increasing the level of activity generated by that protein structure by boosting its production, or the different sets of haplomers and bottle haplomers may produce different protein structures, thus producing multivalent activity in the sample, or a combination thereof.

Production of effector proteins by the methods described herein can yield activities, such as, inducing an immune response, programmed cell death, apoptosis, necrosis, lysis, growth inhibition, inhibition of viral infection, inhibition of viral replication, inhibition of oncogene expression, modification of gene expression, inhibition of microbial infection, and inhibition of microbe replication, as well as combinations of these biological activities.

In some embodiments, the composition administered can include two or more sets of corresponding haplomers and bottle haplomers that target two or more target nucleic acid molecules. Two or more target nucleic acid molecules may be found within the same gene transcript, or alternatively on distinct and separate transcripts. Two or more sets of corresponding haplomers and bottle haplomers recognizing distinct nucleic acid target molecules within the same cellular transcript may independently produce the same or different proteins.

The abundance of target nucleic acid molecules may also limit the amount of active protein produced by templated assembly. In some embodiments, there is an average of at least 5 copies of target nucleic acid molecules per target compartment. The dosage and concentration of the composition administered can take the availability of the target nucleic acid molecules into account.

In some embodiments, methods of delivering haplomers and bottle haplomers or a composition comprising one or more sets of the same to a pathogenic cell is disclosed. The methods can include administering a therapeutically effective amount of a set or multiple sets of corresponding haplomers and bottle haplomers compositions to the pathogenic cell. In some embodiments, the methods can also include detecting the presence or absence of the target nucleic acid molecule prior to administering the haplomers and bottle haplomers composition.

Pharmaceutical compositions may be administered by one of the following routes: oral, topical, systemic (e.g. transdermal, intranasal, or by suppository), or parenteral (e.g. intramuscular, subcutaneous, or intravenous injection). Compositions may take the form of tablets, pills, capsules, semisolids, powders, sustained release formulations, solutions, suspensions, elixirs, aerosols, or any other appropriate compositions; and comprise at least one compound in combination with at least one pharmaceutically acceptable excipient. Suitable excipients are well known to persons of ordinary skill in the art, and they, and the methods of formulating the compositions, may be found in such standard references as Remington: The Science and Practice of Pharmacy, A. Gennaro, ed., 20th edition, Lippincott, Williams & Wilkins, Philadelphia, Pa. Suitable liquid carriers, especially for injectable solutions, include water, aqueous saline solution, aqueous dextrose solution, and glycols.

Pharmaceutical compositions suitable for injection may include sterile aqueous solutions (where water soluble) or dispersions and sterile powders for the extemporaneous preparation of sterile injectable solutions or dispersion. In all cases, the composition should be sterile and should be fluid to the extent that easy syringeability exists. The composition should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents. In many cases, isotonic agents can be included, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition containing the haplomers and bottle haplomers in a suitable amount in an appropriate solvent with one or a combination of ingredients enumerated above. Generally, dispersions are prepared by incorporating the composition into a sterile vehicle which contains a basic dispersion medium and the required other ingredients from those enumerated above.

When the composition containing the haplomers and bottle haplomers is suitably protected, as described above, the composition can be formulated for oral administration, for example, with an inert diluent or an assimilable edible carrier. The composition and other ingredients can also be enclosed in a hard or soft shell gelatin capsule, compressed into tablets, or incorporated directly into the subject's diet. For oral therapeutic administration, the composition can be incorporated with excipients and used in the form of ingestible tablets, buccal tablets, troches, capsules, elixirs, suspensions, syrups, wafers, and the like. The percentage of the compositions and preparations can, of course, be varied. The amount of haplomers and bottle haplomers in such therapeutically useful compositions is such that a suitable dosage will be obtained.

It may be advantageous to formulate compositions in dosage unit form for ease of administration and uniformity of dosage. Each dosage unit form contains a predetermined quantity of the haplomers and bottle haplomers calculated to produce the amount of active effector product in association with a pharmaceutical carrier. The specification for the novel dosage unit forms is dependent on the unique characteristics of the targeted templated assembly composition, and the particular therapeutic effect to be achieved. Dosages are determined by reference to the usual dose and manner of administration of the ingredients.

The haplomers and bottle haplomers compositions may comprise pharmaceutically acceptable carriers, such that the carrier can be incorporated into the composition and administered to a patient without causing unacceptable biological effects or interacting in an unacceptable manner with other components of the composition. Such pharmaceutically acceptable carriers typically have met the required standards of toxicological and manufacturing testing, and include those materials identified as suitable inactive ingredients by the U.S. Food and Drug Administration.

The haplomers and bottle haplomers can also be prepared as pharmaceutically acceptable salts. Such salts can be, for example, a salt prepared from a base or an acid which is acceptable for administration to a patient, such as a mammal (e.g., salts having acceptable mammalian safety for a given dosage regime). However, it is understood that the salts covered herein are not required to be pharmaceutically acceptable salts, such as salts of the haplomers that are not intended for administration to a patient. Pharmaceutically acceptable salts can be derived from pharmaceutically acceptable inorganic or organic bases and from pharmaceutically acceptable inorganic or organic acids. In addition, when a haplomer contains both a basic moiety, such as an amine, and an acidic moiety such as a carboxylic acid, zwitterions may be formed and are included within the term “salt” as used herein. Salts derived from pharmaceutically acceptable inorganic bases can include ammonium, calcium, copper, ferric, ferrous, lithium, magnesium, manganic, manganous, potassium, sodium, and zinc salts, and the like. Salts derived from pharmaceutically acceptable organic bases can include salts of primary, secondary and tertiary amines, including substituted amines, cyclic amines, naturally-occurring amines and the like, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids can include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids can include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucoronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like.

The effector proteins generated by the processes described herein is the trigger that drives a desired action. Some examples of desired protein activity can include, but are not limited to, inducing an immune response, programmed cell death, apoptosis, non-specific or programmed necrosis, lysis, growth inhibition, inhibition of viral infection, inhibition of viral replication, inhibition of oncogene expression, modification of gene expression, inhibition of microbial infection, and inhibition of microbe replication, as well as combinations of these biological activities. In some embodiments, the protein produced can serve as a ligand for an antibody to induce an immune response at the site of the pathogenic cells, or to localize antibody-directed therapies, such as an antibody bearing a therapeutic payload, to the site of the pathogenic cells. In some embodiments, the protein produced can modulate expression of a target gene. In some embodiments, the protein produced can regulate enzyme activity, gene/protein expression, molecular signaling, and molecular interactions.

The following representative embodiments are presented:

Embodiment 1. A bottle haplomer comprising a polynucleotide, wherein the polynucleotide comprises: a) a first 3′ stem portion comprising from about 10 to about 20 nucleotide bases; b) an anti-target loop portion comprising from about 16 to about 40 nucleotide bases linked to the first 3′ stem portion, wherein the anti-target loop portion is substantially complementary to a target nucleic acid molecule; and c) a second 5′ stem portion comprising from about 10 to about 20 nucleotide bases linked to the anti-target loop portion, wherein the first 3′ stem portion is substantially complementary to the second 5′ stem portion; wherein: the 5′ terminus of the polynucleotide comprises a —SH moiety; and the Tₘ of the anti-target loop portion:target nucleic acid molecule is greater than the Tₘ of the first stem portion:second stem portion.

Embodiment 2. A bottle haplomer comprising a polynucleotide, wherein the polynucleotide comprises: a) a first 3′ stem portion comprising from about 10 to about 20 nucleotide bases; b) an anti-target loop portion comprising from about 16 to about 40 nucleotide bases linked to the first 3′ stem portion, wherein the anti-target loop portion is substantially complementary to a target nucleic acid molecule; and c) a second 5′ stem portion comprising from about 10 to about 20 nucleotide bases linked to the anti-target loop portion, wherein the first 3′ stem portion is substantially complementary to the second 5′ stem portion; wherein: the Tₘ of the anti-target loop portion:target nucleic acid molecule is greater than the Tₘ of the first stem portion:second stem portion; and the 5′ terminus or 3′ terminus of the polynucleotide is linked to the C-terminus of an N-terminal protein fragment or the N-terminus of a C-terminal protein fragment, wherein the terminus of the protein fragment lined to the polynucleotide comprises a cysteine or selenocysteine.

Embodiment 3. The bottle haplomer of embodiment 1 or embodiment 2 wherein the Tₘ of the first stem portion:second stem portion subtracted from the Tₘ of the anti-target loop portion:target nucleic acid molecule is from about 10° C. to about 40° C.

Embodiment 4. The bottle haplomer of any one of embodiments 1 to 3 wherein the Tₘ of the first stem portion:second stem portion is from about 40° C. to about 50° C.

Embodiment 5. The bottle haplomer of any one of embodiments 1 to 4 wherein the Tₘ of the anti-target loop portion:target nucleic acid molecule is from about 60° C. to about 80° C.

Embodiment 6. The bottle haplomer of any one of embodiments 1 to 5 wherein the Tₘ of the first stem portion:second stem portion subtracted from the Tₘ of the anti-target loop portion:target nucleic acid molecule is from about 10° C. to about 20° C.

Embodiment 7. The bottle haplomer of any one of embodiments 1 to 6 wherein the first stem portion comprises from about 12 to about 18 nucleotide bases.

Embodiment 8. The bottle haplomer of any one of embodiments 1 to 7 wherein the anti-target loop portion comprises from about 18 to about 35 nucleotide bases.

Embodiment 9. The bottle haplomer of any one of embodiments 1 to 8 wherein the second stem portion comprises from about 12 to about 18 nucleotide bases.

Embodiment 10. The bottle haplomer of any one of embodiments 1 to 9 wherein the nucleotide bases of any one or more of the first stem portion, anti-target loop portion, and second stem portion are selected from the group consisting of DNA nucleotides, RNA nucleotides, phosphorothioate-modified nucleotides, 2-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, DNA analogs with L-ribose (L-DNA), Xeno nucleic acid (XNA) analogues, or other nucleic acid analogues capable of base-pair formation, or artificial nucleic acid analogues with altered backbones, or any combination thereof.

Embodiment 11. The bottle haplomer of any one of embodiments 1 to 10 further comprising a linker between any one or more of the first stem portion and the anti-target loop portion, or between the anti-target loop portion and the second stem portion.

Embodiment 12. The bottle haplomer of embodiment 11 wherein the linker is selected from the group consisting of an alkyl group, an alkenyl group, an amide, an ester, a thioester, a ketone, an ether, a thioether, a disulfide, an ethylene glycol, a cycloalkyl group, a benzyl group, a heterocyclic group, a maleimidyl group, a hydrazone, a urethane, azoles, an imine, a haloalkyl, and a carbamate, or any combination thereof.

Embodiment 13. A haplomer comprising: a) a polynucleotide; and b) an N-terminal protein fragment or a C-terminal protein fragment, wherein the 3′ or 5′ terminus of the polynucleotide is linked to the N-terminus of the C-terminal protein fragment or the C-terminus of the N-terminal protein fragment; wherein: the N-terminal fragment comprises the amino acid sequence of APIVTCRKLDGREKPFKVDVATAQAQARKAGLTTGKSGDPHRYFAGDHIR WGVNNCDKADAILWEYPIYWVGKNAEWAKDVKTSQQKG (SEQ ID NO:1), and the C-terminal fragment comprises the amino acid sequence of GPTPIRVVYANSRGAVQYCGVM THSKVDKNNQGKEFFEKCD (SEQ ID NO:2); the N-terminal fragment comprises the amino acid sequence of APIVTCRPKLDG (SEQ ID NO:3), and the C-terminal fragment comprises the amino acid sequence of REKPFKVDVATAQAQARKAGLTTGKSGDPHRYFAGDHIRWGVN NCDKADAILWEYPIYWVGKNAEWAKDVKTSQQKGGPTPIRVVYANSRGAVQYCGVM THSKVDKNNQGKEFFEKCD (SEQ ID NO:4); the N-terminal fragment comprises the amino acid sequence of APIVTCRPKLDGREKPFKVDVATAQAQARKAGLTTGK (SEQ ID NO:5), and the C-terminal fragment comprises the amino acid sequence of SGDPHRYFAGDHIRWGV NNCDKADAILWEYPIYWVGKNAEWAKDVKTSQQKGGPTPIRVVYANSRGAVQYCGV MTHSKVDKNNQGKEFFEKCD (SEQ ID NO:6); the N-terminal fragment comprises the amino acid sequence of APIVTCRPKLDGREKPFKVDVATAQAQARKAGLTTGKSGDPH RYFAGDHIRWGVNNCDKAD (SEQ ID NO:7), and the C-terminal fragment comprises the amino acid sequence of AILWEYPIYWVGKNAEWAKDVKTSQQKGGPTPIRVVYANSRGA VQYCGVMTHSKVDKNNQGKEFFEKCD (SEQ ID NO:8); the N-terminal fragment comprises the amino acid sequence of APIVTCRPKLDGREKPFKVDVATAQAQARKAGLT TGKSGDPHRYFAGDHIRWGVNNCDKADAILWEYPIYWVG (SEQ ID NO:9), and the C-terminal fragment comprises the amino acid sequence of KNAEWAKDVKTSQQKGGPTPIR VVYANSRGAVQYCGVMTHSKVDKNNQGKEFFEKCD (SEQ ID NO:10); the N-terminal fragment comprises the amino acid sequence of APIVTCRPKLDGREKPFKVDVATAQAQA RKAGLTGKSGDPHRYFAGDHIRWGVNNCDKADAILWEYPIYWVGKNAEWAKD (SEQ ID NO:11), and the C-terminal fragment comprises the amino acid sequence of VKTSQQKGGP TPIRVVYANSRGAVQYCGVMTHSKVDKNNQGKEFFEKCD (SEQ ID NO:12); the N-terminal fragment comprises the amino acid sequence of APIVTCRPKLDGREKPFKVDVAT AQAQARKAGLTGKSGDPHRYFAGDHIRWGVNNCDKADAILWEYPIYWVGKNAEWA KDVKTSQ (SEQ ID NO:13), and the C-terminal fragment comprises the amino acid sequence of QKGGPTPIRVVYANSRGAVQYCGVMTHSKVDKNNQGKEFFEKCD (SEQ ID NO:14); the N-terminal fragment comprises the amino acid sequence of APIVTCRPKLDGREKPFKVD VATAQAQARKAGLTTGKSGDPHRYFAGDHIRWGVNNCDKADAILWEYPIYWVGKNA EWAKDVKTSQQKGGPTPIRVVYANSRG (SEQ ID NO:15), and the C-terminal fragment comprises the amino acid sequence of AVQYCGVMTHSKVDKNNQGKEFFEKCD (SEQ ID NO:16); the N-terminal fragment comprises the amino acid sequence of APIVTCRPKLDGREK PFKVDVATAQAQARKAGLTTGKSGDPHRYFAGDHIRWGVNNCDKADAILWEYPIYWV GKNAEWAKDVKTSQQKGGPTPIRVVYANSRGAVQYCGVMTHSKVDKN (SEQ ID NO:17), and the C-terminal fragment comprises the amino acid sequence of NQGKEFFEKCD (SEQ ID NO:18); or the N-terminal fragment comprises the amino acid sequence of APIVTCRP KLDGREKPFKVDVATAQAQARKAGLT; (SEQ ID NO:40), and the C-terminal fragment comprises the amino acid sequence of TGKSGDPHRYFAGDHIRWGVNNCDKADAILWEYPI YWVGKNAEWAKDVKTSQQKGGPTPIRVVYANSRGAVQYCGVMTHSKVDKNNQGKEF FEKCD (SEQ ID NO:41).

Embodiment 14. A surface target compound comprising: a) a template polynucleotide; and b) a peptide; wherein: the 5′ terminus of the polynucleotide is coupled to the N-terminus or C-terminus of the peptide, or the 3′ terminus of the polynucleotide is coupled to the N-terminus or C-terminus of the peptide; and the peptide is a ligand for a cell-surface molecule.

Embodiment 15. The surface target compound of embodiment 14 wherein the ligand is a peptide hormone or a neuropeptide.

Embodiment 16. The surface target compound of embodiment 15 wherein the peptide hormone is selected from the group consisting of alpha-MSH, amylin, anti-Müllerian hormone, adiponectin, atriopeptide, human growth hormone, gonadotropin releasing hormone, inhibin, somatostatin, adrenocorticotropic hormone, vasopressin, vasoactive intestinal peptide, gastrin, secretin, gastric inhibitory polypeptide, motilin, hepcidin, renin, relaxin, ghrelin, leptin, lipotropin, angiotensin I, angiotensin 11, bradykinin, calcitonin, insulin, glucagon, insulin-like growth factor 1, insulin-like growth factor II, glucagon-like peptide 1, pancreatic polypeptide, betatrophin, cholecystokinin, endothelin, erythropoietin, thrombopoietin, follicle-stimulating hormone, human chorionic gonadotropin, human placental lactogen, prolactin, prolactin releasing hormone, luteinizing hormone, thyroid-stimulating hormone, thyrotropin-releasing hormone, parathyroid hormone, and pituitary adenylate cyclase-activating peptide.

Embodiment 17. The surface target compound of embodiment 15 wherein the neuropeptide is selected from the group consisting of neuropeptide Y, an endorphin, an encephalin, brain natriuretic peptide, tachykinin, cortistatin, galanin, orexin, and oxytocin.

Embodiment 18. The surface target compound of embodiment 14, wherein the polynucleotide comprises the nucleotide sequence AAGCCACTGTGTCCTGAAGAAAAGCA AAGACATC (SEQ ID NO:20), and the peptide comprises the amino acid sequence SYSMEHFRWGKPVGGGSSGGGC (SEQ ID NO:21), SYSXEHFRWGKPVGGGSSGGGC (SEQ ID NO:22), CSGGGSSGGGSYSMEHFRWGKPV-NH₂ (SEQ ID NO:23), or CSGGGSSGGGSYSXEHFRWGKPV-NH₂ (SEQ ID NO:24), wherein X is norleucine and the F residue is D-phenylalanine.

Embodiment 19. A fusion protein comprising: an N-terminal protein fragment, a fusion partner protein, and a purification domain, wherein the C-terminus of the N-terminal protein fragment is coupled to the N-terminus of the fusion partner protein, and the C-terminus of the fusion partner protein is coupled to the N-terminus of the purification domain; or an N-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the N-terminal protein fragment, wherein the N-terminal protein fragment comprises an N-terminal methionine and a C-terminal cysteine; or a C-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the C-terminal protein fragment, wherein the C-terminal protein fragment comprises an N-terminal cysteine.

Embodiment 20. The fusion protein of embodiment 19 comprising: an N-terminal protein fragment, intein, and a chitin-binding domain, wherein the C-terminus of the N-terminal protein fragment is coupled to the N-terminus of intein, and the C-terminus of intein is coupled to the N-terminus of the chitin-binding domain; or an N-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the N-terminal protein fragment, wherein the N-terminal protein fragment comprises an N-terminal methionine and a C-terminal cysteine; or a C-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the C-terminal protein fragment, wherein the C-terminal protein fragment comprises an N-terminal cysteine.

Embodiment 21. The fusion protein of embodiment 20 comprising an N-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the N-terminal protein fragment, wherein the N-terminal protein fragment comprises the amino acid sequence APIVTCRPKLDGREKPFKVDVATAQAQARKAGLTTGKSGDPHR YFAGDHIRWGVNNCDKADAILWEYPIYWVGKNAEWAKDVKTSQQKGC (SEQ ID NO:25).

Embodiment 22. The fusion protein of embodiment 19 comprising a C-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the C-terminal protein fragment, wherein the C-terminal protein fragment comprises the amino acid sequence

(SEQ ID NO: 26)
CGPTPIRVVYANSRGAVQYCGVMTHSKVDKNNQGKEFFEKCD.

Embodiment 23. A compound having the formula

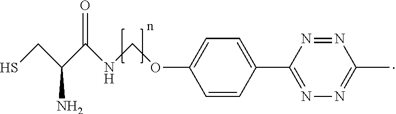

wherein n is from about 3 to about 6.

Embodiment 24. A composition or kit comprising: a) a first haplomer, wherein the first haplomer comprises a polynucleotide linked to the C-terminus of an N-terminal protein fragment; and b) a second haplomer, wherein the second haplomer comprises a polynucleotide linked to the N-terminus of a C-terminal protein fragment; wherein: the polynucleotide of one of the first or second haplomers is linked at its 5′ terminus to the protein fragment, and the other of the first and second haplomers is linked at its 3′ terminus to the protein fragment; the N-terminal protein fragment and the C-terminal protein fragment are derived from a single protein; and wherein: the polynucleotide of the first haplomer is complementary to the polynucleotide of the second haplomer; or the polynucleotide of the first haplomer is complementary to a target nucleic acid molecule, and the polynucleotide of the second haplomer is substantially complementary to the target nucleic acid molecule at a site in spatial proximity to the polynucleotide of the first haplomer; or the polynucleotide of the first haplomer is substantially complementary to a portion of a target nucleic acid molecule 5′ adjacent to a stem-loop structure, and the polynucleotide of the second haplomer is substantially complementary to a portion of the target nucleic acid molecule 3′ adjacent to the stem-loop structure; or the polynucleotide of the first haplomer is substantially complementary to a 5′ portion of a loop of a stem-loop structure of a target nucleic acid molecule, and the polynucleotide of the second haplomer is substantially complementary to a 3′ portion of the loop of the stem-loop structure of the target nucleic acid molecule.

Embodiment 25. A composition or kit comprising: a) a bottle haplomer comprising a polynucleotide comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein: the 5′ terminus of the polynucleotide comprises a —SH moiety; and the Tₘ of the anti-target loop portion:target nucleic acid molecule is greater than the Tₘ of the first stem portion:second stem portion; b) an N-terminal protein fragment, wherein the C-terminus of the N-terminal protein fragment comprises a cysteine-SH moiety; and c) a bis-maleimide reagent.

Embodiment 26. A composition or kit comprising: a) a bottle haplomer comprising a polynucleotide comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and b) a second haplomer comprising a polynucleotide and a C-terminal protein fragment, wherein the 3′ terminus of the polynucleotide is linked to the N-terminus of the C-terminal protein fragment, wherein the N-terminus comprises a cysteine; wherein: the polynucleotide of the second haplomer is substantially complementary to the second 5′ stem portion of the polynucleotide of the bottle haplomer; the Tₘ of the anti-target loop portion:target nucleic acid molecule is greater than the Tₘ of the first stem portion:second stem portion; and the N-terminal protein fragment and the C-terminal protein fragment are derived from a single protein.

Embodiment 27. The kit or composition of any one of embodiments 24 to 26 wherein the nucleotide bases of the haplomer, or any one or more of the first stem portion, anti-target loop portion, and second stem portion of the bottle haplomer are selected from the group consisting of DNA nucleotides, RNA nucleotides, phosphorothioate-modified nucleotides, 2-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, DNA analogs with L-ribose (L-DNA), Xeno nucleic acid (XNA) analogues, or other nucleic acid analogues capable of base-pair formation, or artificial nucleic acid analogues with altered backbones, or any combination thereof.

Embodiment 28. The kit or composition of embodiment 25 or embodiment 26 wherein the Tₘ of the first stem portion:second stem portion subtracted from the Tₘ of the anti-target loop portion:target nucleic acid molecule is from about 10° C. to about 40° C.

Embodiment 29. The kit or composition of any one of embodiments 25 to 28 wherein the Tₘ of the first stem portion:second stem portion is from about 40° C. to about 50° C.

Embodiment 30. The kit or composition of any one of embodiments 25 to 29 wherein the Tₘ of the anti-target loop portion:target nucleic acid molecule is from about 60° C. to about 80° C.

Embodiment 31. The kit or composition of any one of embodiments 25 to 30 wherein the Tₘ of the first stem portion:second stem portion subtracted from the Tₘ of the anti-target loop portion:target nucleic acid molecule is from about 10° C. to about 20° C.

Embodiment 32. The kit or composition of any one of embodiments 25 to 31 wherein the first stem portion comprises from about 12 to about 18 nucleotide bases.

Embodiment 33. The kit or composition of any one of embodiments 25 to 32 wherein the anti-target loop portion comprises from about 18 to about 35 nucleotide bases.

Embodiment 34. The kit or composition of any one of embodiments 25 to 33 wherein the second stem portion comprises from about 12 to about 18 nucleotide bases.

Embodiment 35. The kit or composition of embodiment 26 wherein the Tₘ of the duplex formed by the second haplomer and the first or second stem portion of the bottle haplomer subtracted from the Tₘ of the first stem portion:second stem portion is from about 0° C. to about 20° C.

Embodiment 36. The kit or composition of embodiment 26 wherein the Tₘ of the duplex formed by the second haplomer and the first or second stem portion of the bottle haplomer is from about 30° C. to about 40° C.

Embodiment 37. The kit or composition of embodiment 26 wherein the Tₘ of the duplex formed by the second haplomer and the first or second stem portion of the bottle haplomer subtracted from the Tₘ of the first stem portion:second stem portion is from about 5° C. to about 10° C.

Embodiment 38. The kit or composition of any one of embodiments 24 to 37 wherein the polynucleotide and protein fragment each comprise a bio-orthogonal reactive molecule.

Embodiment 39. The kit or composition of embodiment 38 wherein the bio-orthogonal reactive molecule is an azide, an alkyne, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, or a quadricyclane, or any derivative thereof.

Embodiment 40. The kit or composition of any one of embodiments 25 to 39 further comprising a linker between the first stem portion and the anti-target loop portion or between the anti-target loop portion and the second stem portion.

Embodiment 41. The kit or composition of embodiment 40 wherein the linker is an alkyl group, an alkenyl group, an amide, an ester, a thioester, a ketone, an ether, a thioether, a disulfide, an ethylene glycol, a cycloalkyl group, a benzyl group, a heterocyclic group, a maleimidyl group, a hydrazone, a urethane, azoles, an imine, a haloalkyl, nitrilotriacetic acid, nickel, cobalt, copper, and a carbamate, or any combination thereof.

Embodiment 42. The kit or composition of any one of embodiments 25 to 41 wherein the anti-target loop portion further comprises an internal hinge region, wherein the hinge region comprises one or more nucleotides that are not complementary to the target nucleic acid molecule.

Embodiment 43. The kit or composition of embodiment 42 wherein the hinge region comprises from about 1 nucleotides to about 6 nucleotides.

Embodiment 44. The haplomer, bottle haplomer, fusion protein, or kit or composition of any one of embodiments 1 to 14, 19 to 22, or 24 to 43 wherein the N-terminal protein fragment and C-terminal protein fragment are both derived from a reporter protein, a transcription factor, a signal transduction pathway factor, a gene editing protein, a single-chain immunoglobulin variable region (scFv) protein, a toxic protein, or an enzyme.

Embodiment 45. The haplomer, bottle haplomer, fusion protein, or kit or composition of embodiment 44 wherein enzyme is a 8-lactamase, a choramphenicol acetyl transferase, an aminoglycoside-3′-phosphotransferase, 8-galactosidase, a dihydrofolate reductase, a restriction enzyme, a DNase, or an RNase.

Embodiment 46. The haplomer, bottle haplomer, fusion protein, or kit or composition of embodiment 44 wherein the reporter protein is a fluorescent protein, a luciferase, a choramphenicol acetyl transferase, a 8-galactosidase, or a 8-glucuronidase.

Embodiment 47. The haplomer, bottle haplomer, fusion protein, or kit or composition of embodiment 46 wherein the fluorescent protein is GFP, YFP, mCherry, dsRed, VENUS, or CFP, a blue fluorescent protein, or any analog thereof.

Embodiment 48. The haplomer, bottle haplomer, fusion protein, or kit or composition of embodiment 46 wherein the fluorescent protein is superfolder GFP.

Embodiment 49. The haplomer, bottle haplomer, fusion protein, or kit or composition of embodiment 48 wherein N-terminal fragment of the superfolder GFP comprises the amino acid sequence of MSKGEELFTGVVPILVELDGDVNGHKFSVRGEGEGDATNGKLTLKFICTTG KLPVPWPTLVTTLTYGVQCFSRYPDHMKRHDFFKSAMPEGYVQERTISFKDDGTYKTR AEVKFEGDTLVNRIELKGIDFKEDGNILGHKLEYNFNSHNVYITADKQ (SEQ ID NO:33).

Embodiment 50. The haplomer, bottle haplomer, fusion protein, or kit or composition of embodiment 48 wherein C-terminal fragment of the superfolder GFP comprises the amino acid sequence of KNGIKANFKIRHNVEDGSVQLADHYQQNTPIGDGPVLLPDNHYLSTQSVLS KDPNEKRDHMVLLEFVTAAGITHGMDELYK (SEQ ID NO:34).

Embodiment 51. The haplomer, bottle haplomer, fusion protein, or kit or composition of embodiment 46 wherein the luciferase is firefly luciferase, Renilla luciferase, or Gaussia princeps luciferase.

Embodiment 52. The haplomer, bottle haplomer, fusion protein, or kit or composition of embodiment 51 wherein the luciferase is Renilla luciferase.

Embodiment 53. The haplomer, bottle haplomer, fusion protein, or kit or composition of embodiment 52 wherein N-terminal fragment of the Renilla luciferase comprises the amino acid sequence of MASKVYDPEQRKRMITGPQWWARCKQMNVLDSFINYYDSEKHAENAVIF LHGNAASSYLWRHVVPHIEPVARCIIPDLIGMGKSGKSGNGSYRLLDHYKYLTAWFELL NLPKKIIFVGHDWGACLAFHYSYEHQDKIKAIVHAESVVDVIESWDEWPDIEEDIALIKS EEGEKMVLENNFFVETMLPSKIMRKLEPEEFAAYLEPFKEKGEVRRPTLSWPREIPLVKG GY (SEQ ID NO:36).

Embodiment 54. The haplomer, bottle haplomer, fusion protein, or kit or composition of embodiment 52 wherein C-terminal fragment of the Renilla luciferase comprises the amino acid sequence of KPDVVQIVRNYNAYLRASDDLPKMFIESDPGFFSNAIVEGAKKFPNTEFVKV KGLHFSQEDAPDEMGKYIKSFVERVLKNEQZ (SEQ ID NO:37).

Embodiment 55. The haplomer, bottle haplomer, fusion protein, or kit or composition of embodiment 44 wherein the toxic protein is ricin A chain, Aspf1, α-sarcin, mitogillin, hirsutellin A, diphtheria toxin, botulinum A toxin, or cholera toxin.

Embodiment 56. The haplomer, bottle haplomer, fusion protein, or kit or composition of embodiment 44 wherein the toxic protein is a ribotoxin that cleaves the large 28S ribosomal RNA.

Embodiment 57. The haplomer, bottle haplomer, fusion protein, or kit or composition of any one of embodiments 1 to 14, 19 to 22, or 24 to 43 wherein the target nucleic acid molecule is a cellular nucleic acid molecule, a tumor-specific nucleic acid molecule, an aberrant immune pathway nucleic acid molecule, or the polynucleotide of a surface target compound.

Embodiment 58. The composition or kit of any one of embodiments 24 to 43 further comprising a protein chaperone, a small-molecule chaperone, or a pharmacoperone.

Embodiment 59. The composition or kit of embodiment 58 wherein the protein chaperone is a heat-shock protein.

Embodiment 60. The composition or kit of embodiment 58 wherein the small-molecule chaperone is 4-phenyl butyrate, deoxycholic acid, ursodeoxycholic acid, taurourso-deoxycholic acid, lysophosphatidic acid, trehalose, mannitol, trimethylamine oxide, betaine, or dimethylsulfoxide.

Embodiment 61. The fusion protein of any one of embodiments 19 to 22 wherein the fusion partner protein is intein, a maltose-binding protein, glutathione-S-transferase, 8-galactosidase, or Omp F.

Embodiment 62. The fusion protein of any one of embodiments 19 to 22 wherein the cleavage site is an enterokinase cleavage site or a Factor Xa protease cleavage site.

Embodiment 63. The fusion protein of embodiment 62 wherein the Factor Xa protease cleavage site is IEGR (SEQ ID NO:27).

Embodiment 64. The fusion protein of any one of embodiments 19 to 22 wherein the purification domain is a chitin-binding domain or a hexahistidine tag.

Embodiment 65. A method for the directed assembly of a protein in a cell comprising: a) contacting a cell with a first haplomer comprising a polynucleotide linked to the C-terminus of an N-terminal protein fragment; and b) contacting the cell with a second haplomer comprising a polynucleotide linked to the N-terminus of a C-terminal protein fragment; wherein: the polynucleotide of one of the first or second haplomers is linked at its 5′ terminus to the protein fragment, and the other of the first and second haplomers is linked at its 3′ terminus to the protein fragment; the N-terminal protein fragment and the C-terminal protein fragment are derived from a single protein; and wherein: the polynucleotide of the first haplomer is substantially complementary to the polynucleotide of the second haplomer; or the polynucleotide of the first haplomer is substantially complementary to a target nucleic acid molecule, and the polynucleotide of the second haplomer is substantially complementary to the target nucleic acid molecule at a site in spatial proximity to the polynucleotide of the first haplomer; or the polynucleotide of the first haplomer is substantially complementary to a portion of a target nucleic acid molecule 5′ adjacent to a stem-loop structure, and the polynucleotide of the second haplomer is substantially complementary to a portion of the target nucleic acid molecule 3′ adjacent to the stem-loop structure; or the polynucleotide of the first haplomer is substantially complementary to a 5′ portion of a loop of a stem-loop structure of a target nucleic acid molecule, and the polynucleotide of the second haplomer is substantially complementary to a 3′ portion of the loop of the stem-loop structure of the target nucleic acid molecule; thereby resulting in the assembly of the protein from the N-terminal protein fragment and the C-terminal protein fragment.

Embodiment 66. A method for the directed assembly of a protein comprising: a) contacting a target nucleic acid molecule with a bottle haplomer comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a target nucleic acid molecule; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and b) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer; wherein: the N-terminal protein fragment and the C-terminal protein fragment are derived from a single protein; the Tₘ of the anti-target loop portion:target nucleic acid molecule is greater than the Tₘ of the first stem portion:second stem portion; and the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

Embodiment 67. A method for the directed assembly of a protein comprising: a) contacting a cell with a surface target compound comprising: i) a template polynucleotide; and ii) a peptide; wherein: the 5′ terminus of the polynucleotide is coupled to the N-terminus or C-terminus of the peptide, or the 3′ terminus of the polynucleotide is coupled to the N-terminus or C-terminus of the peptide; and the peptide is a ligand for a cell-surface molecule; b) contacting the cell with a first haplomer comprising a polynucleotide linked to the C-terminus of an N-terminal protein fragment; and c) contacting the cell with a second haplomer comprising a polynucleotide linked to the N-terminus of a C-terminal protein fragment; wherein: the polynucleotide of one of the first or second haplomers is linked at its 5′ terminus to the protein fragment, and the other of the first and second haplomers is linked at its 3′ terminus to the protein fragment; the N-terminal protein fragment and the C-terminal protein fragment are derived from a single protein; and the polynucleotide of the first haplomer is substantially complementary to the template polynucleotide of the surface target compound, and the polynucleotide of the second haplomer is substantially complementary to the template polynucleotide of the surface target compound at a site in spatial proximity to the polynucleotide of the first haplomer; thereby resulting in the assembly of the protein from the N-terminal protein fragment and the C-terminal protein fragment.

Embodiment 68. A method for the directed assembly of a protein comprising: a) contacting a cell with a surface target compound comprising: i) a template polynucleotide; and ii) a peptide; wherein: the 5′ terminus of the polynucleotide is coupled to the N-terminus or C-terminus of the peptide, or the 3′ terminus of the polynucleotide is coupled to the N-terminus or C-terminus of the peptide; and the peptide is a ligand for a cell-surface molecule; b) contacting a target nucleic acid molecule with a bottle haplomer comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to the template polynucleotide of the surface target compound; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and c) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer; wherein: the N-terminal protein fragment and the C-terminal protein fragment are derived from a single protein; the Tₘ of the anti-target loop portion:target nucleic acid molecule is greater than the Tₘ of the first stem portion:second stem portion; and the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

Embodiment 69. A method of cleaving an N-terminal protein fragment from an intein fusion partner in a fusion protein comprising: a) contacting the fusion protein with 2-mercaptoethane sulfonic acid; and b) contacting the fusion protein with a cysteine having a methyltetrazine group; thereby releasing the N-terminal protein fragment from the fusion protein.

Embodiment 70. The method of embodiment 69 wherein the cysteine having a methyltetrazine group is

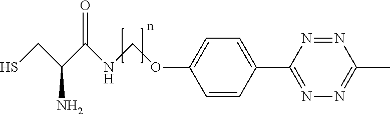

Embodiment 71. The method of embodiment 69 further comprising reacting the N-terminal protein fragment with a polynucleotide having a 5′ or 3′ trans-cyclooctene group.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Protein Solubilities in an Inteln-Based System—Expression of N-Terminal sfGFP Fragment

Implementation of SP-TAPER provides the expression of predetermined polypeptide fragments of a whole protein of interest, prior to conjugation with nucleic acid tags. For this purpose, a suitable bacterial expression system is evaluated for the split-protein fragments needed. One aspect of successful expression in prokaryotic systems is the maintenance of protein solubility. Although insoluble inclusion bodies can often be resolubilized, it is preferable to avoid this time-consuming step if possible.

Two separate reporter proteins were considered for initial SP-TAPER: sfGFP and Renilla luciferase. The sfGFP protein was divided into N-terminal and C-terminal fragments of 157 and 81 amino acid residues, respectively, at the site of a loop region. Renilla luciferase was divided into N-terminal and C-terminal fragments of 229 and 81 residues, respectively, based on a previous screen for cleavage sites compatible with the conventional protein complementation assay (Paulmurugan et al., Anal. Chem., 203, 75, 1584-1589).

In both the sfGFP and Renilla luciferase model systems, the chosen N-terminal fragments were significantly longer than their corresponding C-terminal fragments. While protein fragment insolubility in prokaryotic expression systems is subject to multiple factors, the longer the expressed fragment, the greater the likelihood of inclusion of hydrophobic tracts (normally packed with the correctly folded full-length protein) and encountering solubility problems. Accordingly, the longer sfGFP and Renilla N-terminal fragments were initially examined in an intein-based expression system (New England Biolabs). The coding sequence for each fragment, optimized for expression in E. coli, was inserted into the Nde I/Sap I cloning sites of the intein-based expression vector pTXB1 (New England Biolabs), such that the correct junction sequences and reading frames were produced, where the desired coding sequence was cloned as a 5′ in-frame fusion with cleavable intein domain sequence, in turn fused with coding sequence for an affinity-selectable chitin-binding domain (confirmed by sequencing of candidate clones).

Verified plasmid clones were transected into the E. coli strain T7 express (New England Biolabs) and propagated in liquid culture (50 ml) under short-term growth conditions at 37° C. for 1.5 hours, before induction with 400 μM IPTG for a further 2 hours. Samples (200 μl “direct lysates”) were obtained, pelleted in 1.5 ml tubes at 1000 g, washed once with 200 μl of 1×PBS, and resuspended in 50 μl of PBS. The remainder of the 50 ml growths were pelleted (10 minutes at 3000 rpm in a Sorvall benchtop centrifuge), and resuspended in 2.0 ml Eppendorf tubes in 1.5 ml of ice-cold TXB-column buffer (20 mM HEPES pH 8.5, 500 mM NaCl, and 0.05% Triton-X100), with 1% protease inhibitor cocktail (Sigma P3840). Cell suspensions were then sonicated (6×5 second pulses, 5-setting, Branson 450 Sonifier, with chilling between each sonication round), centrifuged 5 minutes at 14000 rpm (benchtop microfuge), and the supernatants removed to a fresh tube.

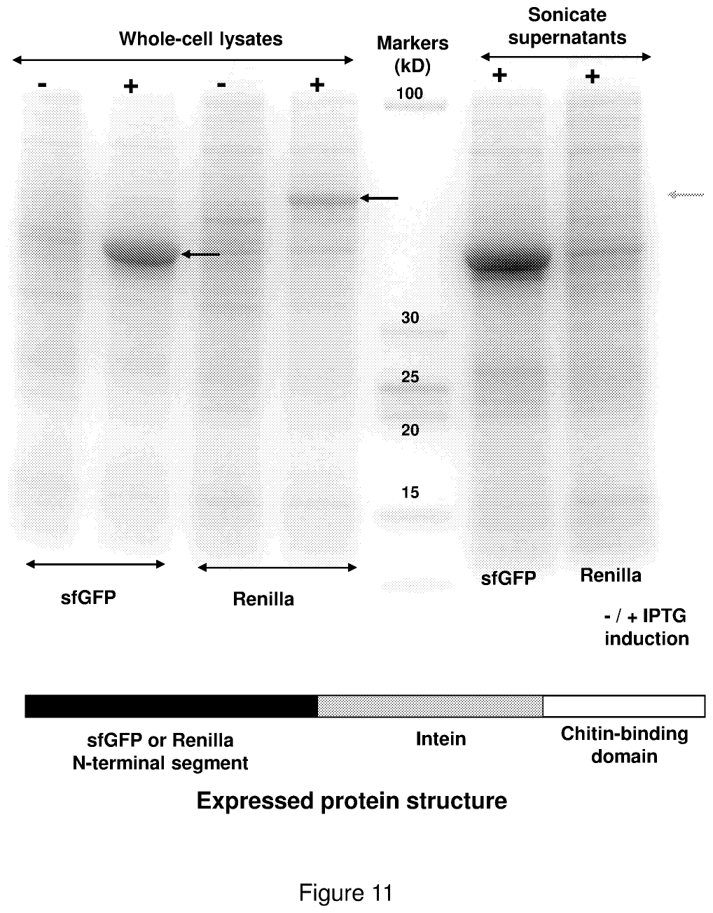

Samples of supernatants and the direct lysate samples (50 μl) described above were mixed with an equal volume of standard 2×Laemmli SDS-PAGE lysis buffer (Bio-Rad), and the samples heated at 100° C. for 5 minutes. These were then loaded onto an SDS-PAGE gel (5 μl/lane; “any-kD” TGX gel, Bio-Rad), fixed, and stained overnight with SYPRO-Ruby (Thermo). Following destaining, gels were visualized with a UV transilluminator. Both sfGFP and Renilla luciferase N-terminal fusion fragments were observed at the expected molecular weights in whole-cell lysate samples of IPTG-induced cultures (see, FIG. 11), although the Renilla band intensity was considerably less than for sfGFP. After sonication, the sfGFP band was readily observable in cleared supernatants, but no Renilla band was observable. These results showed that this intein-based system is poorly compatible with Renilla fragment expression under the conditions used. On the other hand, the sfGFP N-terminal fragment was soluble and expressed at good yield, and compatible with further processing towards preparing specific conjugates.

### Example 2: Affinity Purification of N-Terminal sfGFP-Intein Fusion and Intein-Mediated Cleavage from Solid-Phase

The solubility of the N-terminal sfGFP fragment when expressed as a fusion protein in the intein-based system (described in Example 1) indicated that it was appropriate to examine further for preparation of the free N-terminal fragment itself. By means of the chitin-binding domain segment of the fusion protein (see, FIG. 11), the soluble N-terminal sfGFP fusion fragment in whole-cell sonicated supernatants was bound to chitin magnetic beads (CMBs; New England Biolabs) in the following manner: duplicate 50 ml growths of plasmids encoding the N-terminal sfGFP-intein-chitin binding domain fusion in the strain T7 Express were propagated, induced with 100 μM IPTG, and whole-cell lysate samples were obtained. Sonicated clarified supernatants were subsequently prepared; these initial steps were performed in a similar manner as for Example 1. A suitable quantity of CMBs (2 tubes with 100 μl bead slurries each) were washed twice (using magnetic separation of the beads) with 0.5 ml of ice-cold TXB buffer (see, Example 1), and then resuspended in TXB buffer in the original volume (100 μl). To each tube, 1.25 ml of the induced sonicated supernatants for the N-terminal sfGFP fusion protein were added, and incubated at 4° C. for 1 hour with frequent tube inversions for mixing. Following this, the beads were magnetically separated, the supernatants removed, and the beads subjected to three washings with TXB buffer, before finally pooling into a suspension in the same buffer at a final total volume of 200 μl.

Material remaining on the chitin magnetic beads via the chitin-binding domain of the fusion protein was then subjected to a series of treatments to examine optimal means for preparing the isolated N-terminal sfGFP fragment. In this system, activation of the intein at the insert polypeptide junction with appropriate thiol reagents can result in the cleavage and release of the desired polypeptide fragment, while the intein-chitin binding domain segment remains bound to the chitin beads. The reagent 2-mercaptoethane sulfonic acid (MESNA) is frequently used for this purpose. The solubility and other properties of the intein cleavage products can be modulated by varying the sodium chloride concentration. Accordingly, the chitin beads bearing the N-terminal sfGFP fusion were tested with several MESNA/salt conditions with a 16 hour incubation at 25° C. For each experimental condition, 20 μl of the washed chitin magnetic bead/fusion protein slurries were used in a total volume of 40 μl. At the end of the incubation period, supernatants were magnetically removed. The bead pellets were retained and washed twice with 0.5 ml of TXB buffer before reconstitution in 30 μl of the same buffer. In all cases, 25 or 30 μl of samples were mixed with an equal volume of 2× Laemmli SDS-PAGE loading buffer, and heated for 5 minutes at 100° C., before loading 5 μl samples onto SDS-PAGE gels.

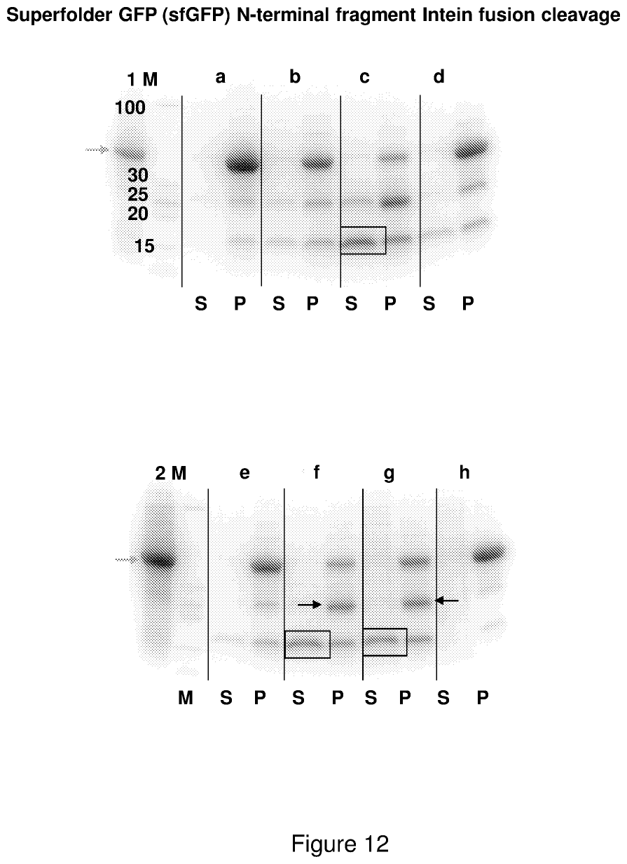

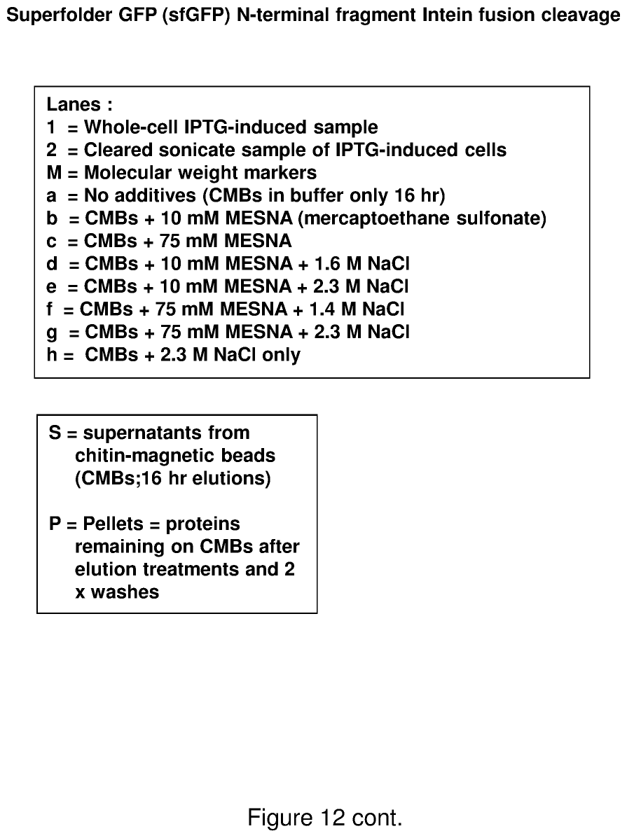

In this representative Example, induction of the sfGFP fusion in whole-cell lysates, and derivation of supernatants containing the soluble fusion were achieved (see, Lanes 1 and 2, FIG. 12). Following an overnight incubation of the chitin beads bearing the N-terminal sfGFP fusion in TCB buffer, no non-specific elution of protein was observed, although a low level of spontaneously cleaved intein-chitin binding domain (about 28 kD) and N-terminal sfGFP (about 17 kD) remained associated with the beads (see, Lanes aS/aP; FIG. 12). A higher concentration of MESNA than the 10 mM commonly used (New England Biolabs) was more effective at producing cleaved N-terminal sfGFP fragment in the soluble supernatant (see, Lane set b (10 mM MESNA) vs. Lane set c (75 mM MESNA)). During these MESNA incubations, a significant amount of the intein-chitin binding domain leached off the beads as well as the expected released N-terminal sfGFP fragment. However, this undesirable effect was suppressible by means of higher sodium chloride concentrations (see, Lane set f (75 mM MESNA/1.4 M NaCl) vs. Lane set g (75 mM MESNA/2.3 M NaCl)).

These results indicate that the N-terminal sfGFP fragment can be successfully prepared by means of the intein-based system and, moreover, affords a C-terminal conjugation strategy with an oligonucleotide, by means of a modified cysteine bearing a methyltetrazine group, as described above.

### Example 3: Expression and Purification of C-Terminal sfGFP and Renilla Fragments as Maltose-Binding Protein Fusions, and Fragment Cleavage with Enterokinase

For expression of the C-terminal fragments of both sfGFP and Renilla luciferase and labeling of the N-termini (N*) of such products, an alternative expression system with maltose-binding protein was technically more convenient than the intein system of Examples 1 and 2. In addition, in the case of the model proteins sfGFP and Renilla luciferase, neither possesses a cysteine residue within the chosen C-terminal segments, rendering oligonucleotide conjugation via insertion of an N-terminal cysteine a facile option.

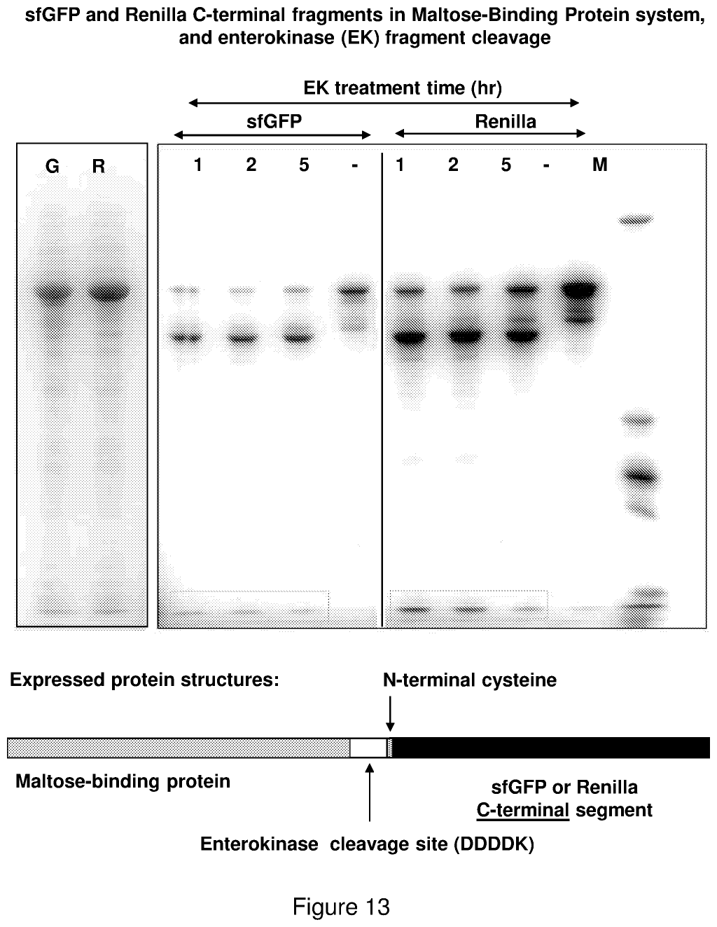

Coding sequences for each C-terminal segment (boundaries as described in Example 1) were provided with cysteine codons at the desired N-terminal positions and, in addition, equipped with an enterokinase recognition signal (codons for DDDDK; SEQ ID NO:44), such that after expression, the C-terminal fragments can be cleaved from the maltose binding carrier protein. Assembled sequences were cloned between Xmn I and Sbf I sites of pMALc5x (New England Biolabs), and the structure of candidate clones confirmed by sequencing. Verified clones were transformed into the strain NEB-express (New England Biolabs), and propagated in liquid culture (50 ml) under short-term growth conditions at 37° C. for 1.5 hours, before induction with 300 μM IPTG for a further 2 hours. Samples (200 μl “direct lysates”) were taken, pelleted in 1.5 ml tubes at 1000 g, washed once with 200 μl of 1×PBS, and resuspended in 50 μl of PBS. The remainder of the 50 ml growths were pelleted (10 minutes at 3000 rpm in a Sorvall benchtop centrifuge), and resuspended in 2.0 ml Eppendorf tubes in 1.5 ml of ice-cold maltose-binding protein system column buffer (MC-buffer; 20 mM Tris pH 7.4, 200 mM NaCl, 1 mM EDTA, and 1 mM DTT), and then sonicated and clarified to yield soluble supernatants, by means of the same conditions as used in Example 1. On SDS-PAGE gels, such supernatants showed strong bands of the expected molecular weights (see, FIG. 13, Lanes G and R, for sfGFP and Renilla preparations, respectively). The C-terminal fragments of these model reporter proteins have similar molecular weights (9.1 and 9.4 kD for sfGFP and Renilla, respectively). Thus, the observed MBP fusion protein bands for both fragments migrate at an expected size of about 51 kD (see, FIG. 13).

Polypeptides expressed as fusions with maltose-binding protein were affinity purified on amylose magnetic beads (A-MBs; New England Biolabs). Suitable samples of A-MBs (usually equivalent to 250 μl of the original slurries per 1 ml of supernatant) were washed twice with 1 ml of cold MC-buffer (using magnetic separation to pull down the A-MBs), and resuspended in the original volume. Sonicated supernatants from induced plasmid cultures were mixed with the A-MBs for 1 hour at 4° C., with frequent tube inversion to resuspend the beads. Following this, the supernatants were magnetically removed, and the beads washed four times with 0.5 ml of cold MC-buffer before resuspension in 150 μl of the same buffer per 250 μl of original beads. Bound proteins were then eluted with a final concentration of 10 mM maltose for 1 hour at 25° C.

Isolated protein fusions with MBP then require treatment with enterokinase in order to release free polypeptide fragments of interest from the MBP carrier. Both fusions were cleavable, producing the expected fragments (see, FIG. 13). Under the conditions of this Example, cleavage with a constant amount of enterokinase peaked by 1 hour and was not further enhanced by extended incubation times (see, FIG. 13).

### Example 4: Expression and Purification of N-terminal sfGFP and Renilla Fragments as Maltose-Binding Protein Fusions, and Fragment Cleavage with Enterokinase

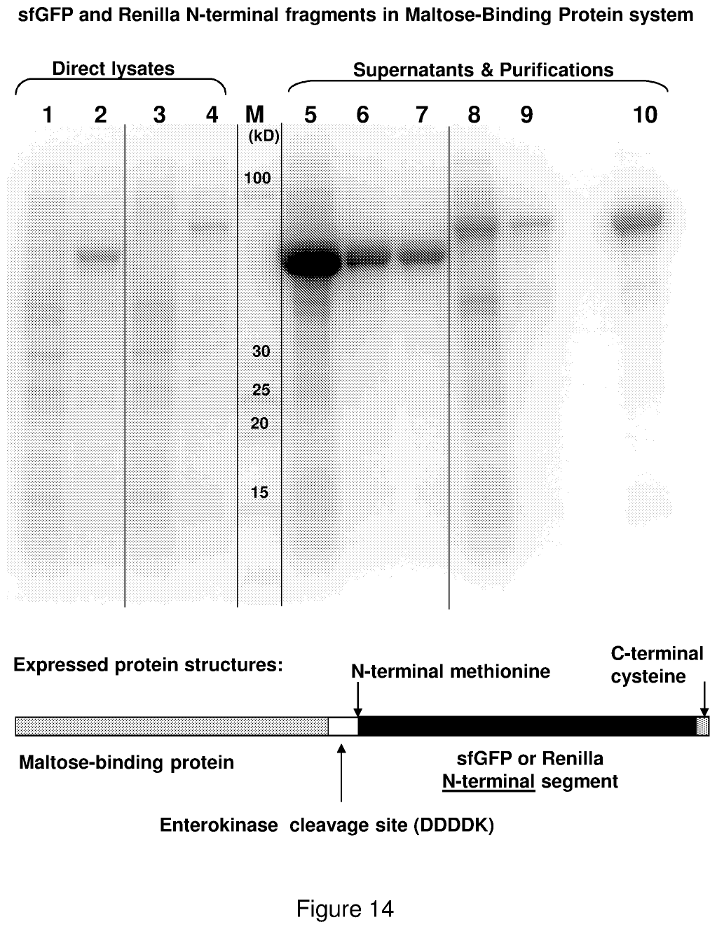

Since the Renilla luciferase N-terminal fragment was refractory to soluble fusion with an intein-chitin binding domain fusion (see, Example 1), the MBP system for expression of N-terminal fragments for SP-TAPER as C-terminal MBP fusions was used. The N-terminal coding sequence for Renilla, as used for the intein-based system (see, Example 1), was adapted for in-frame C-terminal expression as an MBP fusion via amplification of coding sequence with primers bearing the appropriate alterations, using a proof-reading DNA polymerase system (Phusion, Thermo). At the same time, similar manipulations were performed on corresponding sfGFP sequence for comparative purposes. Amplified segments were digested with Xmn I and Sbf I (present in the primers but not within the coding sequences) and inserted into pMALc5x in a similar manner as for Example 3. In this case, however, the cysteine codon was placed at the 3′ end of these coding sequences, such that cysteine residues would be expressed at the C* termini. As in the C-terminal fragment expression (see, Example 3), a cleavage site for enterokinase was also inserted between the end of MBP coding sequence and the beginning of coding sequence for the N-terminal sfGFP and Renilla fragments (schematically depicted in FIG. 14). The structures of candidate clones were confirmed by sequencing. Verified clones were transformed into the strain NEB-express (New England Biolabs), and propagated for IPTG induction in the same manner as for Example 3, as were direct lysate samples, sonication for initial supernatant generation, and binding and elution from amylose magnetic beads (A-MBs).

It was found that both sfGFP and Renilla N-terminal fragments could be expressed as soluble fusion proteins with MBP. Both were observed in direct lysate samples on SDS-PAGE gels only after IPTG induction (see, FIG. 14, Lanes 1 vs. 2; and Lanes 3 vs. 4). Moreover, both of the induced fusion bands were present in sonicated supernatants (see, FIG. 14, Lanes 5 and 8, for sfGFP and Renilla N-terminal fragments respectively). In turn, both could be bound eluted from A-MBs with maltose (see, FIG. 14, Lanes 6 and 9, for sfGFP and Renilla N-terminal fragments respectively).

Under the conditions used, the elution was more efficient for the sfGFP fragment. Samples of the A-MBs bearing fusion proteins bound via MBP were taken (after washing four times with MC-buffer as in Example 3) as homogeneous slurries. For comparison, samples of post-elution supernatants were also taken, where the volumes of the initial slurries and the maltose-eluted soluble material were the same. These pairs were denatured as usual in Laemmli buffer at 100° C. (as in Example 1), and 5 μl samples loaded onto an SDS-PAGE gel. Since the A-MB slurry sample represents total bound protein present, comparison of its band intensity with that of the volume-matched eluted sample provides an estimate of elution efficiency. Thus, the sfGFP N-terminal fusion showed excellent elution approaching completion, whereas the soluble yield of N-terminal Renilla fusion was reduced (see, FIG. 14, Lane 6 vs. Land 7; and Lane 9 vs. Lane 10, respectively). Nevertheless, purified soluble N-terminal Renilla fusion protein was producible in the MBP system, in strong distinction to the failure to observe equivalent soluble protein in the intein-based system of Example 1.

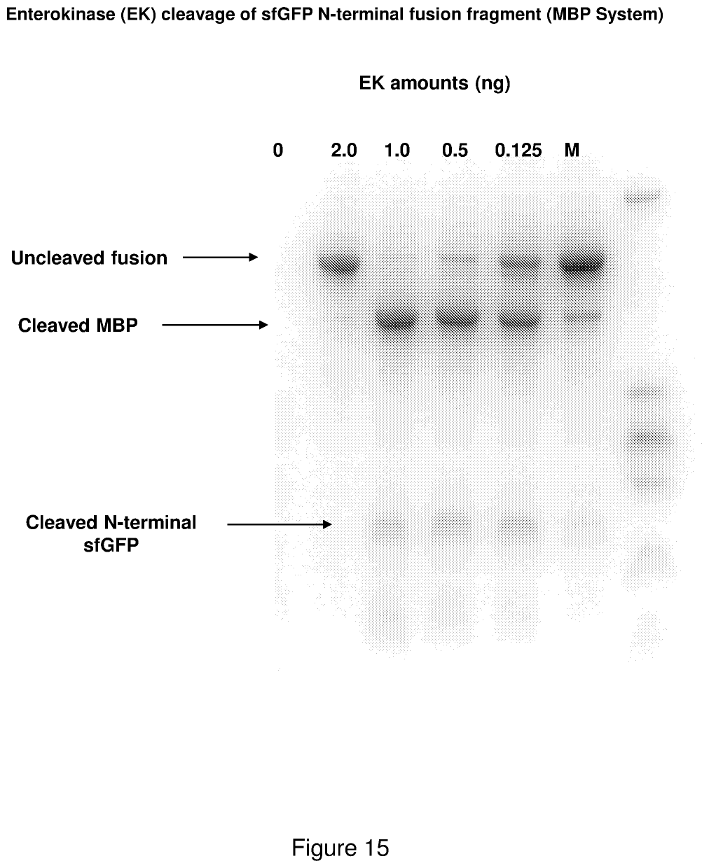

The N-terminal sfGFP fusion with MBP was further examined by treatment with enterokinase, in order to liberate the free N-terminal fragment. When varying amounts of enterokinase were used with a fixed amount of sfGFP fusion for 2.5 hours at 25° C., a dose-response was observed, with near-total cleavage with the greatest amount of protease (see, FIG. 15). At the same time, the released fragment (about 17 kD) could be detected on SDS-PAGE gels (see, FIG. 15).

### Example 5: Chemical Ligation of a Nucleic Acid Tag with a 5′ or 3′ Sulfhydryl Group and a Polypeptide with an N-Terminal or C-Terminal Cysteine

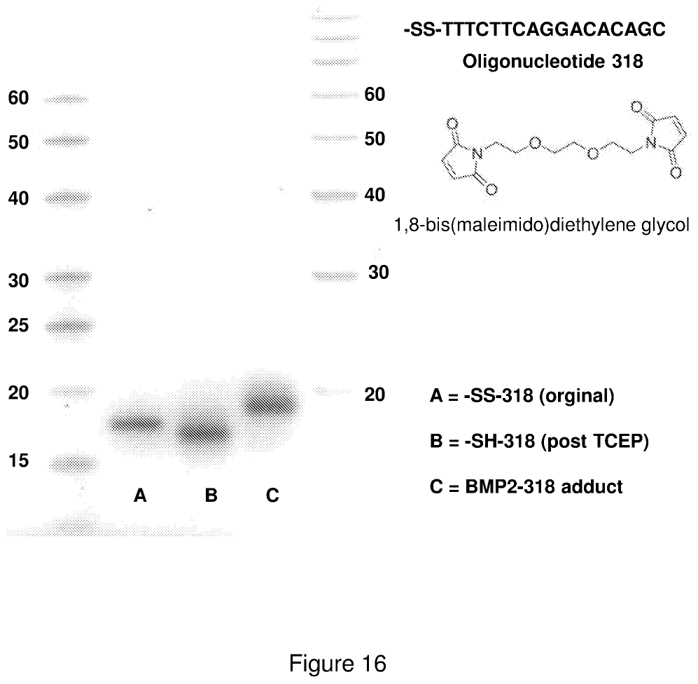

The conjugation process using bis-maleimide linkers is performed in two stages. Initially, oligonucleotides bearing a 5′ or 3′ terminal disulfide modification (see, FIG. 16; -SS-TITCTTCAGGACACAGC; SEQ ID NO:45) are treated with 100-fold molar excess of TCEP for at least 4 hours at 25° C., and then desalted into 10 mM Tris pH 7.4 to remove the TCEP and low-molecular weight products. The resulting —SH oligonucleotides are then treated with a molar excess (500-fold) of BMP2 (Sigma) in sodium phosphate buffer pH 7.1 for 4 hours at 25° C. The preparations are then desalted once more to remove excess BMP2. Samples of the modified oligonucleotides are run on 8 M urea gels to examine the success of the process, in comparison to the original -SS- oligonucleotides and the corresponding derived —SH oligonucleotides (see, FIG. 16).

The second stage uses the BMP2-derivatized oligonucleotide to cross-link to a polypeptide fragment of interest with a terminal cysteine residue. Suitable fragments are incubated in phosphate buffer (pH 7.1) with 100 mM sodium chloride, and a large molar excess of BMP2-derivatized oligonucleotide to drive the reaction, for 4 hours at 25° C. Excess oligonucleotides (bearing unreacted maleimide groups) are then removed by treatment with sulfhydryl magnetic beads (Bioclone). Polypeptide conjugates are then dialysed against PBSM and stored in 50% glycerol at −20° C.

### Example 6: Assembly and Functionality of Reporter Fragments on a Cell Surface by SP-TAPER

The process of cell-surface assembly and assay of reporter fragments by templated SP-TAPER can be divided into several stages, which include:

1) placing a nucleotide sequence for templating purposes on a cell surface in a specific manner;

2) choice of reporter cleavage points for SP-TAPER;

3) preparation of reporter cleavage-point polypeptides, and their conjugation with nucleic acid tags for SP-TAPER;

4) determining reassembly of reporter cleavage fragments by specifically-templated SP-TAPER in an in vitro system; and

5) demonstration of effectiveness of cell surface template for an SP-TAPER reporter system.

This Example describes each stage of this process. Stage 5 is not undertaken until the previous stages 1-4 have been successfully demonstrated.

### 1) Surface Template:

SP-TAPER uses a target nucleic acid molecule sequence as a template for assembling protein fragments for targeted assembly on a cell surface. An initial aspect is a means for localizing a template sequence on a target cell in a specific manner. Aptamers can be used for this purpose. In such circumstances, aptamers can be viewed as bifunctional entities consisting of both a recognition segment (for binding to a cell surface target) and a template sequence, either at a 5′ or 3′ terminus of a singlet aptamer, or at the 5′-3′ junction of a binary aptamer. An example of an aptamer against a surface marker in melanoma (the melanocortin-1 receptor (MC1R) a G-protein coupled receptor transducing signals from alpha-melanocyte stimulating hormone) has also been described.

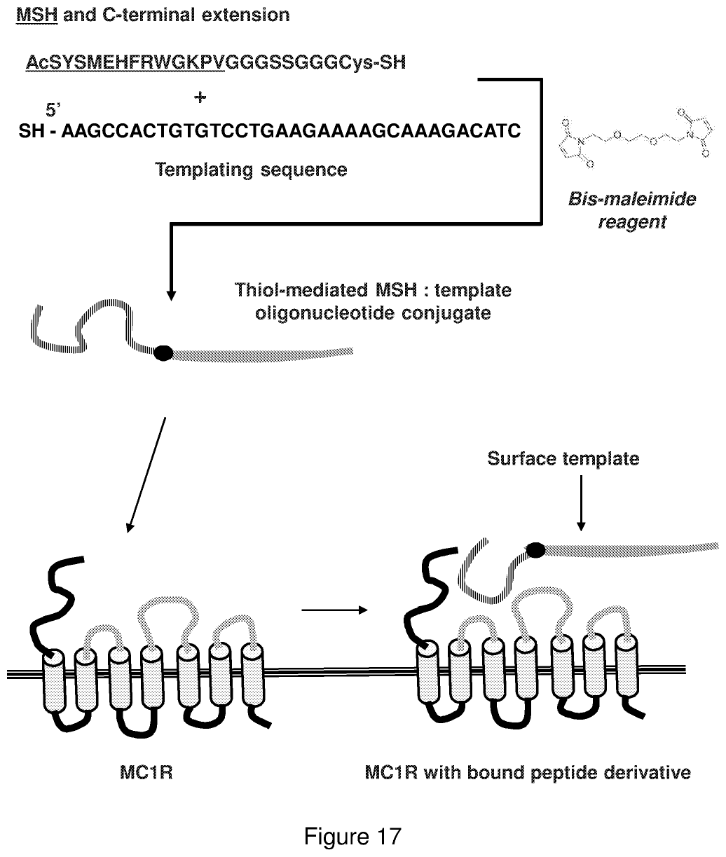

An alternative approach to generating a surface template exists when a ligand for a surface target is known. In this Example, the ligand is alpha-melanocyte stimulating hormone (MSH), with a C-terminal extension comprised of a serine-glycine linker, and a C-terminal cysteine residue (see, FIG. 17; AcSYSMEHFRWGKPVGGGSSGGGC-SH; SEQ ID NO:21). This terminal cysteine enables formation of an oligonucleotide conjugate, where the oligonucleotide bears a 5′ (or 3′) —SH group, via a bis-maleimide cross-linking reagent (see, FIG. 17). In this Example, the displayed template sequence corresponds to a segment of human papillomavirus 16 E6/E7 sequence (see, FIG. 17; AAGCCACTGTGTCCTGAAGAAAAGCA AAGACATC; SEQ ID NO: 20).

In variants of this Example, the MSH ligand is substituted to produce enhanced binding properties. In one example, NDP-MSH is produced whereby the extended version of NDP-MSH for templating has the amino acid sequence of AcSYSXEHFRWGKPVGGGSSGGGC (SEQ ID NO:22), where the wild-type Met-4 and Phe-7 residues (both bolded) are replaced by norleucine (Nle) and D-phenylalanine (D-Phe) respectively. Other variants of the MSH ligand include, CSGGGSSGGGSYSMEHFRWGKPV-NH₂ (SEQ ID NO:23), and CSGGGSSGGGSYSXEHFR WGKPV-NH₂ (SEQ ID NO:24), wherein X is norleucine and the F residue is D-phenylalanine.

The conjugation process using bis-maleimide linkers is performed according to the two-stage protocol of Example 5, using a 100:1 molar ratio of BMP2-modified template oligonucleotide (see, FIG. 17) to synthetic peptide, such that derivatization of the peptide is driven towards completion. Following this, excess unreacted BMP2-oligonucleotide is removed by reaction with sulfhydryl-modified long-arm magnetic beads (Bioclone) to transfer any remaining maleimide oligonucleotide to the solid phase. The soluble phase is subsequently partitioned from the beads by magnetic separation.

To display the prepared surface template, cells (2.105) are treated with 1 nmol of peptide ligand-template conjugates for 1 hour on ice, and washed twice with 1×PBS with 1 mM MgCl₂ (PBSM). Positive control cells are known to express surface MC1R; negative controls are MC1R-; both types of cells are also treated in the same manner but with the exclusion of the peptide ligand-template conjugates. Both the binding of the receptor ligand and the presence of accessible surface template are assayed simultaneously by means of a fluorescent bilabeled probe that is complementary to the appended template tag: 5′-6Fam-GATGTCTTGCTTTCT TCAGGACACAGTGGCTT-6Fam (SEQ ID NO:46).

The bilabeled probe (500 pmol) is added to the cells (0.5 ml; 2.105) bearing peptide ligand-templates, and matched control cells as defined above. After 30 minutes at 25° C. incubation, cells are re-washed once with PBSM, and then subjected to flow analysis with channel settings as for fluorescein. Successful ligand binding and template accessibility is defined by significant fluorescent peaks for MC1R+ cells while absent from MC1R-cells, where both were pre-treated with the peptide ligand-template conjugate, and also absent from all cells where the peptide ligand-template conjugate was omitted.

### 2) Choice of Reporter Cleavage Points for SP-TAPER:

The placements of cleavage points for the reporters sfGFP and Renilla luciferase are as described in Example 1.

3) Preparation of Reporter Cleavage-Point Polypeptides, and their Conjugation with Nucleic Acid Tags for SP-TAPER:

Examples 1-4 describe methods for the preparation of cleavage-point polypeptides for the reporters sfGFP and Renilla luciferase. Either the intein-based system or MBP system are applicable to the N-terminal sfGFP fragment, while the MBP system is successful with the N-terminal fragment of Renilla luciferase, and C-terminal fragments for both reporters. Methods for preparation of polypeptide-nucleic acid tag conjugates via sulfhydryl groups and bis-maleimide chemical linkers are as described in Example 5.

A locked TAPER first bottle haplomer oligonucleotide (bearing a 5′—SH group) with a loop region complementary to the predetermined template sequence (see, FIG. 17) is separately conjugated with the C-terminal cysteines of N-terminal sfGFP and Renilla luciferase fragments (as defined in Example 1). The corresponding second haplomer (bearing a 3′—SH group) is separately conjugated with the N-terminal cysteines of C-terminal sfGFP and Renilla luciferase fragments (as also defined in Example 1). Both types of conjugates are schematically depicted in FIG. 9.

### 4) Testing for Reassembly of Reporter Cleavage Fragments by Specifically-Templated SP-TAPER in an In Vitro System:

Correctly reassembled reporter polypeptide fragments will by definition be proficient for their inherent “reportable” functions. In this Example, a linear DNA template (corresponding to the free oligonucleotide version of the template in FIG. 17) is used with the locked TAPER oligonucleotide reporter conjugates described above. Since template titration effects are avoided by the use of locked TAPER systems, an excess of template may be used with variable amounts of the conjugated first haplomer bottle and second haplomers.

The following conjugates are prepared as described above:

| | Oligonucleotide | |
|---|---|---|
| Protein fragment | (attachment detail) | Code name |
| sfGFP N-terminal | Locked TAPER first | sfG-N-H1 |
| | haplomer bottle (5′ end to | |
| | C*-terminus of N-terminal | |
| | fragment) | |
| sfGFP C-terminal | Locked TAPER second | sfG-C-H2 |
| | haplomer (3′ end to N* | |
| | terminus of C-terminal | |
| | fragment) | |
| Renilla luciferase N- | Locked TAPER first | R-N-H1 |
| terminal | haplomer bottle (5′ end to | |
| | C*-terminus of N-terminal | |
| | fragment) | |
| Renilla luciferase C- | Locked TAPER second | R-C-H2 |
| terminal | haplomer (3′ end to N* | |
| | terminus of C-terminal | |
| | fragment) | |

The sfGFP signal is fluorescence at the same emission maximum as for fluorescein, and is monitored by means of a spectrophotometer with fluorescent reading facility (Tecan). The enzymatic activity of Renilla luciferase is assessed by means of commercial kits for this enzyme (Promega), using coelenterazine substrate, and purified Renilla luciferase (RayBiotech) as a positive control. Luminescence is quantified by means of a standard luminometer (Berthold).

In a dose-response experimental design, equimolar amounts of (sfG-N-H1+sfG-C-H2) and (R—N-H1+R—C-H2) are mixed in dilution series ranging from 10.0 to 0.1 pmol each, in 2-fold dilution steps, or as the available quantities of SP-haplomers permit, before mixing with a constant amount of DNA target template, in a two-fold excess over the highest quantity of conjugates used. After a 16 hour incubation at 25° C., reporter signals are assayed as appropriate for both sfGFP and Renilla luciferase.

A comparable time-course experiment may also be performed, where a constant amount of polypeptide conjugates ([sfG-N-H1+sfG-C-H2] and [R—N-H1+R—C-H2]) are mixed with a two-fold excess of template, with assayable samples taken at a series of time points: 15, 30, 45, 60 minutes; and 1, 2, 4, 6, 8, and 16 hours.

Specificity of the template-mediated polypeptide assembly may be demonstrated by the use of blocking oligonucleotides that correspond to the same sequences as used oligonucleotide-polypeptide conjugates (as depicted in FIG. 9) but without the appended polypeptide tags. A molar excess of either of these oligonucleotides effectively inhibits the assembly reaction, whereas the assembly process is unaffected by excess oligonucleotides of the same length but with scrambled sequence.

### 5) Demonstration of Effectiveness of Cell Surface Template for an SP-TAPER Reporter System:

In this Example, surface template is generated on target cells expressing MC1R, in the manner described above (see, FIG. 17). Cells used include the melanoma line 453A, and the lymphoma line K562, both of which are known to possess surface MC1R as detected by primary antibody and FITC-labeled secondary (Santa Cruz Biotechnology). Following confirmation that the template is displayed and accessible (by means of a bilabeled fluorescent probe, as above), the template-displaying cells are treated with an excess of pairs of polypeptide conjugates ([sfG-N-H1+sfG-C-H2] and [R—N-H1+R—C-H2]) and incubated for 2 hours at 25° C. Generation of reporter signals through surface-templated co-folding of polypeptide fragments for sfGFP is effected by flow analysis, in comparison with cells treated only with anti-MC1R primary and fluorescent secondary antibodies. For cell-surface determination of Renilla luciferase, whole-cell samples are assayed directly for luminescence as described above, with intact Renilla enzyme as the positive assay control.

### Example 7: Assembly and Functionality of Toxin Fragments on a Cell Surface by SP-TAPER

The process of cell-surface assembly of a small toxic mediator and its ensuing functional activity in uptake and cell killing can be divided into several stages, which include:

1) placing a nucleotide sequence for templating purposes on a cell surface in a specific manner;

2) choice of polypeptide toxin, and its cleavage points for SP-TAPER;

3) preparation of toxin cleavage-point polypeptides, and their conjugation with nucleic acid tags for SP-TAPER;

4) examination of reassembly of toxin cleavage fragments by specifically-templated SP-TAPER in an in vitro system;

5) demonstration of effectiveness of cell surface template by an SP-TAPER reporter system; and

6) demonstration of cell killing by uptake of surface-assembled toxin.

This Example describes each stage of this process. Stages 5 and 6 are not undertaken until the previous stages 1-4 have been successfully demonstrated.

### 1) Surface Template:

The methods for deriving cell-surface nucleic acids for the purpose of acting as templates for SP-TAPER is as described in Example 6.

### 2) Polypeptide Toxin and Cleavage Points:

Although a number of small ribotoxins are attractive from the viewpoint of their potential applications towards SP-TAPER, Hirsutellin A (HstA) is the leading contender as the smallest known to date, and where potential cleavage points can be identified (as above; see, FIG. 10). The initial cleavage point selected is a diglycine at positions 89 and 90 of the mature polypeptide (see, FIG. 10).

A useful control to include in the SP-TAPER work with HstA is a mutant lacking a catalytically crucial residue within the C-terminal fragment, Histidine-113 (see, FIG. 10; converting the normal codon to that encoding a glycine residue (H113G), by a point mutational change).

3) Preparation and nucleic acid conjugation of toxin fragment polypeptides:

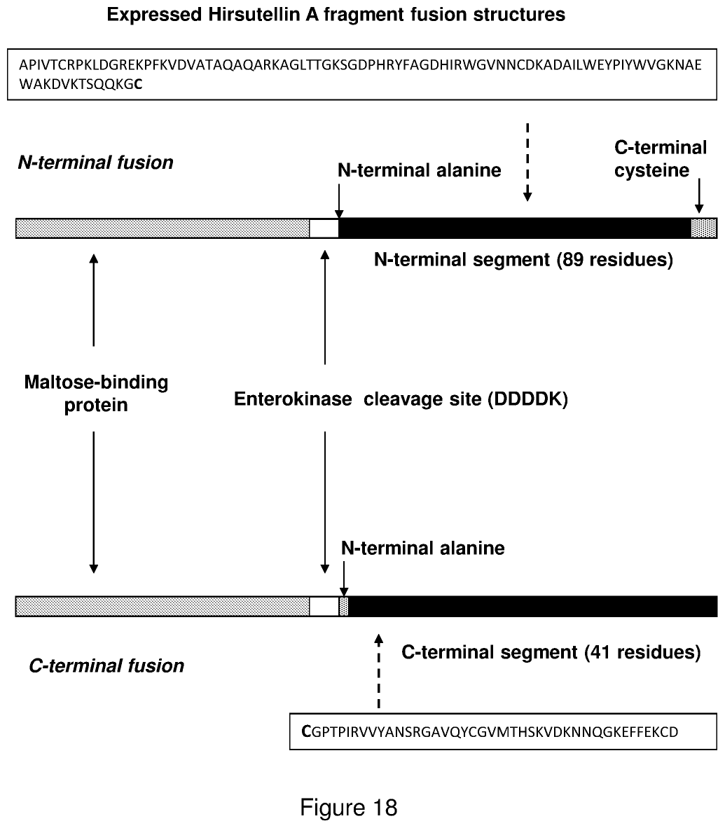

Both the N-terminal (APIVTCRKLDGREKPFKVDVATAQAQARKAGLTTGKSGDP HRYFAGDHIRWGVNNCDKADAILWEYPIYWVGKNAEWAKDVKTSQQKGC; SEQ ID NO:47) and C-terminal HstA (CGPTPIRVVYANSRGAVQYCGVMTHSKVDKNNQGKEFFE KCD; SEQ ID NO:26) fragments at the diglycine site are expressed as fusions in the MBP system, with inserted C-terminal and N-terminal cysteine residues, respectively, by means of synthetic coding sequences (see, FIG. 18). Fusion proteins are expressed, bound to amylose magnetic beads, and eluted with maltose as described in Examples 3 and 4. The polypeptide HstA fragments are liberated with enterokinase (Examples 3 and 4), followed by enterokinase removal with a commercial affinity product (Thermofisher).

A locked TAPER bottle oligonucleotide (first haplomer bottle; see, FIG. 8) is prepared with a 5′ sulfhydryl group and an anti-target loop portion complementary to the target nucleic acid molecule template sequence of interest. In this example, the template is surface-displayed on MC1R+ cells, as shown in FIG. 16. The 5′ sulfhydryl is generated from a disulfide precursor by treatment with TCEP followed by desalting. This locked TAPER bottle oligonucleotide is conjugated with the N-terminal HstA fragment bearing a C-terminal cysteine (see, FIG. 17) by means of a bifunctional maleimide reagent in the same manner as for Example 5.

The corresponding second haplomer oligonucleotide for locked TAPER (see, FIG. 8) is likewise prepared with a free 3′-sulfhydryl group, and then conjugated with the N-terminal HstA fragment bearing an N-terminal cysteine (see, FIG. 17) also by means of a bifunctional maleimide reagent as for Example 5.

Each conjugate is purified by excision from a native acrylamide gel, followed by electroelution. Although both HstA fragments contain internal cysteines, undesired conjugates involving these residues are resolvable from single N- or C-terminal conjugates in appropriate gel systems. While the single-terminal conjugates approximate to a linear backbone structure spanning the polypeptide chain to the appended nucleic acid phosphodiester sequence, one or more internal conjugates possess a branched structure that results in altered electrophoretic mobilities.

### 4) Testing for Reassembly of Toxin Cleavage Fragments by Specifically-Templated SP-TAPER:

The two HstA-locked TAPER conjugates (50 pmol each, prepared as above) are incubated with and without a two-fold excess of free templating sequence (as in FIG. 17, but unconjugated) in 1×PBSM for 6 hours at 25° C. To assay for the effects of correctly assembled HstA, a mammalian in vitro translation system is appropriate. A coupled in vitro transcription/translation system based on rabbit reticulocyte lysate preparations (Promega TNT® Quick Coupled Transcription Translation System) is conveniently used to generate a sensitive read-out in the form of luciferase, plasmids for which (and testing reagents) are included in the commercial kit. Ribotoxins, including HstA, interfere with ribosomal protein synthesis, allowing assayable protein production to serve as a gauge for the level of ribotoxin activity.

The system is established for control luciferase production according to the manufacturer's instructions, and seeded with increasing amounts of the test-assembled HstA preparations before incubating for 90 minutes at 37° C. Controls include HstA polypeptide conjugates without template, and the addition of unlabeled blocking oligonucleotides with the same sequences as the conjugates (as described above for the reporter assembly systems).

Positive controls are represented by a commercial sample of another ribotoxin (ricin A chain, Sigma), and HstA itself, expressed in E. coli. The latter is produced from full-length synthetic coding sequence inserted into the pMALc5x vector in an analogous manner as to other expressed polypeptides in this application, where the full-length HstA polypeptide is cleaved from the MBP carrier via enterokinase. Following purification of the MBP-HstA fusion on amylose-magnetic beads, elution with maltose, and enterokinase cleavage, the protease is removed with a commercial affinity resin (EMD-Millipore), and the preparation used directly for testing with the luciferase in vitro transcription/translation system.

A successful read-out in this system is achieved with a dose-dependent diminution of the luciferase luminescent signal by the assembled HstA fragments, in parallel with the ribotoxin positive controls. The assembly process is demonstrated to be template-dependent, and specifically blockable with unlabeled competitor oligonucleotides.

In parallel with the suppression of luciferase reporter activity, HstA assembly can also be addressed directly with the same in vitro assay components, by means of assessing ribosomal 28S RNA cleavage at the sarcin-ricin loop. Following the in vitro transcription-translation process, with and without assembled HstA preparations, samples of the whole reaction mixes are phenol extracted, precipitated, and reconstituted in TE buffer under RNase-free conditions. Samples are run on 2% agarose and visualized with ethidium bromide (Kao et al., Meth. Enzymol., 2001, 341, 324-335). Generation of the characteristic 400-base ribotoxin α-fragment is diagnostic of successful HstA assembly. Additionally, a synthetic 35-mer corresponding to the sarcin-ricin loop in 28S RNA (GGUAAUCCUGCUCAGUACGAGAGGAACCGCAGGUU; SEQ ID NO:48; Endo et al., J. Biol. Chem., 1998, 263, 7917-7920) can be used to directly assay for specific ribotoxin cleavage in vitro. This is performed by incubating the oligoribonucleotide with increasing amounts of the test-assembled HstA (and whole-HstA control preparations) before incubating for 90 minutes at 37° C. Products and successful cleavage are assessed on 15% 8 M urea denaturing acrylamide gels.

In parallel with the SP-TAPER analyses with wild-type HstA conjuagtes, a conjugate bearing the H113G mutation (C-terminal SP-haplomer) is used to demonstrate the specificity of the effect on suppression of eukaryotic ribosomal protein synthesis.

### 5) Demonstration of Effectiveness of Cell Surface Template by an SP-TAPER Reporter System:

The accessibility of the cell surface template for the purposes of toxin assembly is initially confirmed by means of reporter assembly by SP-TAPER, as described in Example 6.

### 6) Demonstration of Cell Killing by Uptake of Surface-Assembled Toxin:

The established cell surface template system (as described in Example 6) is used. HstA polypeptide conjugates and control sfGFP and Renilla reporter conjugates are incubated in a dilution series with cells previously manipulated to display surface template. Parallel experiments with in vitro templates for HstA assembly act as positive controls for the assembly process itself. Additionally, the parallel generation of cell surface reporter signal indicate that the template system is functional as planned.

The desired activity of surface-assembled and functional HstA is dependent on its uptake into the target cells from the surface site. This occurs actively through inherent cell-penetrating functionality of the mature HstA protein, or passively in any case via endocytosis.

The cytotoxic effect is monitored and quantitated by direct microscopy with vital stains, and via flow analysis with commercial Annexin V systems for apoptotic cells.

### Example 8: Functional SP-TAPER with sfGFP Split Protein System

To demonstrate the efficacy of SP-TAPER, the following components were used:

### A. Protein Fragments:

The following protein fragment components of a specific version of sfGFP (Overkamp et al., Applied Environ. Microbiol., 2013, 79, 6481-6490) were expressed in E. coli NiCo21(DE3):

N-terminal:
(SEQ ID NO: 53)
MSKGEELFTGVVPILVELDGDVNGHKFSVRGEGEGDATNGKLTLKFI
CTTGKLPVPWPTLVTTLTYGVQCFSRYPDHMKRHDFFKSAMPEGYVQ
ERTISFKDDGTYKTRAEVKFEGDTLVNRIELKGIDFKEDGNILGHKL
EYNFNSHNVYITADKQGGSGHHHHHH;
C-terminal:
(SEQ ID NO: 54)
MHHHHHHGGSGKNGIKANFKIRHNVEDGSVQLADHYQQNTPIGDGPV
LLPDNHYLSTQSVLSKDPNEKRDHMVLLEFVTAAGITHGMDELYK;

Appended hexhistidine tags are shown in bold; short serine-glycine linkers between the hexahistidine segments and sfGFP sequences are underlined. Purifications from sonicated cell extracts were effected on Immobilized Metal Affinity Chromatography magnetic beads (IMAC, Dynabeads, Thermofisher Corp), and proteins eluted with 300 mM imidazole.

### B. Oligonucleotides for Creating Protein—Nucleic Acid Conjugates:

The following oligonucleotides were used to implement Locked-TAPER for application to this instance of SP-TAPER.

(i) TrisAm-HPV-B1 (Locked TAPER haplomeric loop
‘bottle’ oligo)
(SEQ ID NO: 28)
[Tris-tandem amino]-ACTCGAGACGTCTCCTTGTCTTTGCTT
TTCTTCAGGACACAGTGGCGAGACGTCTCGAGT;
and
(ii) HPV-B2-TrisAm (Second Locked TAPER
haplomeric oligo)
(SEQ ID NO: 58)
TTTGACGTCTCGAGT-[Tris-tandem amino].

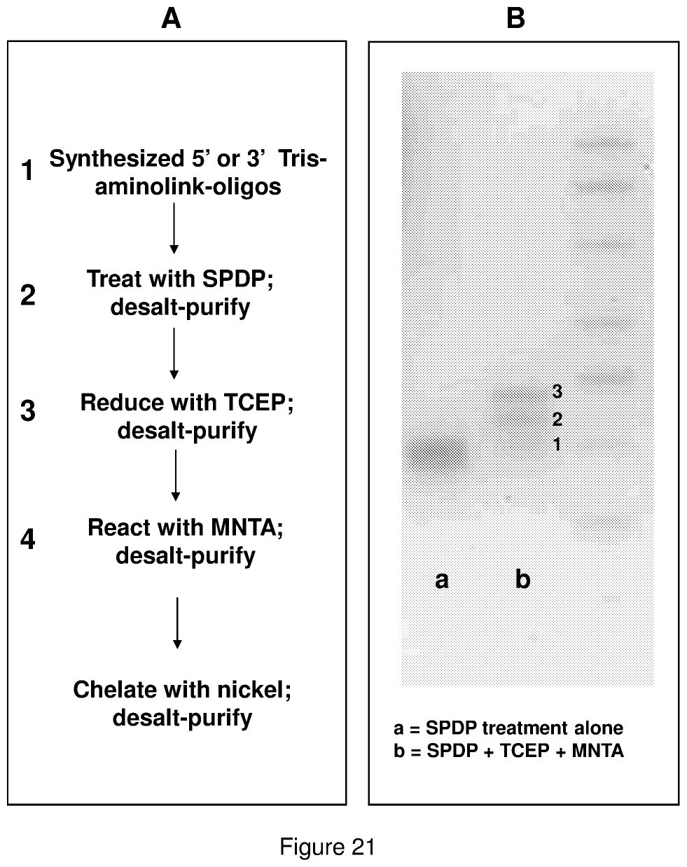

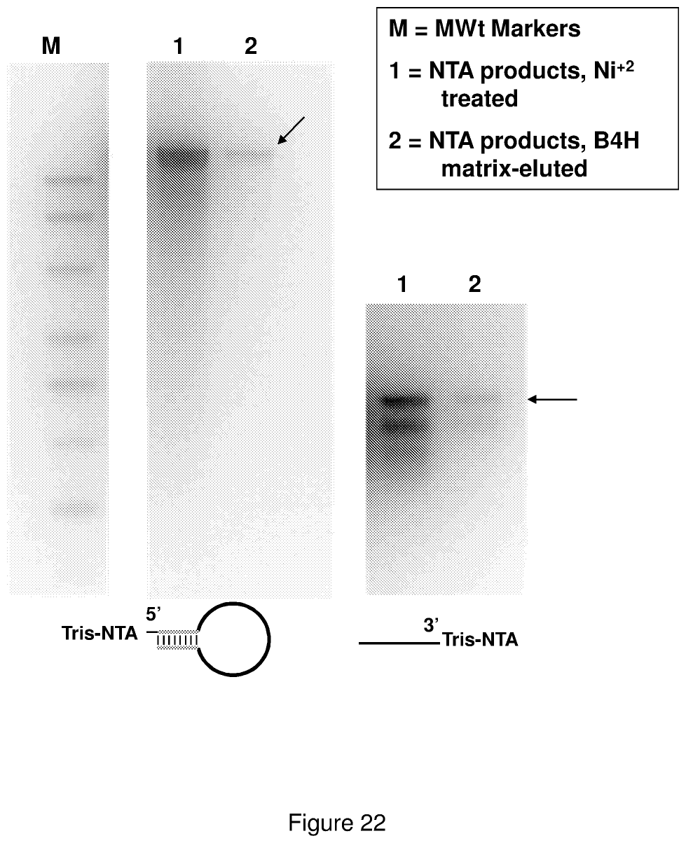

These oligonucleotides TN-HPV-B1 and HPV-B2-T were synthesized with tris-tandem amino groups at their 5′ or 3′ ends, respectively, to enable their tandem derivitization with maleimido-C3-nitrilotriacetic acid (MNTA; Dojindo Molecular Technologies), to increase the binding affinity in the presence of Ni⁺² for hexahistidine tags (see, Goodman et al., Chembiochem, 2009, 10, 1551-1557). This MNTA conjugation proceeds as described above (see, FIG. 19), except for the iteration of the terminal amino groups into a triple set. The conjugation process uses an initial step where the tris-terminal amines are initially converted into dithiols with the bifunctional reagent N-succinimidyl 3-(2-pyridyldithio) propionate (SPDP), followed by reduction with TCEP and finally conjugation via the maleimido-moiety of MNTA (see, FIG. 21, panel A). In practice, although the conjugation reaction itself readily proceeds, it is not possible to achieve fully Tris-substituted derivatization of oligonucleotides, as shown in FIG. 21, panel B with the non-limiting example of the small component of a Locked-TAPER system with mono-, di-, and tri-substituted NTA forms evident on a denaturing acrylamide gel. Thus, enrichment of the trisubstituted NTA form is desirable. This was effected by means of the biotinylated tetrahistidine strategy as described for purification of single-MNTA conjugates (see, FIG. 20). Here reaction products for mixed tandem MNTA products are charged with Ni⁺² ions to allow chelation, incubated with solid-phase biotinylated tetrahistidine, and eluted with imidazole. This process allowed a selective enrichment of the tris-tandem NTA form of Locked TAPER oligonucleotides by virtue of its higher affinity towards tetrahistidine (see, FIG. 22).

When split-protein fragments are conjugated with specific oligonucleotides designed to hybridize in mutual proximity on a common template, they are termed SP-haplomers. When combined with the Locked-TAPER strategy, they are accordingly termed (in abbreviation) as Lk-SP-haplomers.

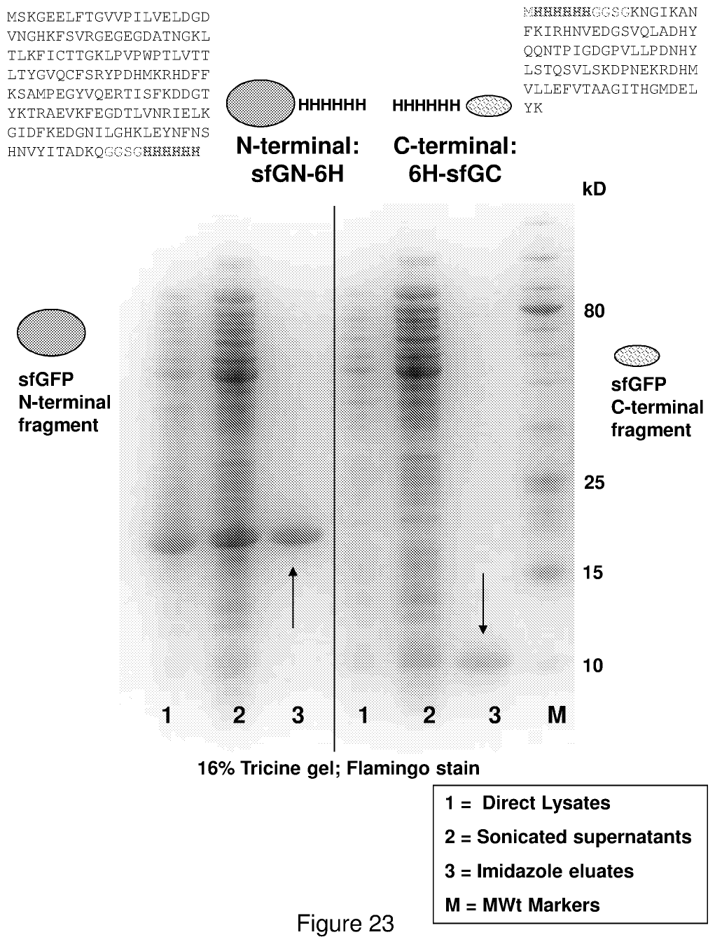

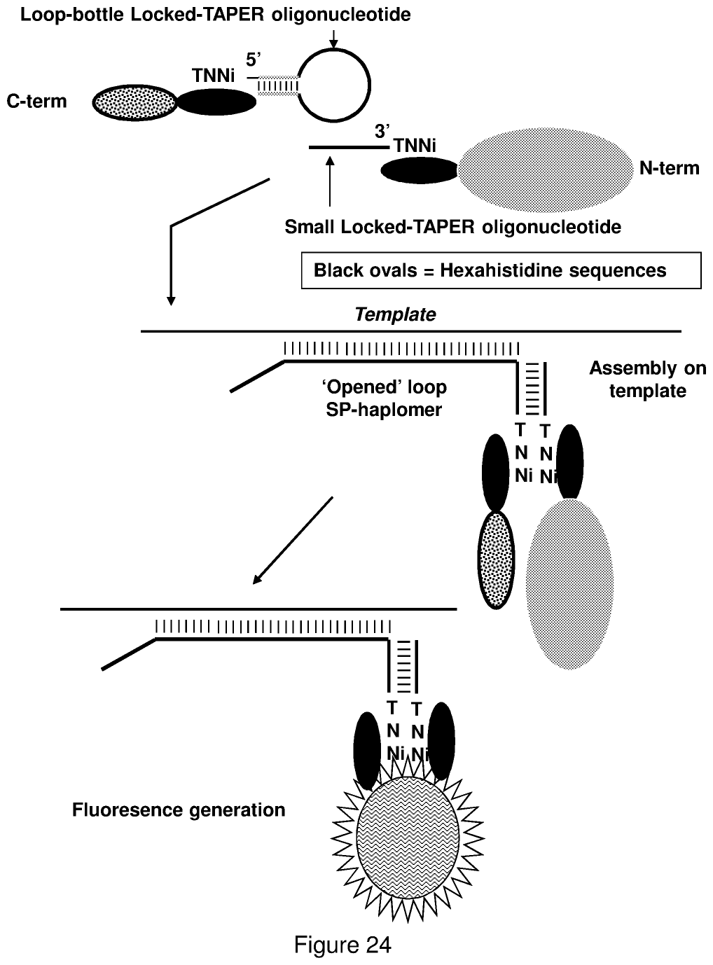

Fragments of sfGFP (as above) bearing hexahistidine tags were expressed in E. coli strain NiCo21(DE3) and purified according to standard protocols, with a final elution via imidazole (see, FIG. 23). These preparations were then derivatized with Tris-tandem-NTA Locked TAPER oligonucleotides (see, FIG. 22), where the proteins were in molar excess.

In this non-limiting example, 300 pmol of the larger sfGFP fragment (see, FIG. 23) was treated with 160 pmol of the small Locked-TAPER oligonucleotide HPV-B2 (as above) derivatized and enriched as above to form HPV-B2-3′-Tris-NTA. In this non-limiting example, 300 pmol of the smaller sfGFP fragment (see, FIG. 23) was treated with 160 pmol of the larger loop-bottle Locked-TAPER oligonucleotide (as above) HPV-B1, derivatized and enriched as above to form 5′ Tris-NTA-HPV-B1. By using an excess of protein, complexing of the oligonucleotides was driven towards completion, thus minimizing the amounts of free oligonucleotides which can interfere with subsequent templated SP-TAPER.

For demonstration of SP-TAPER, the above Lk-SP-haplomers (20 pmol each) were incubated either separately or together in a 50 μl volume in 50 mM phosphate buffer pH 7.0/100 mM NaCl, with a 10-fold excess (200 pmol) of template oligonucleotide directly in a 96-well black-sided flat-bottomed plate (Corning): TAACTGTCAAAAGCCACTGTGTCCTGA AGAAAAGCAAAGACATCTGGACAAAAAGC (SEQ ID NO:59); or a 10-fold excess (200 pmol) of a corresponding scrambled oligonucleotide: TAACTGTCAAAAGCCACAAGCGGAA TAATGACTTCCCAGGGATAGATCAAAAAGC (SEQ ID NO:49).

By using Locked-TAPER, the template concentration may be used in large excess over the Lk-SP-haplomers, since the template titration effect observed with conventional haplomers is circumvented.

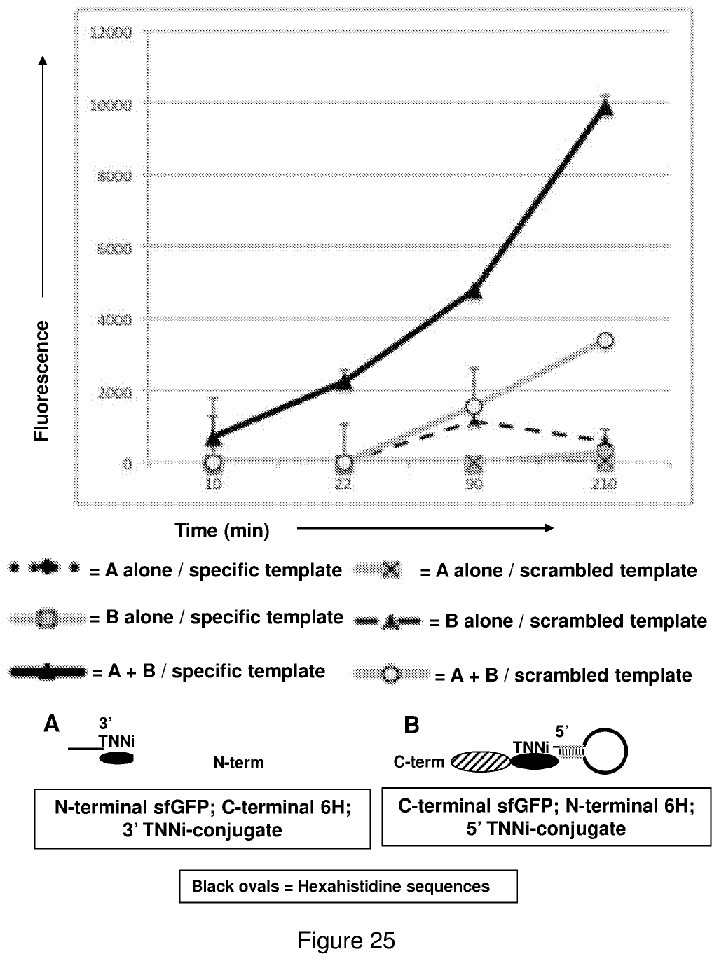

At suitable time-points, the plate was read for fluorescence (settings as for fluorescein) in a Tecan spectrofluorimeter. Results are shown in FIG. 25. The fluorescence response, indicative of sfGFP activity, was greatly accelerated in the presence of specific over scrambled template. No significant response was observed with either Lk-SP-haplomer alone, irrespective of template.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A bottle haplomer comprising a polynucleotide, wherein the polynucleotide comprises:
a) a first 3′ stem portion comprising from about 10 to about 20 nucleotide bases;
b) an anti-target loop portion comprising from about 16 to about 40 nucleotide bases linked to the first 3′ stem portion, wherein the anti-target loop portion is substantially complementary to a target nucleic acid molecule; and
c) a second 5′ stem portion comprising from about 10 to about 20 nucleotide bases linked to the anti-target loop portion, wherein the first 3′ stem portion is substantially complementary to the second 5′ stem portion;
wherein:
the 5′ terminus of the polynucleotide comprises a —SH moiety, and
the Tₘ of the anti-target loop portion:target nucleic acid molecule is greater than the Tₘ of the first stem portion:second stem portion; or
wherein the polynucleotide comprises:
a) a first 3′ stem portion comprising from about 10 to about 20 nucleotide bases;
b) an anti-target loop portion comprising from about 16 to about 40 nucleotide bases linked to the first 3′ stem portion, wherein the anti-target loop portion is substantially complementary to a target nucleic acid molecule; and
c) a second 5′ stem portion comprising from about 10 to about 20 nucleotide bases linked to the anti-target loop portion, wherein the first 3′ stem portion is substantially complementary to the second 5′ stem portion:
wherein:
the Tₘ of the anti-target loop portion:target nucleic acid molecule is greater than the Tₘ of the first stem portion:second stem portion; and
the 5′ terminus or 3′ terminus of the polynucleotide is linked to the C-terminus of an N-terminal protein fragment or the N-terminus of a C-terminal protein fragment, wherein the terminus of the protein fragment lined to the polynucleotide comprises a cysteine or selenocysteine.

2. (canceled)

3. The bottle haplomer according to claim 1 wherein:
the Tₘ of the first stem portion:second stem portion subtracted from the Tₘ of the anti-target loop portion:target nucleic acid molecule is from about 10° C. to about 40° C.;
the Tₘ of the first stem portion:second stem portion is from about 40° C. to about 50° C.;
the Tₘ of the anti-target loop portion:target nucleic acid molecule is from about 60° C. to about 80° C.; and/or
the Tₘ of the first stem portion:second stem portion subtracted from the Tₘ of the anti-target loop portion:target nucleic acid molecule is from about 10° C. to about 20° C.

4. The bottle haplomer according to claim 1 wherein:
the first stem portion comprises from about 12 to about 18 nucleotide bases;
the anti-target loop portion comprises from about 18 to about 35 nucleotide bases; and/or
the second stem portion comprises from about 12 to about 18 nucleotide bases.

5. The bottle haplomer according to claim 1 further comprising a linker between any one or more of the first stem portion and the anti-target loop portion, or between the anti-target loop portion and the second stem portion.

6. A haplomer comprising:
a) a polynucleotide; and
b) an N-terminal protein fragment or a C-terminal protein fragment, wherein the 3′ or 5′ terminus of the polynucleotide is linked to the N-terminus of the C-terminal protein fragment or the C-terminus of the N-terminal protein fragment;
wherein:
the N-terminal fragment comprises the amino acid sequence of APIVTCRKLDGREKP FKVDVATAQAQARKAGLTTGKSGDPHRYFAGDHIRWGVNNCDKADAILWEYPIYWVG KNAEWAKDVKTSQQKG (SEQ ID NO:1), and the C-terminal fragment comprises the amino acid sequence of GPTPIRVVYANSRGAVQYCGVMTHSKVDKNNQGKEFFEKCD (SEQ ID NO:2);
the N-terminal fragment comprises the amino acid sequence of APIVTCRPKLDG (SEQ ID NO:3), and the C-terminal fragment comprises the amino acid sequence of REKPFKVDVAT AQAQARKAGLTTGKSGDPHRYFAGDHIRWGVNNCDKADAILWEYPIYWVGKNAEWA KDVKTSQQKGGPTPIRVVYANSRGAVQYCGVMTHSKVDKNNQGKEFFEKCD (SEQ ID NO:4);
the N-terminal fragment comprises the amino acid sequence of APIVTCRPKLDGREKP FKVDVATAQAQARKAGLTTGK (SEQ ID NO:5), and the C-terminal fragment comprises the amino acid sequence of SGDPHRYFAGDHIRWGVNNCDKADAILWEYPIYWVGKNAEWA KDVKTSQQKGGPTPIRVVYANSRGAVQYCGVMTHSKVDKNNQGKEFFEKCD (SEQ ID NO:6);
the N-terminal fragment comprises the amino acid sequence of APIVTCRPKLDGR EKPFKVDVATAQAQARKAGLTTGKSGDPHRYFAGDHIRWGVNNCDKAD (SEQ ID NO:7), and the C-terminal fragment comprises the amino acid sequence of AILWEYPIYWVGK NAEWAKDVKTSQQKGGPTPIRVVYANSRGAVQYCGVMTHSKVDKNNQGKEFFEKCD (SEQ ID NO:8);
the N-terminal fragment comprises the amino acid sequence of APIVTCRPKLDGR EKPFKVDVATAQAQARKAGLTTGKSGDPHRYFAGDHIRWGVNNCDKADAILWEYPIY WVG (SEQ ID NO:9), and the C-terminal fragment comprises the amino acid sequence of KNAEWAKDVKTSQQKGGPTPIRVVYANSRGAVQYCGVMTHSKVDKNNQGKEFFEKCD (SEQ ID NO:10);
the N-terminal fragment comprises the amino acid sequence of APIVTCRPKLDGR EKPFKVDVATAQAQARKAGLTTGKSGDPHRYFAGDHIRWGVNNCDKADAILWEYPIY WVGKNAEWAKD (SEQ ID NO:11), and the C-terminal fragment comprises the amino acid sequence of VKTSQQKGGPTPIRVVYANSRGAVQYCGVMTHSKVDKNNQGKEFFEKCD (SEQ ID NO:12);
the N-terminal fragment comprises the amino acid sequence of APIVTCRPKLDGR EKPFKVDVATAQAQARKAGLTTGKSGDPHRYFAGDHIRWGVNNCDKADAILWEYPIY WVGKNAEWAKDVKTSQ (SEQ ID NO:13), and the C-terminal fragment comprises the amino acid sequence of QKGGPTPIRVVYANSRGAVQYCGVMTHSKVDKNNQGKEFFEKCD (SEQ ID NO:14);
the N-terminal fragment comprises the amino acid sequence of APIVTCRPKLDGR EKPFKVDVATAQAQARKAGLTTGKSGDPHRYFAGDHIRWGVNNCDKADAILWEYPIY WVGKNAEWAKDVKTSQQKGGPTPIRVVYANSRG (SEQ ID NO:15), and the C-terminal fragment comprises the amino acid sequence of AVQYCGVMTHSKVDKNNQGKEFFEKCD (SEQ ID NO:16);
the N-terminal fragment comprises the amino acid sequence of APIVTCRPKLDGR EKPFKVDVATAQAQARKAGLTTGKSGDPHRYFAGDHIRWGVNNCDKADAILWEYPIY WVGKNAEWAKDVKTSQQKGGPTPIRVVYANSRGAVQYCGVMTHSKVDKN (SEQ ID NO:17), and the C-terminal fragment comprises the amino acid sequence of NQGKEFFEKCD (SEQ ID NO:18); or
the N-terminal fragment comprises the amino acid sequence of APIVTCRPKLDGREK PFKVDVATAQAQARKAGLT; (SEQ ID NO:40), and the C-terminal fragment comprises the amino acid sequence of TGKSGDPHRYFAGDHIRWGVNNCDKADAILWEYPIYWVGKNA EWAKDVKTSQQKGGPTPIRVVYANSRGAVQYCGVMTHSKVDKNNQGKEFFEKCD (SEQ ID NO:41).

7-9. (canceled)

10. A fusion protein comprising:
an N-terminal protein fragment, a fusion partner protein, and a purification domain, wherein the C-terminus of the N-terminal protein fragment is coupled to the N-terminus of the fusion partner protein, and the C-terminus of the fusion partner protein is coupled to the N-terminus of the purification domain; or
an N-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the N-terminal protein fragment, wherein the N-terminal protein fragment comprises an N-terminal methionine and a C-terminal cysteine; or
a C-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the C-terminal protein fragment, wherein the C-terminal protein fragment comprises an N-terminal cysteine.

11. The fusion protein according to claim 10 comprising:
an N-terminal protein fragment, intein, and a chitin-binding domain, wherein the C-terminus of the N-terminal protein fragment is coupled to the N-terminus of intein, and the C-terminus of intein is coupled to the N-terminus of the chitin-binding domain; or
an N-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the N-terminal protein fragment, wherein the N-terminal protein fragment comprises an N-terminal methionine and a C-terminal cysteine; or
a C-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the C-terminal protein fragment, wherein the C-terminal protein fragment comprises an N-terminal cysteine.

12. The fusion protein according to claim 11 comprising an N-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the N-terminal protein fragment, wherein the N-terminal protein fragment comprises the amino acid sequence
(SEQ ID NO: 25)
APIVTCRPKLDGREKPFKVDVATAQAQARKAGLTTGKSGDPHRYFAG
DHIRWGVNNCDKADAILWEYPIYWVGKNAEWAKDVKTSQQKGC.

13. The fusion protein according to claim 10 comprising a C-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the C-terminal protein fragment, wherein the C-terminal protein fragment comprises the amino acid sequence
(SEQ ID NO: 26)
CGPTPIRVVYANSRGAVQYCGVMTHSKVDKNNQGKEFFEKCD.

14. (canceled)

15. A composition or kit comprising:
a) a first haplomer, wherein the first haplomer comprises a polynucleotide linked to the C-terminus of an N-terminal protein fragment; and
b) a second haplomer, wherein the second haplomer comprises a polynucleotide linked to the N-terminus of a C-terminal protein fragment;
wherein:
the polynucleotide of one of the first or second haplomers is linked at its 5′ terminus to the protein fragment, and the other of the first and second haplomers is linked at its 3′ terminus to the protein fragment;
the N-terminal protein fragment and the C-terminal protein fragment are derived from a single protein; and
wherein:
the polynucleotide of the first haplomer is complementary to the polynucleotide of the second haplomer; or
the polynucleotide of the first haplomer is complementary to a target nucleic acid molecule, and the polynucleotide of the second haplomer is substantially complementary to the target nucleic acid molecule at a site in spatial proximity to the polynucleotide of the first haplomer; or
the polynucleotide of the first haplomer is substantially complementary to a portion of a target nucleic acid molecule 5′ adjacent to a stem-loop structure, and the polynucleotide of the second haplomer is substantially complementary to a portion of the target nucleic acid molecule 3′ adjacent to the stem-loop structure; or
the polynucleotide of the first haplomer is substantially complementary to a 5′ portion of a loop of a stem-loop structure of a target nucleic acid molecule, and the polynucleotide of the second haplomer is substantially complementary to a 3′ portion of the loop of the stem-loop structure of the target nucleic acid molecule; or
a composition or kit comprising:
a) a bottle haplomer comprising a polynucleotide comprising:
i) a first 3′ stem portion comprising from about 10 to about 20 nucleotide bases;
ii) an anti-target loop portion comprising from about 16 to about 40 nucleotide bases linked to the first 3′ stem portion, wherein the anti-target loop portion is substantially complementary to a target nucleic acid molecule; and
iii) a second 5′ stem portion comprising from about 10 to about 20 nucleotide bases linked to the anti-target loop portion, wherein the first 3′ stem portion is substantially complementary to the second 5′ stem portion:
wherein:
the 5′ terminus of the polynucleotide comprises a —SH moiety; and
the Tₘ of the anti-target loop portion:target nucleic acid molecule is greater than the Tₘ of the first stem portion:second stem portion:
b) an N-terminal protein fragment, wherein the C-terminus of the N-terminal protein fragment comprises a cysteine-SH moiety; and
c) a bis-maleimide reagent; or
a composition or kit comprising:
a) a bottle haplomer comprising a polynucleotide comprising:
i) a first 3′ stem portion comprising from about 10 to about 20 nucleotide bases;
ii) an anti-target loop portion comprising from about 16 to about 40 nucleotide bases linked to the first 3′ stem portion, wherein the anti-target loop portion is substantially complementary to a target nucleic acid molecule; and
iii) a second 5′ stem portion comprising from about 10 to about 20 nucleotide bases linked to the anti-target loop portion, wherein the first 3′ stem portion is substantially complementary to the second 5′ stem portion:
wherein the 5′ terminus of the polynucleotide is linked to the C-terminus of an N-terminal protein fragment, wherein the C-terminus comprises a cysteine; and
b) a second haplomer comprising a polynucleotide and a C-terminal protein fragment, wherein the 3′ terminus of the polynucleotide is linked to the N-terminus of the C-terminal protein fragment, wherein the N-terminus comprises a cysteine:
wherein:
the polynucleotide of the second haplomer is substantially complementary to the second 5′ stem portion of the polynucleotide of the bottle haplomer;
the Tₘ of the anti-target loop portion:target nucleic acid molecule is greater than the Tₘ of the first stem portion:second stem portion; and
the N-terminal protein fragment and the C-terminal protein fragment are derived from a single protein.

16-17. (canceled)

18. The kit or composition according to claim 15 wherein the polynucleotide and protein fragment each comprise a bio-orthogonal reactive molecule.

19. A method for the directed assembly of a protein in a cell comprising:
a) contacting a cell with a first haplomer comprising a polynucleotide linked to the C-terminus of an N-terminal protein fragment; and
b) contacting the cell with a second haplomer comprising a polynucleotide linked to the N-terminus of a C-terminal protein fragment;
wherein:
the polynucleotide of one of the first or second haplomers is linked at its 5′ terminus to the protein fragment, and the other of the first and second haplomers is linked at its 3′ terminus to the protein fragment;
the N-terminal protein fragment and the C-terminal protein fragment are derived from a single protein; and
wherein:
the polynucleotide of the first haplomer is substantially complementary to the polynucleotide of the second haplomer; or
the polynucleotide of the first haplomer is substantially complementary to a target nucleic acid molecule, and the polynucleotide of the second haplomer is substantially complementary to the target nucleic acid molecule at a site in spatial proximity to the polynucleotide of the first haplomer; or
the polynucleotide of the first haplomer is substantially complementary to a portion of a target nucleic acid molecule 5′ adjacent to a stem-loop structure, and the polynucleotide of the second haplomer is substantially complementary to a portion of the target nucleic acid molecule 3′ adjacent to the stem-loop structure; or
the polynucleotide of the first haplomer is substantially complementary to a 5′ portion of a loop of a stem-loop structure of a target nucleic acid molecule, and the polynucleotide of the second haplomer is substantially complementary to a 3′ portion of the loop of the stem-loop structure of the target nucleic acid molecule;
thereby resulting in the assembly of the protein from the N-terminal protein fragment and the C-terminal protein fragment; or
a method for the directed assembly of a protein comprising:
a) contacting a target nucleic acid molecule with a bottle haplomer comprising:
i) a first 3′ stem portion comprising from about 10 to about 20 nucleotide bases;
ii) an anti-target loop portion comprising from about 16 to about 40 nucleotide bases linked to the first 3′ stem portion, wherein the anti-target loop portion is substantially complementary to a target nucleic acid molecule; and
iii) a second 5′ stem portion comprising from about 10 to about 20 nucleotide bases linked to the anti-target loop portion, wherein the first 3′ stem portion is substantially complementary to the second 5′ stem portion;
wherein the 5′ terminus of the polynucleotide is linked to the C-terminus of an N-terminal protein fragment, wherein the C-terminus comprises a cysteine; and
b) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer:
wherein:
the N-terminal protein fragment and the C-terminal protein fragment are derived from a single protein;
the Tₘ of the anti-target loop portion:target nucleic acid molecule is greater than the Tₘ of the first stem portion:second stem portion; and
the Tₘ of the duplex formed by the second haplomer and the second stem portion of the bottle haplomer subtracted from the Tₘ of the first stem portion:second stem portion is from about 0° C. to about 20° C.;
thereby resulting in the assembly of the protein from the N-terminal protein fragment and the C-terminal protein fragment; or
a method for the directed assembly of a protein comprising:
a) contacting a cell with a surface target compound comprising:
i) a template polynucleotide; and
ii) a peptide:
wherein:
the 5′ terminus of the polynucleotide is coupled to the N-terminus or C-terminus of the peptide, or the 3′ terminus of the polynucleotide is coupled to the N-terminus or C-terminus of the peptide; and
the peptide is a ligand for a cell-surface molecule;
b) contacting the cell with a first haplomer comprising a polynucleotide linked to the C-terminus of an N-terminal protein fragment; and
c) contacting the cell with a second haplomer comprising a polynucleotide linked to the N-terminus of a C-terminal protein fragment:
wherein:
the polynucleotide of one of the first or second haplomers is linked at its 5′ terminus to the protein fragment, and the other of the first and second haplomers is linked at its 3′ terminus to the protein fragment;
the N-terminal protein fragment and the C-terminal protein fragment are derived from a single protein; and
the polynucleotide of the first haplomer is substantially complementary to the template polynucleotide of the surface target compound, and the polynucleotide of the second haplomer is substantially complementary to the template polynucleotide of the surface target compound at a site in spatial proximity to the polynucleotide of the first haplomer;
thereby resulting in the assembly of the protein from the N-terminal protein fragment and the C-terminal protein fragment; or
a method for the directed assembly of a protein comprising:
a) contacting a cell with a surface target compound comprising:
i) a template polynucleotide; an:
ii) a peptide:
wherein:
the 5′ terminus of the polynucleotide is coupled to the N-terminus or C-terminus of the peptide, or the 3′ terminus of the polynucleotide is coupled to the N-terminus or C-terminus of the peptide; and
the peptide is a ligand for a cell-surface molecule;
b) contacting a target nucleic acid molecule with a bottle haplomer comprising:
i) a first 3′ stem portion comprising from about 10 to about 20 nucleotide bases;
ii) an anti-target loop portion comprising from about 16 to about 40 nucleotide bases linked to the first 3′ stem portion, wherein the anti-target loop portion is substantially complementary to the template polynucleotide of the surface target compound; and
iii) a second 5′ stem portion comprising from about 10 to about 20 nucleotide bases linked to the anti-target loop portion, wherein the first 3′ stem portion is substantially complementary to the second 5′ stem portion;
wherein the 5′ terminus of the polynucleotide is linked to the C-terminus of an N-terminal protein fragment, wherein the C-terminus comprises a cysteine; and
c) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer:
wherein:
the N-terminal protein fragment and the C-terminal protein fragment are derived from a single protein;
the Tₘ of the anti-tar et loop portion:target nucleic acid molecule is greater than the Tₘ of the first stem portion:second stem portion; and
the Tₘ of the duplex formed by the second haplomer and the second stem portion of the bottle haplomer subtracted from the Tₘ of the first stem portion:second stem portion is from about 0° C. to about 20° C.;
thereby resulting in the assembly of the protein from the N-terminal protein fragment and the C-terminal protein fragment.

20-23. (canceled)
